(19) United States
(12) Patent Application Publication
(10) Pub. No.: US 2024/0401048 A1
(43) Pub. Date: Dec. 5, 2024

(54) MICRORNA-541-3P AS A THERAPEUTIC AGENT AS WELL AS ZNF101 AND CASZ1 AS THERAPEUTIC TARGETS TO LOWER PLASMA LDL-C, INCREASE PLASMA HDL-C, AND REDUCE ATHEROSCLEROSIS

(71) Applicants: New York University, New York, NY (US); The United States Government As Represented By The Department of Veterans Affairs, Washington, DC (US)

(72) Inventors: Abulaish Ansari, Zone 52 (QA); Pradeep Kumar Yadav, Varanasi (IN); M. Mahmood Hussain, Woodbury, NY (US)

(21) Appl. No.: 18/735,087

(22) Filed: Jun. 5, 2024

Related U.S. Application Data
(60) Provisional application No. 63/506,311, filed on Jun. 5, 2023.

(51) Int. Cl. C12N 15/113; A61P 9/10; C12N 9/22; C12N 15/11

(52) U.S. Cl. CPC C12N 15/113; A61P 9/10; C12N 9/22; C12N 15/111; C12N 2310/141; C12N 2310/20; C12N 2320/32

(57) ABSTRACT

Provided are compositions and methods for lowering plasma LDL-C, increasing plasma HDL-C, and reducing atherosclerosis. The methods include administering to an individual in need miRNA-541-3P or a modified version thereof, or an agent other than the miRNA-541-3P or the modified version thereof that inhibits the function or expression of ZNF101 or CASZ1, or a combination thereof. Performing a method also decreases expression of apoB increases expression of apoA1 is increased.

### CROSS-REFERENCE TO RELATED APPLICATIONS

This application claims the benefit of U.S. provisional patent application No. 63/506,311, filed Jun. 5, 2023, the entire disclosure of which is incorporated herein by reference.

### STATEMENT REGARDING FEDERALLY SPONSORED RESEARCH OR DEVELOPMENT

This invention was made with government support under RO1 HL166214, RO1 HL160470, RO1 HL158054 and RO1 HL137202 awarded by the National Institutes of Health and BX004113 awarded by The United States Government as represented by the Department of Veterans Affairs. The government has certain rights in the invention.

### SEQUENCE LISTING

The instant application contains a Sequence Listing, which is submitted in .xml format and is hereby incorporated by reference in its entirety. Said .xml file is named “058636_00698_ST26.xml”, was created on Jun. 4, 2024, and is 112,114 bytes in size.

### RELATED INFORMATION

High plasma cholesterol levels are risk factors for atherosclerotic cardiovascular disease (ASCVD) (1, 2). Cholesterol is carried in the blood by apoB-containing low-density lipoproteins (apoB-Lps) and apoA1-containing high-density lipoproteins (HDL). ApoB-Lps are synthesized and secreted by the liver and the intestine to deliver endogenous and dietary lipids to peripheral tissues. Excess accumulation of modified apoB-Lps in the plasma, and their uptake by macrophages, contributes to ASCVD (3, 4). HDL extracts and transports free cholesterol from macrophages and other peripheral cells to the liver for excretion from the body (5, 6). Hence, low HDL is also associated with ASCVD. Therefore, understanding physiological and molecular mechanisms controlling plasma low-density lipoproteins (LDL) and HDL levels will aid in developing new therapeutics against ASCVD. To this end, LDL-lowering therapies are widely used to reduce heart disease. Yet, a significant portion of the population does not respond and/or is intolerant to these drugs (7, 8). Therefore, drugs have been developed to lower LDL by inhibiting hepatic production of apoB-Lps (9). However, these approaches are associated with hepatosteatosis (10). Furthermore, increasing plasma HDL therapeutically has not been effective in lowering ASCVD (6, 11). Notably, agents that concurrently decrease LDL and increase HDL have not been identified. Hence, there is a need to develop new LDL lowering therapies and to increase functional HDL levels. The present disclosure is related to this need.

### BRIEF SUMMARY

High apoB-containing low-density lipoproteins (LDL) and low apoA1-containing high-density lipoproteins (HDL) are associated with atherosclerosis. The present disclosure provides agents that simultaneously and reciprocally control both LDL and HDL levels. The disclosure provides a screen of a microRNA (miR) library using human hepatoma Huh-7 cells and demonstrates that miR-541-3p both significantly decreases apoB and increases apoA1 expression by inducing mRNA degradation of two different transcription factors, Znf101 and Casz1. The disclosure demonstrates that Znf101 enhances apoB expression while Caz1 represses apoA1 expression. The hepatic knockdown of Casz1 increased plasma apoA1, HDL and cholesterol efflux capacity. The hepatic knockdown of Zfp961, an ortholog of Znf101, reduced lipogenesis, production of TG rich lipoproteins and atherosclerosis without causing hepatic lipid accumulation. The disclosure therefore provides compositions and methods that increase the amount of miR-541-3p or a derivative thereof in cells, and for inhibiting the function and/or expression of hepatic Znf101/Zfp961 and/or Casz1 to alter plasma lipoproteins and reduce atherosclerosis without causing liver steatosis. As such, in an example, the disclosure provides a method comprising administering to an individual in need thereof a composition comprising: i) miRNA-541-3P or a modified version thereof; or ii) an agent other than the miRNA-541-3P or the modified version thereof that inhibits the function or expression of ZNF101; or iii) an agent other than the miRNA-541-3P or the modified version thereof that inhibits the function or expression of CASZ1; iv) or a combination comprising at least two of i), ii) and iii).

In an example, a method of the disclosure comprising administering miRNA-541-3P or a modified version thereof, i.e., a derivative of miRNA-541-3P. In an example, a modified version of miRNA-541-3P comprises a modified ribosugar modification, a modified ribonucleotide, a modified inter-nucleoside linkage, or a combination thereof.

In examples, a method of the disclosure comprises administering an RNAi agent that inhibits expression of the ZNF101 protein, or inhibits expression of CASZ1 protein, or a combination of such RNAi agents. In examples, the method of the disclosure comprises administering an agent, such as a CRISPR system that modifies a genomic ZNF101 coding region such that expression of ZNF101 protein, or CASZ1, or a combination thereof, is reduced relative to expression of the described proteins by an unmodified cell, i.e., a cell wherein a gene encoding the described protein has not been modified.

In examples, the miRNA-541-3P or the modified version thereof or the agent is administered to liver cells within the individual.

In examples, performing a method of the disclosure is done with an individual who is in need of treatment or prophylaxis of atherosclerosis, or is in need of a reduction of plasma low-density lipoprotein (LDL), or is in need of an increase of plasma high-density lipoprotein (HDL), or a combination thereof. In examples, a reduction of the plasma LDL and an increase of the HDL occur. In examples, performing a method of the disclosure results in a condition within cells wherein expression of apoB is decreased and expression of apoA1 is increased, relative to expression of apoB and apoA1 by the same cell types that have not received a described polynucleotide or other described agent.

### BRIEF DESCRIPTION OF FIGURES

The patent or application file contains at least one drawing executed in color. Copies of this patent or patent application publication with color drawing(s) will be provided by the Office upon request and payment of the necessary fee.

FIGS. 1A-1C. miR-541-3p reciprocally regulates apoB and aopA1 secretion in human liver cells. (A-B). Huh-7 cells in 6-well plates were reverse transfected in triplicate with different amounts of miR-541-3p mimics or antimiR-541-3p. Control cells were transfected with a control miR (20 nM) and were used as 100% control. After 38 h, media was changed and collected after overnight incubation. ApoB (A) and apoA1 (B) levels were quantified in media and in cell lysates in triplicate by ELISA and normalized to cellular protein levels. Data are representative of 3 experiments. *, P<0.05; **, P<0.01; ***, P<0.001; **** P<0.0001, two-way ANOVA, non-parametric. (C) Human primary hepatocytes were seeded in 24-well plates. After one day, they were transfected in triplicate with miR-541-3p mimics or antimiR-541-3p (10 nM, n=3). After 16 h, media was replaced with fresh media. After 24 h, media was used to quantify apoB and aloA1 by ELISA in triplicates. *, P<0.05; **, P<0.01; unpaired t-test.

FIGS. 2A-2I. Regulation of apoB by miR-541-3p. (A-B) Huh-7 cells were transfected in triplicate with different amounts of miR-541-3p mimics or antimiR-541-3p, and APOB and ZNF101 mRNA levels were quantified in triplicate. (C) Cells were transfected in triplicate with different concentrations of specific siRNA against ZNF101 (siZnf101). After 48 h, mRNA levels of ZNF101, APOA1 and APOB were quantified (left). Media was used to quantify apoB and apoA1 protein levels (right). (D) Huh-7 cells were transfected in triplicate with siZnf101 (21 nM) or miR-541-3p mimics (10 nM), alone or in combination. Changes in ZNF101, APOA1, and APOB mRNA were quantified in triplicate in cell lysates (left), and protein from conditioned media (right). (E) Cells were forward transfected (n=3) with plasmids (5 μg) expressing the dual reporter Gaussia luciferase/secreted alkaline phosphatase under control of the Znf101 3′-UTR or a control plasmid without Znf101 3′-UTR. Next day, these cells were reverse transfected in triplicate with miR-541-3p mimics or antimiR-541-3p. After 48 h, media was measured for luciferase and alkaline phosphatase activities in triplicates. (F) Cells transfected (n=3) with miR-541-3p mimics (left) or antimiR-541-3p (right) were used for Ago2 immunoprecipitation and measurements of miR-541-3p and Znf101 mRNA. (G) Cells were transfected in triplicate with 20 nM miR-541-3p mimics or a control miR in triplicate. After 18 h, cells were washed and treated with actinomycin D (10 μg/mL). At indicated times, Znf101 and apoB mRNA levels were quantified in triplicates. (H) Cells were transfected in triplicate with plasmids expressing luciferase under the control of the apoB or cytomegalovirus promoter. Next day, cells were equally distributed in wells and transfected in triplicate with different amounts of miR-541-3p mimics or antimiR-541-3p. After 48 h, luciferase activity was measured in triplicates in conditioned media. (1) Cells were transfected in triplicate with plasmids expressing luciferase under control of the wild type or mutated apoB promoter. Next day, cells were equally distributed and transfected with different amounts of siZnf101 in triplicates. After 48 h, conditioned media was used to measure luciferase activity in triplicate. *P<0.05; **, P<0.01, ***P<0.001; ****P<0.0001. One-way ANOVA non-parametric test.

FIGS. 3A-3I. Regulation of apoA1 by miR-541-3p. (A-B) Huh-7 cells were forward transfected in triplicate with miR-541-3p mimics or antimiR-541-3p. After 48 h, mRNA levels of APOA1 and CASZ1 were quantified in triplicate. (C) SiCasz1 transfected cells (n=3) were collected to measure mRNA (left) and media to quantify apoB and apoA1 protein levels (right) in triplicates. (D) Cells were transfected in triplicate with 10 nM of siCtrl or siCasz1 or 21 nM miR-541-3p mimics, individually or in combination. Cells were used to measure different mRNAs (left) and media to measure apoB and apoA1 protein (right) in triplicate. (E) Plasmids for the expression of luciferase with or without the 3′-UTR of Casz1 were transfected (n=3). After 24 h, cells were reversed transfected in triplicate with different amounts of miR-541-3p mimics or antimiR-541-3p. After 24 h, luciferase activity was measured in triplicate in media. (F) Cells were transfected (n=3) with increasing amounts of miR-541-3p mimics or antimiR-541-3p. After 48 h, Ago2 immunoprecipitates were used to quantify miR-541-3p and CASZ1 mRNA levels in triplicates and expressed as a percent of control miR transfected cells. (G) Transfected cells were treated with actinomycin D (10 μg/mL) in triplicate and collected at different intervals to measure CASZ1 and APOA1 mRNA levels in triplicates. (H) Cells were transfected (n=3) with apoA1 promoter luciferase constructs. Next day, they were reverse transfected (n=3) with increasing concentrations of miR-541-3p mimics or antimiR-541-3p. After 48 h, luciferase activity was measured in triplicates. (1) Cells were transfected in triplicate with plasmids expressing luciferase under the control of the wild type or mutated apoA1 promoter. Next day, cells were equally distributed and transfected (n=3) with different amounts of siCasz1. After 48 h, luciferase activity was measured in triplicate in conditioned media. *P<0.05; **, P<0.01, ***P<0.001; ****P<0.0001. One-way ANOVA non-parametric test.

FIGS. 4A-4I. Hepatic knockdowns of Casz1 and Zfp961 alter plasma lipoproteins. Mice (C57Bl6J, female, 2.5-month-old) were transduced with AAV8 (2.5×10¹¹ gc/mouse) expressing shControl (shCtrl, n=4), shCasz1 (n=4), shZfp961 (n=4), or (sh(C+Z), n=5), and fed a Western diet. (A-D) After 6 weeks, livers were collected to measure mRNA levels in triplicates. (E-F) Plasma was collected from fasting mice to measure triglyceride and cholesterol (E) in technical duplicates, and subjected to precipitation using polyethylene glycol to measure cholesterol in HDL and non-HDL fractions (F) in triplicate. (G) Plasma (200 μL) obtained at the end of the study was subjected to FPLC, and triglyceride and cholesterol were measured in different fractions. *P<0.05; **, P<0.01, ***P<0.001; ****P<0.0001. One-way ANOVA non-parametric test. (H) Mice were gavaged with olive oil (100 μL) after overnight fast. Plasma was collected at different times to measure triglycerides in duplicates. Increases in plasma triglyceride were plotted as % of initial values before gavage. (1) Mice were fasted overnight prior to blood collection. Mice were injected intraperitoneally with Poloxamer P407 (1.5 mg/g). Blood was collected to measure triglyceride (left) in triplicate. Data between 2 and 4 h were used to calculate production rates (right).

FIGS. 5A-5F. Hepatic knockdown of Casz1 and Zfp961 in mutant PCSK9 transduced mice reduces atherosclerosis. All mice (C57Bl6J, male, 2.5-month-old) received AAV8 (5×10¹¹ gc) for the expression of mutant mouse gain-of-function Pcsk9 (mPcsk9). They were divided into four groups and received AAV8 for the expression of either shCtrl (5×10¹¹, n=4), shCasz1 (shCasz1 2.5×10¹¹+2.5×10¹¹ shCtrl, n=4) shZnf101 (shZnf101 2.5×10¹¹+shCtrl, 2.5×10¹¹, n=4) or shCasz1+shZnf101 (shCasz1, 2.5×10¹¹+shZnf101, 2.5×10¹¹, n=5) at the same time. Mice were fed an atherogenic Western diet. (A-B) Plasma was collected from overnight fasted mice to measure triglyceride, cholesterol, HDL-C, and non-HDL-C(A) in duplicates, and lipoprotein characterization using FPLC (B). (C) Plasma (1 μL) was separated on gels and probed for apoA1 (top) and apoB (bottom) using specific antibodies. Images were quantified using ImageJ and plotted as % of Ctrl.(D) Total plasma (10 μL, left) and HDL (20 μg/mL, right) were used in triplicates to study efflux of 3H-cholesterol from J774 macrophages. (E-F) After 4 months, mice were dissected to visualize plaques in the aortae (E). Total aortas were opened and stained with Oil Red O to visualize lipid deposition (F). *, P<0.05; **, P<0.01; ***, P<0.001; ****, P<0.0001, one way ANOVA, non-parametric. Representative of two experiments.

FIGS. 6A-6C. Triglyceride and phospholipid syntheses are reduced in Huh-7 cells overexpressing miR-541-3p and siZnf101. Huh-7 cells were transfected with (A-B) miR-541-p or antimiR-541-3p (20 nM) or (C-D) different siRNAs (10 nM) in triplicates. After 24 h, cells were washed and incubated with H-glycerol (5 μCi) for 16 h. Lipids were extracted from media using CHCl₃ and CH₃OH. Cells were washed and incubated with isopropanol for 16 h at 4° C. Lipids were evaporated, resuspended in isopropanol, and separated on thin layer chromatography plates. Bands corresponding to triglyceride and phospholipid were excised and counted.

FIG. 7. A schematic diagram depicting the role of miR-541-3p in the control of plasma lipoproteins and atherosclerosis. The present results show that miR-541-3p downregulates ZNF101 and CASZ1 by enhancing post-transcriptional degradation of mRNAs after interacting with their 3′-UTRs. Furthermore, the data indicate that ZNF101 is an enhancer of APOB, and CASZ1 is a repressor of APOA1. Hepatic knockdown (KD) of Zfp961, an orthologue of ZNF101, reduces plasma apoB-containing lipoproteins, whereas KD of Casz1 increases high density lipoproteins in mice. The data show that hepatic KDs of these transcription factors reduces atherosclerosis in mice induced by the expression of mutant PCSK9.

FIGS. 8A-8C. Screening a library to identify microRNAs regulating apoB and apoA1 secretion in human hepatoma Huh-7 cells. (A) Huh-7 cells were reverse transfected in duplicate plates with a human miRDIAN mimic 16.0 library (Dharmacon) of 1237 miRs at 50 nM. After 24 h, cells received complete media with 10% FBS. After another 24 h, cells were incubated with complete media containing 10% FBS and oleic acid/BSA complexes (0.4 mM/1.5%) for 2 h to avoid identification of miRs that affect posttranslational degradation of apoB. Media were used to quantify apoB and apoA1 levels by ELISA. Few wells in each plate were simultaneously transfected with negative control (Ctrl) or miR-30c (positive control, reduces apoB). (B) Percentage change in media apoB and apoA1 in two plates exposed to the same miRs were compared with the negative control. Changes (%) in plate 1 are plotted against plate 2. Correlation between two plates with respect to changes in media apoB and apoA1 was determined. (C) Different miR family members with the same seed sequence showed similar reductions in media apoB and apoA1 indicating internal consistency in the regulation of apoB and apoA1 secretion by family members.

FIGS. 9A-9G. Regulation of apoB and apoA1 by miR-541-3p. (A-C) Full gels probing apoB100 in the (A) media and (B-C) cell lysates of Huh-7 cells treated with miR-541-3p mimics (top) or antimiR-541-3p (bottom), respectively. Bands were quantified by ImageJ and plotted as % of control (insets). The densitometric analysis showed significant reductions in apoB protein levels in cells and media of Huh-7 cells overexpressing miR-541-3p mimics and significant increases in cells overexpressing antimiR-541-3p. MiR-541-3p mimics and antimiR-541-3p had no effect on β-actin protein levels. (D-F) Full gels and quantification of apoA1 bands in media (A) and cells (B) transfected with miR-541-3p mimics and antimiR-541-3p. Protein bands were quantified using ImageJ and normalized to β-actin (C) and plotted as % of control (bottom). MiR-541-3p mimics increase whereas antimiR-541-3p decreases apoA1 expression. (G) Quantification of MTP and ABCA1 mRNA levels in Huh-7 cells transfected with different amounts of miR-541-3p mimics.

FIGS. 10A-10G. Identification of transcription factors (TFs) regulating apoB. (A) Tf2dna database was queried for TFs that could potentially regulate APOB gene expression. This identified 31 potential TFs that could regulate APOB expression. Ten of these TFs were identified as targets of miR-541-3p using Target Scan. (B) Transcript levels of these 10 TFs were quantified in triplicate using specific primers. Seven TFs could be measured in Huh-7 cells. (C) Huh-7 cells were transfected in triplicate with 20 nM miR-541-3p or antimiR-541-3p. After 48 h, changes in mRNA levels were quantified. Znf101 significantly reduced or increased, respectively, in cells transfected with miR-541-3p mimics or antimiR-541-3p. (D) Huh-7 cells were transfected with miR-541-3p mimics (20 nM) or siZnf101 (28 nM), alone or in combination. After 48 h, cell lysates were used to measure different mRNA levels (left). Conditioned media was used to quantify apoB and apoA1 levels by ELISA (right). (E) Target Scan predicted that the 3′-UTR of ZNF101 mRNA contains complementary bases that could pair with miR-541-3p seed sequence (top). Target Scan was used to determine the conservation of Znf101 3′-UTR in different species. Conserved sequences were found in the rhesus monkey and chimpanzee (bottom). (F) Schematic diagrams of plasmids expressing Gaussia-Dura luciferase under the control of Znf101 3′-UTR obtained from Genecopoeia. The plasmid also constitutively expresses alkaline phosphatase under the control of cytomegalovirus (CMV) promoter and is used as a control. The highlighted bases were mutated in the wild-type plasmid. (G) Schematic diagram showing potential Znf101 binding site in the APOB promoter. Transcription start site (TSS) and stop sites are identified. APOB promoter and coding sequences (seq) are shown (not to scale). Potential Znf101 binding site was mutated as shown in red.

FIGS. 11A-11G. Identification of TFs regulating apoA1. (A) Tf2dna database was searched for TFs that could regulate APOA1 gene expression. This identified 59 potential TFs. Target Scan predicted 18 of these TFs to be targets of miR-541-3p. (B) mRNA levels of these TFs were quantified in triplicate by qRT-PCR. Huh-7 cells express 13 of these TFs. (C) Huh-7 cells were transfected with 20 nM miR-541-3p mimics or antimiR-541-3p. After 48 h, changes in mRNA levels of different TFs were quantified in triplicate. Casz1 was significantly decreased and increased, respectively, in cells expressing miR-541-3p mimics or antimiR-541-3p. (D) Huh-7 cells were transfected in triplicate with miR-541-3p mimics (20 nM) or siCasz1 (28 nM), alone or in combination. After 48 h, mRNA levels were quantified in cells (left), and protein levels in conditioned media (right) in triplicate. MiR-541-3p mimics and siCasz1, individually and combined, increased apoA1 levels to a similar extent indicating that they are in the same pathway. (E) Target Scan 7.2 showed that Casz1 3′-UTR contains a complementary sequence that could base pair with miR-541-3p seed sequence (top). Clustal W alignment algorithms indicated conservation of the 3′-UTR of Casz1 in primates (bottom). (F) Plasmids expressing luciferase with Casz1 3′-UTR were obtained from Genecopoeia. Casz1 3′-UTR was mutated as shown below in red. (G) Schematic diagram showing potential Casz1 binding site in the APOA1 promoter (not to scale). Potential binding site sequence was mutated as shown in red.

FIGS. 12A-12E. Genome wide associations of variants in the M/R541, CASZ1, and ZNF101 loci with plasma lipids and lipoproteins. (A-C) Association results for SNPs (−log 10 P value, y-axis) as a function of genomic coordinates using human gene version 19 (hg19) for (A) M/R541, (B) CASZ1, and (C) ZNF101 gene loci with plasma total cholesterol, LDL, apoB, HDL-C, apoA1, and triglycerides in the UK Biobank using the Pan-UKB project summary statistics. The bottom panel shows genes at each locus, as annotated in the UCSC Genome Browser. The most highly associated SNPs are represented as purple diamonds, and the linkage disequilibrium (LD) values (1000 Genomes data) are in the inset. Light blue dotted lines indicate estimated recombination hotspots. Forrest plot on the right panels show the associations (beta±standard error) of the top associated SNP with plasma lipids and apolipoproteins levels. (*) Correspond to genome wide significant p-values (p<5.0E-08). Raw data are presented in Supplemental Table 3. (D-E) (D) Forrest plots show the associations (beta±standard error) of the top associated SNP with plasma lipids and apolipoproteins levels in GLGC. (E) LocusCompare plot shows the relationship between SNPs in the CASZ1 locus (±100 kb) and their association with total cholesterol plasma levels in the x-axis with the gene expression of CASZ1 in the liver (eQTL_liver-log 10(P) from GTEx dataset V7) in the y-axis.

FIGS. 13A-13B. Mouse orthologs of human genes. (A) Identification of mouse orthologs of human Znf101 and Casz1 and their role in the regulation of mouse apoB and apoA1 expression. Mouse liver AML12 cells were transfected in triplicate with different concentrations of mouse siCasz1 (left), siZfp101 (middle) or siZfp961 (right). After 48 h, the indicated mouse mRNAs were quantified in triplicate. These studies identified Casz1 and Zfp961 as functional mouse orthologs of human CASZ1 and ZNF101 genes. * P<0.05; ** P<0.01; ***P<0.001; ****P<0.0001. (B) Male C57Bl6 mice (n=4, 5 months old) were fed chow (CD), Western (WD) or obesogenic (HFD) diets for 13 weeks. Livers were collected to measure mRNA levels. Zfp961 expression increased in the livers of high fat diet fed mice. One-way ANOVA, * P<0.05, **P<0.001.

FIGS. 14A-14E. Effect of hepatic Casz1 and Zfp961 knockdown on different parameters in mice. Mice (C57Bl6J, female, 2.5-month-old) were transduced with AAV8 (2.5×10¹¹ gc/mouse) expressing shControl (shCtrl, n=4), shCasz1 (n=4) or shZfp961 (n=4), alone or combined (sh(C+Z), n=5), and started on a Western diet. (A) After 6 weeks, livers were collected to measure in triplicate different TFs and lipid metabolism genes. KD of different genes had no effect on their expression (top). Western blot analysis was performed to measure changes in MTP and ABCA1 (bottom). No significant differences in the expression of MTP and ABCA1 were found amongst different groups. (B) After 6 weeks, lipids were extracted from liver slices and triglyceride and cholesterol levels were measured in triplicate and normalized with protein levels. KD of different genes had no effect on hepatic lipids. (C) Plasma was used to measure ALT and AST activities in triplicate. These enzyme activities were unaffected by KD of Casz1 or Zfp961. (D) Weight gain in mice was monitored over the course of 6 weeks. No significant differences were observed. (E) After 5 week of injections, mice were placed in CLAMS to measure different physiological parameters. No significant differences were noted.

FIGS. 15A-15C. Effect of knockdown of different TFs on atherosclerosis. All mice received AA8 expressing mutant mouse PCSK9. In addition, mice were transduced with shCtrl, shCasz1, shZnf101, or shCasz1+shZnf101, and started on a Western diet. (A) After 4 months, livers were collected to measure mRNA levels in triplicates. (B) Detection of plasma apoA1 (top) by western blotting. Plasma (1 μL) was separated on a 10% gel, transferred, and probed with anti-apoA1 antibodies. Plasma (1 μL) was separated and stained with Coomassie blue (bottom) for control. (C) Detection of apoB by western blotting (top) after separating plasma (1 μL) on a 6% gel. Total plasma was separated and stained with Coomassie blue (bottom) for control.

FIGS. 16A-16E. Effect of knockdown of different TFs on physiological parameters. Mice were transduced with adenoviruses expressing gain-of-function mutant Pcsk9 and those expressing different shRNAs as in FIG. 12. (A) After 4 months, livers were collected to measure TFs and lipid metabolism genes in triplicates (top). Western blot analyses were performed to measure changes in MTP and ABCA1 proteins levels (bottom). MTP and ABCA1 mRNA and protein levels were similar in all the groups indicating that KD of different TFs had no effect on their expression. (B) Lipids were measured in triplicates in livers and normalized to protein levels. Knockdown of different TFs had no effect on hepatic lipids. (C) Plasma was used to measure AST/ALT activities in triplicates. (D) At the end, different tissues were collected. Total body and adipose tissue weights in different knockdown mice were lower than in control mice (right). No differences in other organ weights were observed. (E) After 3 months, mice were placed in CLAMS (comprehensive laboratory animal monitoring system) to monitor physiological indices.

FIGS. 17A-17B. Effect of knockdown of different TFs on atherosclerosis. Mice were transduced with adenoviruses expressing gain-of-function mutant Pcsk9 and those expressing different shRNAs as in FIG. 12. (A) Aortas with major arteries were dissected from all mice, photographed, and presented as a collage. (B) Whole aortas were stained with Oil Red O and all images were compiled.

FIGS. 18A-18D. Hepatic knockdown of Casz1 and Znf961 has no effect on the mRNA levels of genes involved in β-oxidation, but reduces the expression of genes in lipogenesis. Mice were transduced with viruses as in FIG. 11 (A, C) and FIG. 12 (B, D). Livers from these mice were used to quantify in triplicate different mRNAs involved in β-oxidation (A, B) and lipogenesis (C, D).

### DETAILED DESCRIPTION

Unless defined otherwise herein, all technical and scientific terms used herein have the same meaning as commonly understood by one of ordinary skill in the art to which this disclosure pertains. The disclosure includes the following abbreviations: ApoA1, apolipoprotein A1; ApoB, apolipoprotein B; ApoB-Lps, apoB-containing lipoproteins; GWAS, genome wide association studies; HDL, high density lipoproteins; KD, knockdown; LDL, low density lipoproteins; miRs, microRNAs; UTR, untranslated region;

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The disclosure includes all polynucleotide sequences described herein expressly and by reference, and every polynucleotide sequence referred to herein includes its complementary sequence, and its reverse complement. All segments of polynucleotides from 10 nucleotides to the entire length of the polynucleotides, inclusive, and including numbers and ranges of numbers there between are included. All nucleotide sequences associated with any database accession numbers are incorporated herein by reference as they exist in the database as of the date of the filing of this application or patent. The disclosure includes all polynucleotide sequences described herein expressly or by reference that are between 80.0% and 99.9% identical to the described sequences.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another example includes from the one particular value and/or to the other particular value. Similarly, when values are expressed as approximations, by the use of the antecedent “about” or “approximately” it will be understood that the particular value forms another example. The term “about” and “approximately” in relation to a numerical value encompass variations of +/−10%, +/−5%, or +/−1%.

Any result obtained by using an miRNA or other agent as described herein can be compared to a suitable control. In examples, the control is a result from cells that have not received the miRNA or other described agent.

miRs, small (˜22 nucleotides) non-coding RNAs, bind via their seed sequences to the 3′-untranslated regions (3′-UTRs) of target mRNAs to either degrade them or to decrease translation (12, 13). MiRs regulate many genes; therefore, miR mimics, or their inhibitors, anti-miRs, can be used to understand regulation of different interrelated biological pathways. In this disclosure multiple miRs were analyzed to determine if they could modulate plasma lipoproteins by reducing apoB and increasing apoA1. The disclosure reveals that a primate-specific miR, miR-541-3p, reduces apoB and increases apoA1 secretion in human hepatoma cells. Mechanistic studies led to identification of transcription factors, Znf101/Zfp961 and Casz1 which act as an enhancer and a repressor of apoB and apoA1, respectively. The disclosure demonstrates that hepatic knockdown of these transcription factors alters plasma lipoprotein profile and reduces atherosclerosis in mice. The disclosure also demonstrates similar effects using miR-541-3p and synthetic mimics thereof.

In examples, an miRNA used in this disclosure is miRNA-541-3P, which comprises or consists of the sequence UGGUGGGCACAGAAUCUGGACU (SEQ ID NO:1). Precursors of miRNA-541-3P are known, and uses of the precursors are encompassed by the disclosure.

In examples, SEQ ID NO:1 comprises one or more modifications. In examples, a modification comprises a 2′-deoxy-2′-fluoro (2′-F) ribosugar modification, a 2′-O-methyl (2′-OMe) ribosugar modification, a 2′-a GalNAc clicked cytidine, or a phosphorothioate linkage. The disclosure also includes use of modified ribonucleotides or deoxyribonucleotide, and thus include RNA/DNA hybrids. In non-limiting examples, modified ribonucleotides may comprise methylations and/or substitutions of the 2′ position of the ribose moiety with an —O— alkyl group containing 1-6 saturated or unsaturated carbon atoms, or with an —O-aryl group having 3-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In examples modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof.

In examples, a therapeutically effective amount of one or more described agents is delivered to an individual. The term “therapeutically effective amount” as used herein refers to an amount of a described miRNA or other described agent, in a single dose or multiple doses, to achieve the intended purpose of treatment. The amount desired or required may vary depending its mode of administration, patient specifics and the like. Appropriate effective amounts can be determined by one of ordinary skill in the art informed by the instant disclosure using routine experimentation. In examples, a therapeutically effective amount of a described agent is administered to an individual in need thereof. In examples, a described agent is administered to an individual using an expression vector such as a viral vector. The disclosure includes the proviso that any described agent can be administered in a viral vector free manner. In examples, a combination of described agents is administered to an individual. In examples, a described agent alone or in combination with at least one other described agent is administered to an individual and is sufficient to achieve a therapeutic effect. The described agent(s) may also be used prophylactically.

In examples an agent described herein may be administered as a double stranded polynucleotide complex. In non-limiting examples, RNAi-mediated silencing and/or reducing mRNA encoding at least one of ZNF1010r CASZ1 is performed. The genomic coding sequence, mRNA sequence, and amino acid sequences of human ZNF101 and CASZ1 are known in the art, as are their corresponding sequences in mouse, wherein Zfp961 is an orthologue of human ZNF101. It is considered that results presented in this disclosure using Zfp961 are predictive of results that modulate human ZNF101.

In examples, RNAi-mediated silencing of a described gene is performed by delivery of any suitable RNAi agent. In examples, an siRNA-based approach is used. This can be performed by introducing and/or expressing one or more suitable short hairpin RNAs (shRNA) in the cells. shRNA is an RNA molecule that contains a sense strand, antisense strand, and a short loop sequence between the sense and antisense fragments. shRNA is exported into the cytoplasm where it is processed by dicer into short interfering RNA (siRNA). siRNA are 21-23 nucleotide double-stranded RNA molecules that are recognized by the RNA-induced silencing complex (RISC). Once incorporated into RISC, siRNA facilitate cleavage and degradation of targeted mRNA. Thus, for use in RNAi mediated silencing or downregulation of expression of a described protein, siRNA, shRNA, or miRNA can be used. In alternative examples, a functional RNA, such as a ribozyme is used. In examples, the ribozyme comprises a hammerhead ribozyme, a hairpin ribozyme, or a Hepatitis Delta Virus ribozyme. In examples, the RNAi agent may be modified to improve its efficacy, such as by being resistant to nuclease digestion.

In examples a chromosome within a cell is modified to decrease expression of a described gene. In examples, a genome of one or more cells as described herein may be modified to disrupt or delete a described gene. This approach can be performed using any designer nuclease, such as those that are functional in any CRISPR system. In examples, the nuclease is an RNA-guided CRISPR nuclease. A variety of suitable CRISPR nucleases (e.g., Cas nucleases) are known in the art. In non-limiting examples, the Cas comprises a Cas9, such as Streptococcus pyogenes (SpCas9). Derivatives of Cas9 are known in the art and may also be used. Such derivatives may be, for example, smaller enzymes than Cas9, and/or have different proto adjacent motif (PAM) requirements. In a non-limiting examples, the Cas enzyme may be Cas12a, also known as Cpf1, or SpCas9-HF1. In examples a Cas3 enzyme or a transposon coupled to a nuclease may be used.

In examples, a nuclease may be administered directly to the cells. For example, a Cas protein and suitable guide RNA(s) may be administered to the cells as ribonucleoproteins (RNPs) using any suitable technique. Alternatively, the Cas protein may be introduced into the cells separately from the guide RNAs. In examples, a viral expression vector may be used to introduce sequences encoding one or more of the described nucleases and/or related effector complex proteins into the cells, or to express a described miRNA. In examples, and adeno viral vector or adeno-associated viral vector may be used. As an alternative to a Cas system, a zinc finger nuclease, or a transcription activator-like effector nuclease (TALEN), or a transposon-based DNA editing system can be used to modify cells as described herein.

In examples, the disclosure provides for inhibiting the function of a described protein encoded by a described gene. In examples the function of a protein is inhibited using one or more binding partners. The binding partner may be any form of an antibody that specifically binds to a described protein, including but not limited to an intact antibody, a bispecific antibody, a multispecific antibody, an antigen-binding (Fab) fragment, an Fab′ fragment, an (Fab′)2 fragment, an Fd, an Fv, a single domain fragment or single monomeric variable antibody domain, a single-chain Diabody (scDb), a single-chain variable fragment (scFv), a CrossMab format, a tri-specific binding partner, a DARPin, an affibody, or an affimer. In examples, the function of a described protein may be inhibited using a peptide or small molecule drug. “Agents” as used herein include but are not necessarily limited RNAi agents, gene editing systems, binding partners, and small molecule drugs. In examples one or more agents described herein may be combined with other agents that are intended for prophylaxis or treatment of a described disorder or any condition or symptom for which an individual is in need of prophylaxis or therapy.

In one example, expression of a protein described herein can be increased by providing to cells an mRNA encoding the protein, or an expression vector encoding the protein.

In an example, a prophylactic or therapeutic agent, such as RNA or other described polynucleotides or derivatives thereof, is provided in combination with any suitable nucleic acid delivery agent, non-limiting examples of which are described in Mitchell, M. J., et al., Nat Rev Drug Discov. 20, 101-124 (2021), the description of which is incorporated herein by reference.

In non-limiting embodiments a polynucleotide of this disclosure is combined with one or more unilamellar and/or multilamellar vesicular structures such as liposomes or lipid nanoparticles, cationic polymers, lipoplexes, polyplexes, or inorganic nanoparticles. Any delivery agent described herein may comprise polyethylene glycol (PEG) and thus may be PEGylated.

In examples, introducing a described polynucleotide into cells and/or inhibiting expression or function of ZNF101 and/or CASZ1 reduces lipogenesis and/or production of TG rich lipoproteins and atherosclerosis without causing hepatic lipid accumulation. In examples, introducing a described polynucleotide into cells and/or inhibiting expression or function of ZNF101 and/or CASZ1 is such that expression of apoB is decreased and/or expression of apoA1 is increased.

In examples, the disclosure provides for increasing production of endogenously encoded miRNA-541-3P.

In examples, the cells into which a described polynucleotide or other agent as described herein is introduced comprise human liver cells, which may include hepatoma cells. In examples, the cells are within a human individual. In examples, the human individual needs treatment or prophylaxis of a cardiovascular and/or circulatory disorder, such as atherosclerosis. In examples, the individual is in need of plasma lipid reduction.

The following Examples are intended to illustrate but not limit the disclosure.

### Example 1

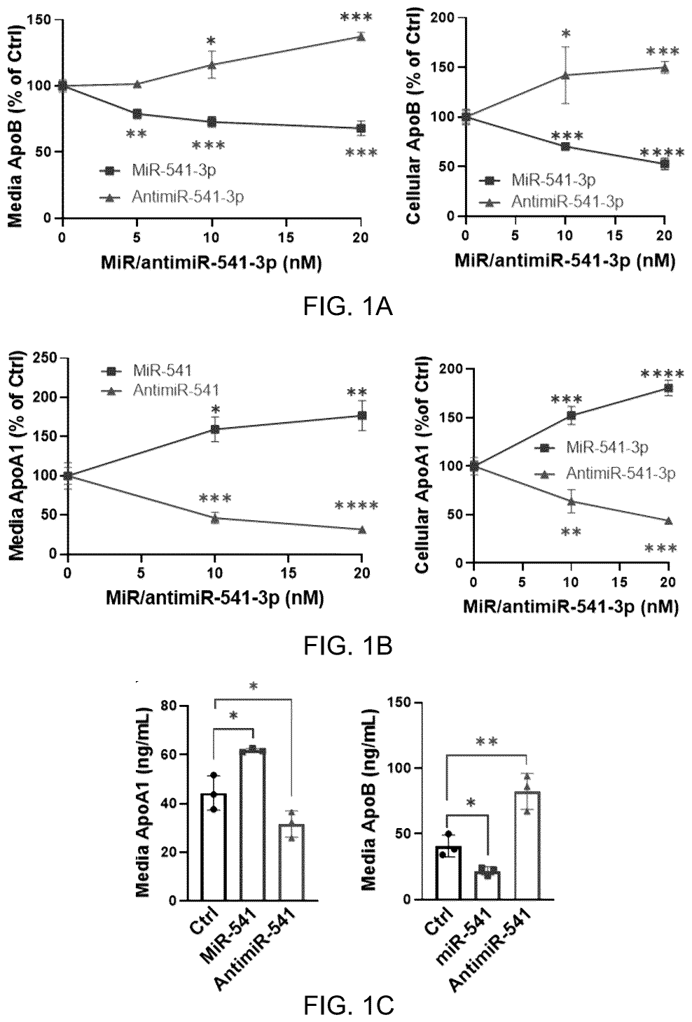

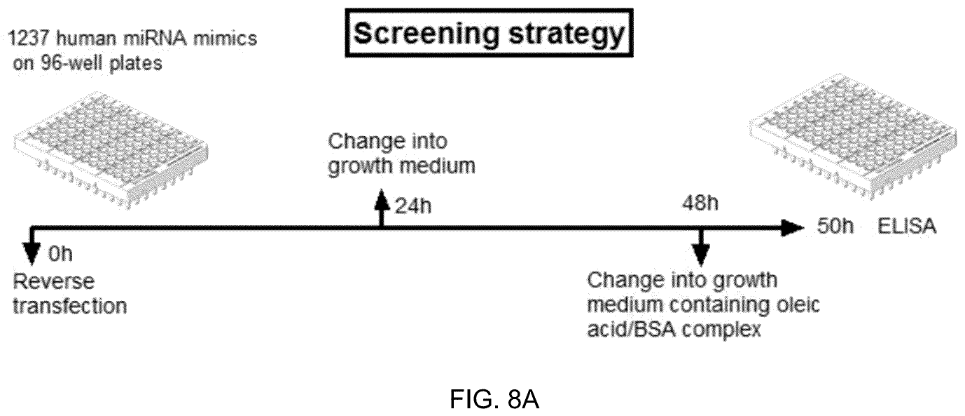

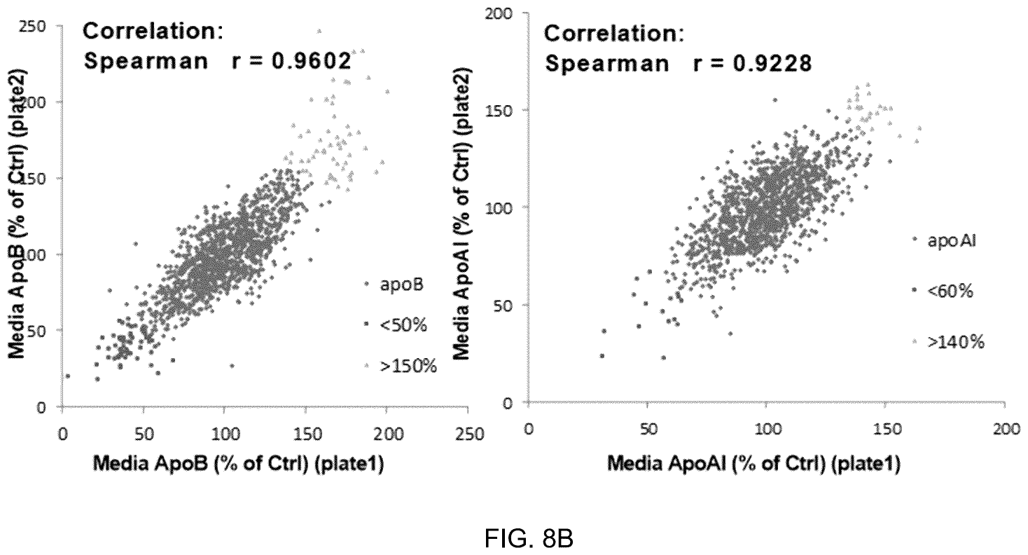

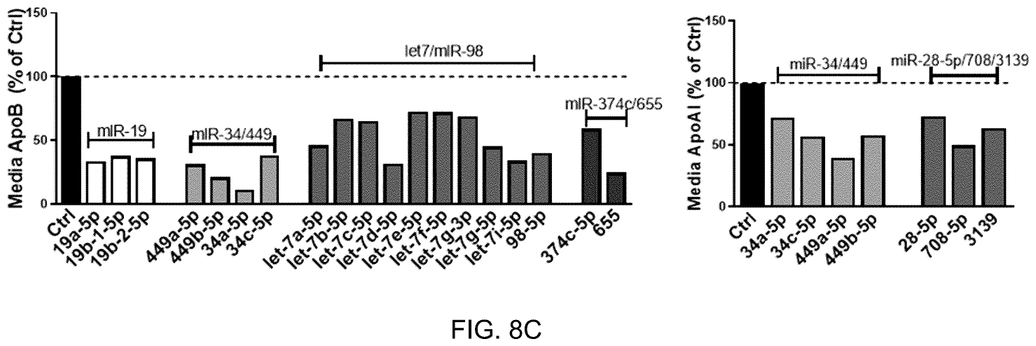

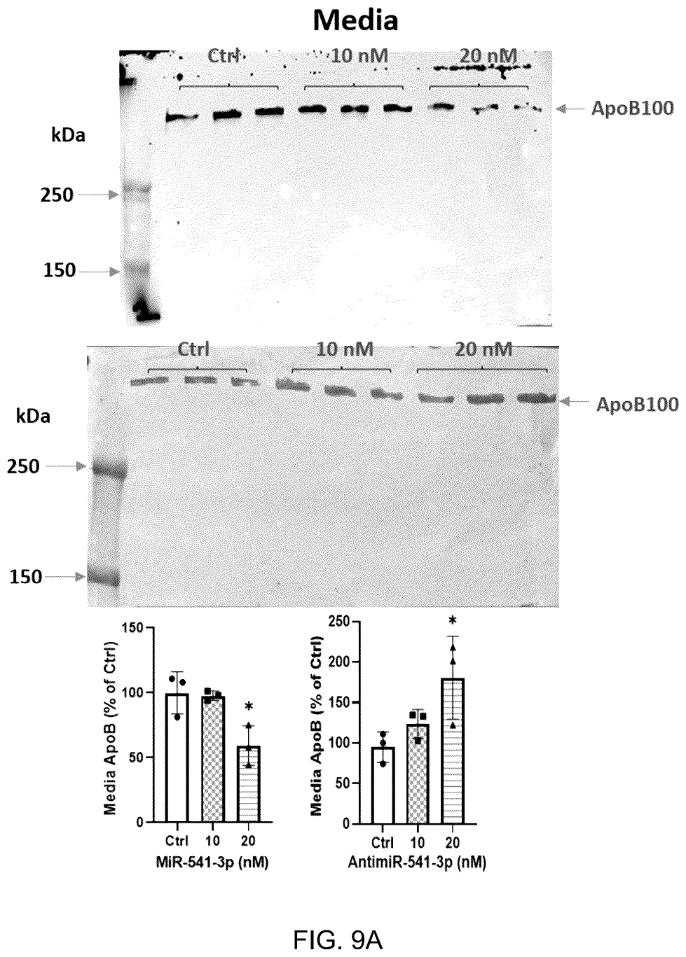

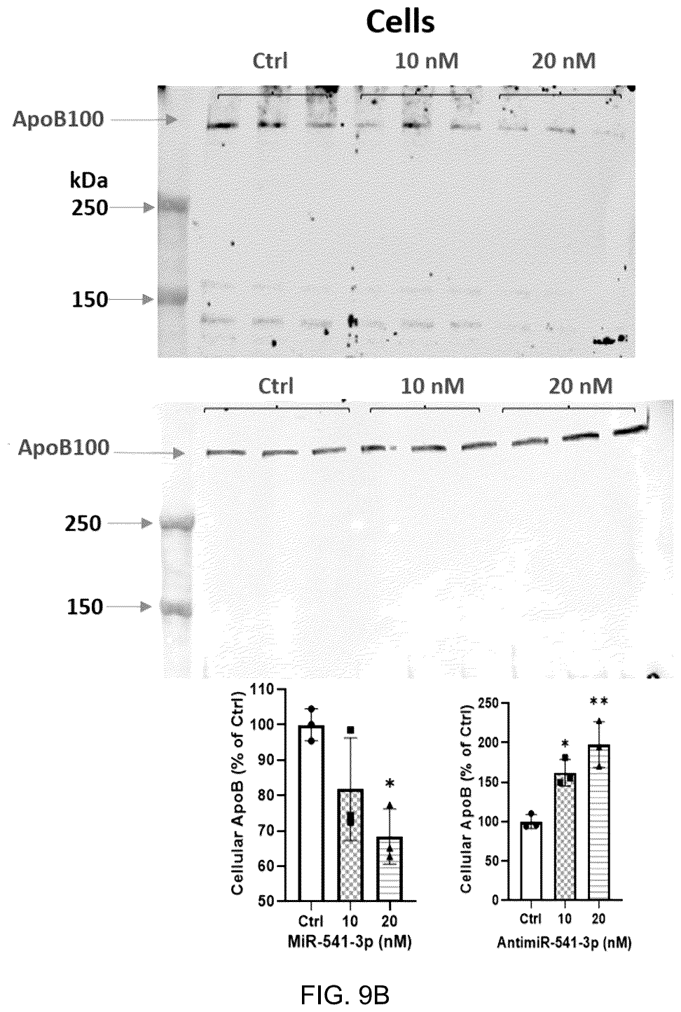

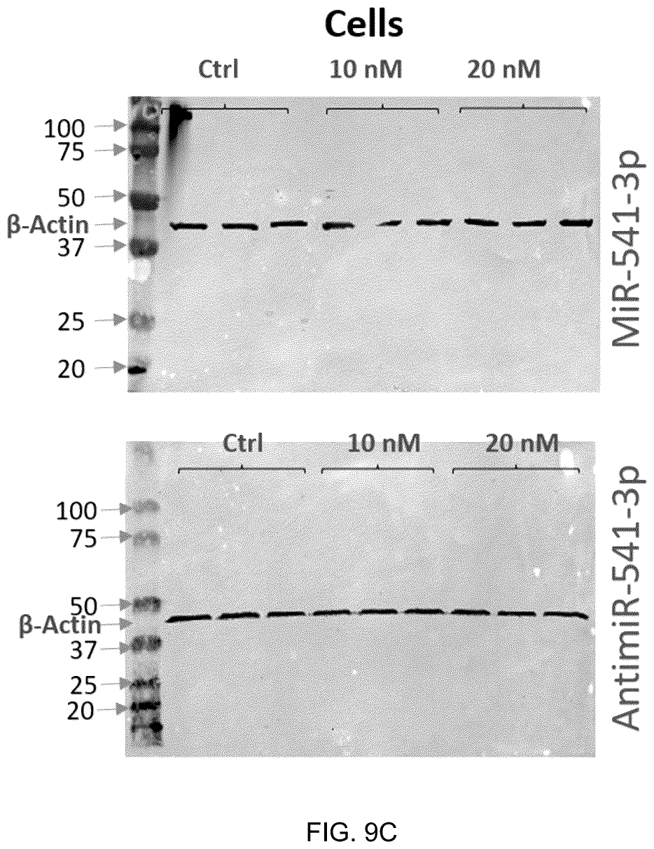

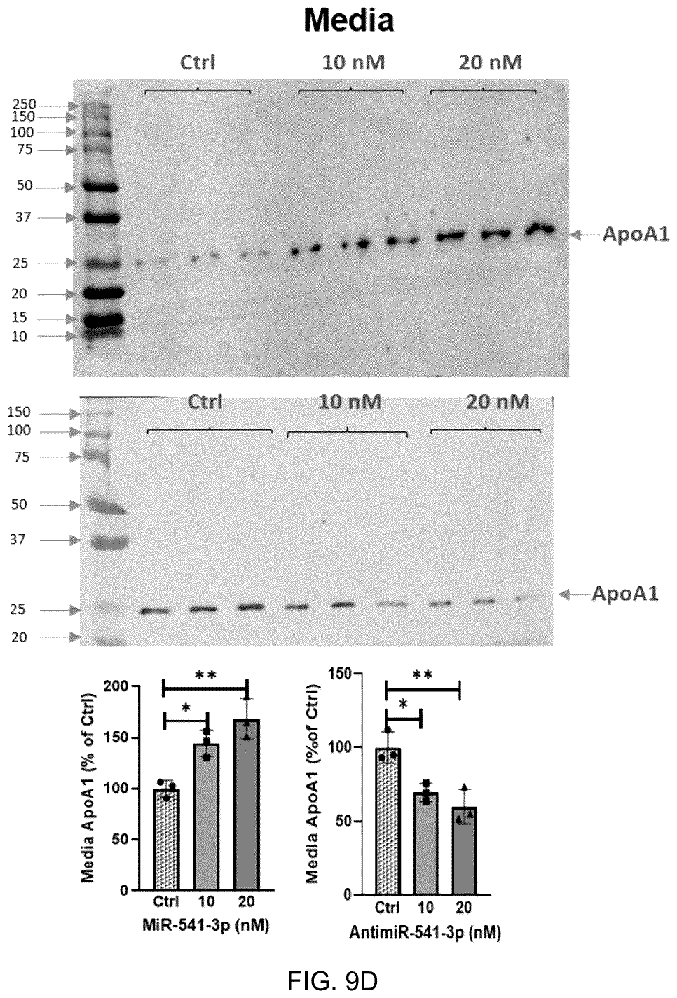

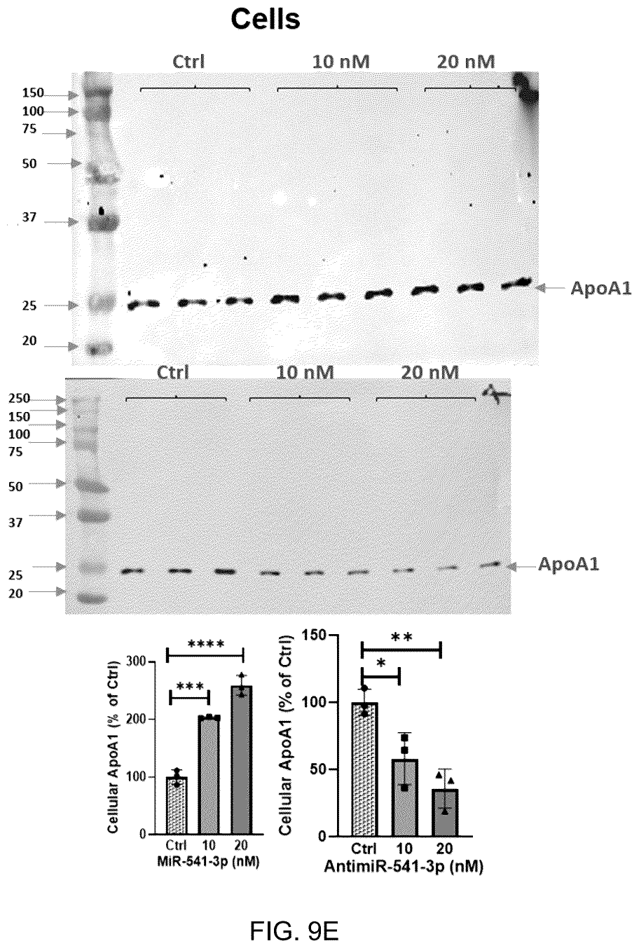

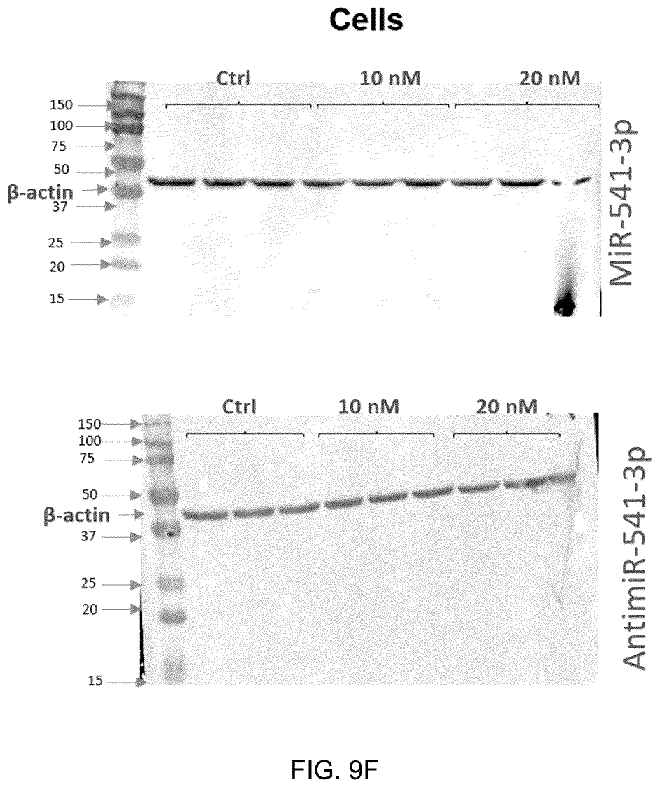

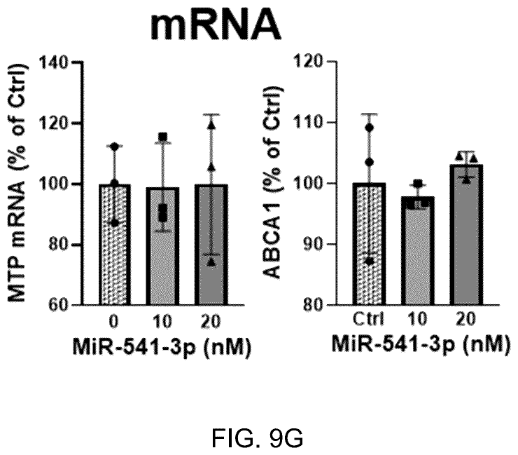

MiR-541-3p reciprocally regulates apoB and apoA1 production We transfected human miR mimic library into human hepatoma Huh-7 cells and quantified secreted apoB and apoA1 (FIG. 8A, Supplementary Table 1). The screening performed in duplicate plates showed high correlation (FIG. 8B) and good internal reproducibility as different members of the same miR families that share the same seed sequence similarly reduced apoB and apoA1 secretion (FIG. 8C). Additionally, miR-30c-5p and miR-33a-5p reduced apoB and apoA1, respectively, (Supplementary Table 1). miR-541-3p was analyzed because it decreased apoB secretion by 58±9%, and increased apoA1 secretion by 95±3%. Increasing concentrations of miR-541-3p and antimiR-541-3p significantly decreased and increased, respectively, media and cellular apoB in dose-dependent manner, as measured by ELISA and Western blotting (FIG. 1A, FIG. 9A-C). In contrast, miR-541-3p significantly increased secreted and cellular apoA1 in a dose-dependent manner, whereas antimiR-541-3p decreased apoA1 protein (FIG. 1B, FIG. 9D-F). Similar reciprocal modulation of apoB and apoA1 secretion was observed in human primary hepatocytes (FIG. 1C). MiR-541-3p had no effect on MTTP or ABCA1 mRNA levels, proteins critical for apoB-Lps and HDL biosynthesis (FIG. 9G). This example therefore shows that miR-541-3p simultaneously and reciprocally regulates apoB and apoA1 production.

### Example 2

MiR-541-3p Mimics Decrease apoB Production by Reducing Znf101 Expression

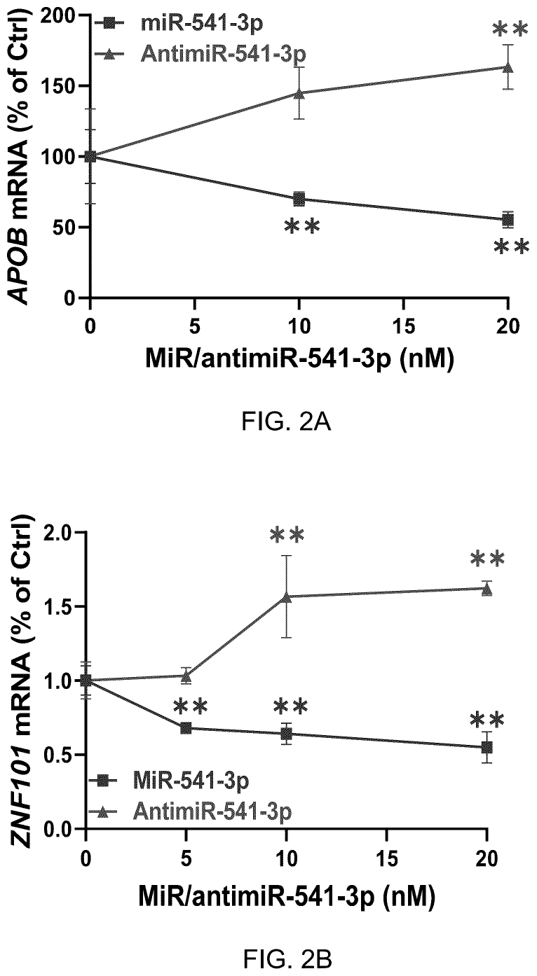

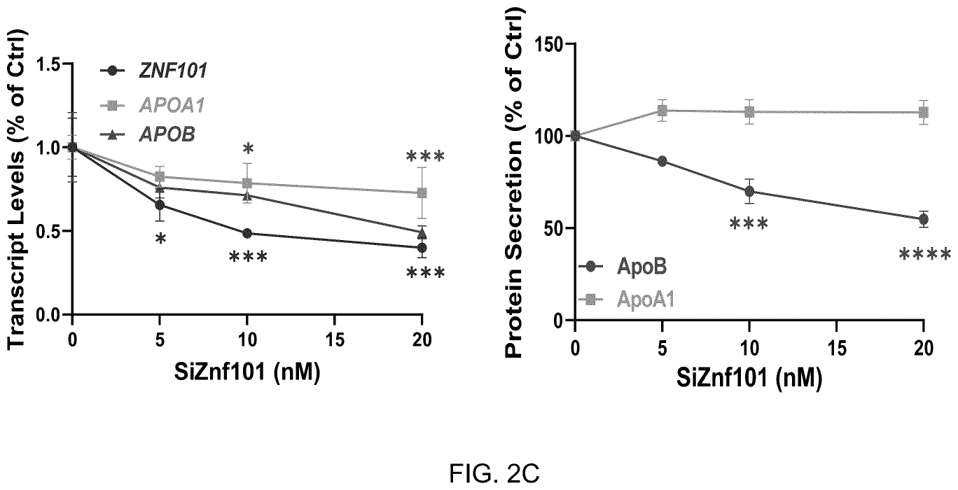

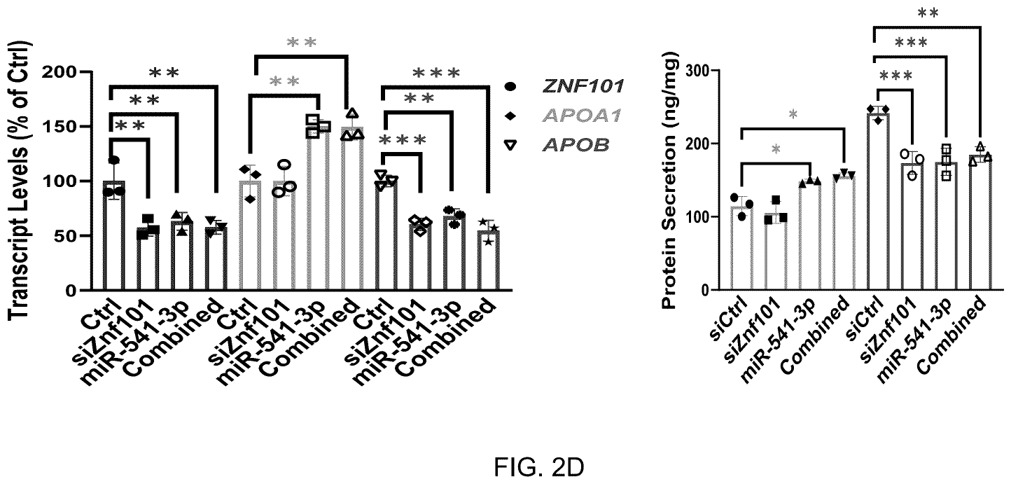

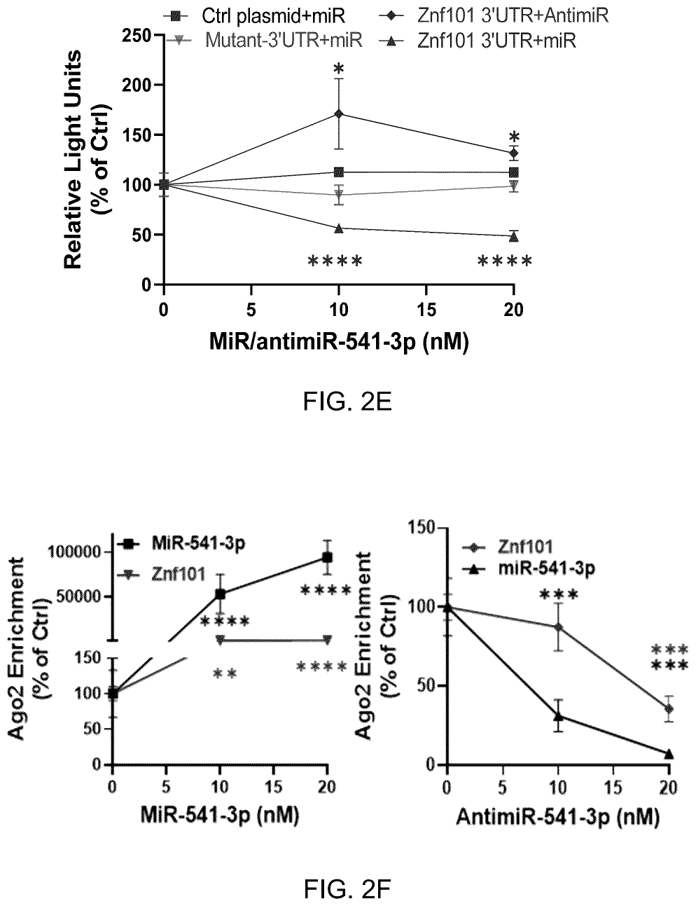

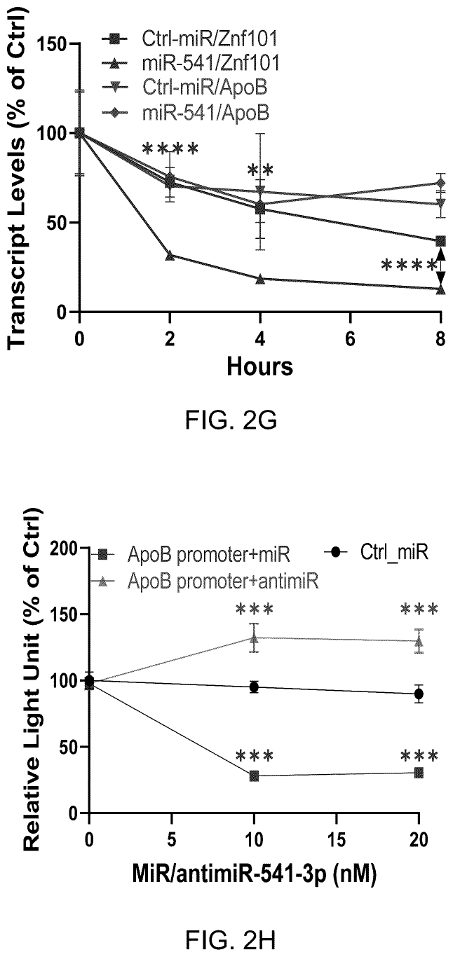

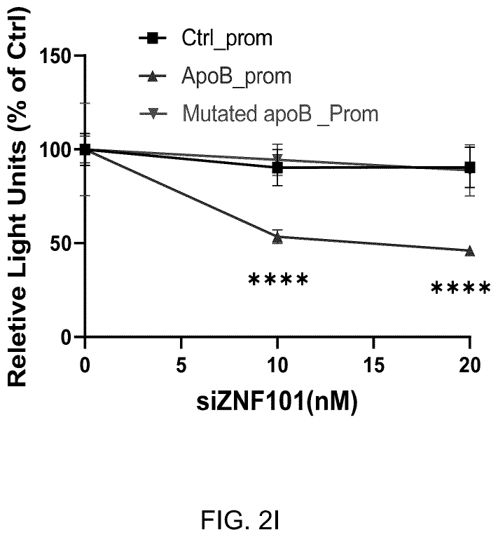

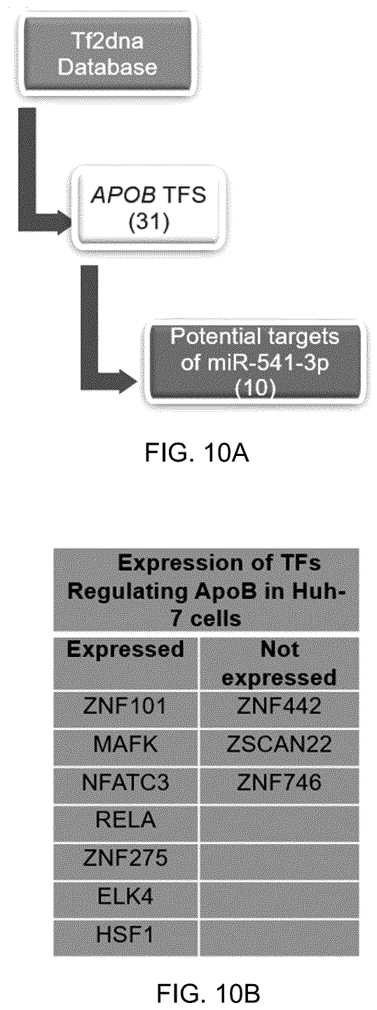

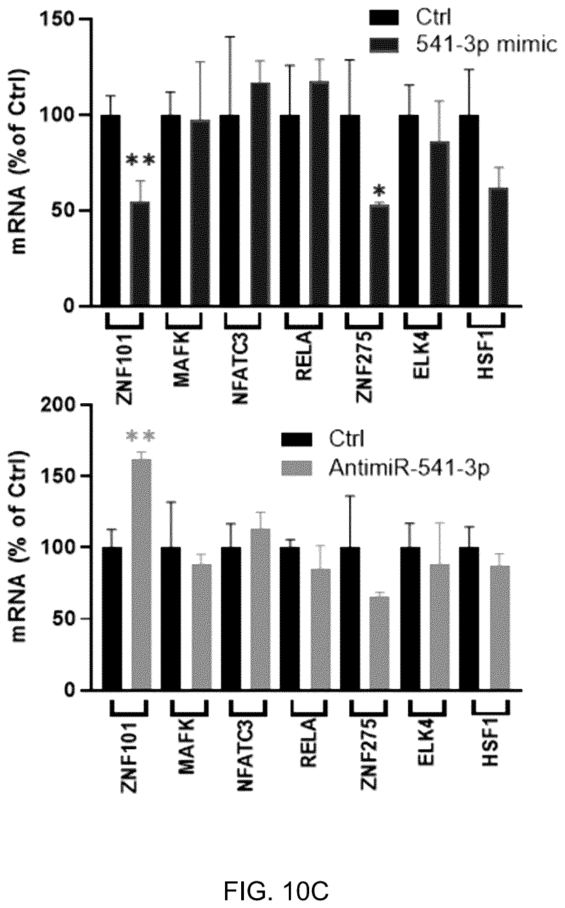

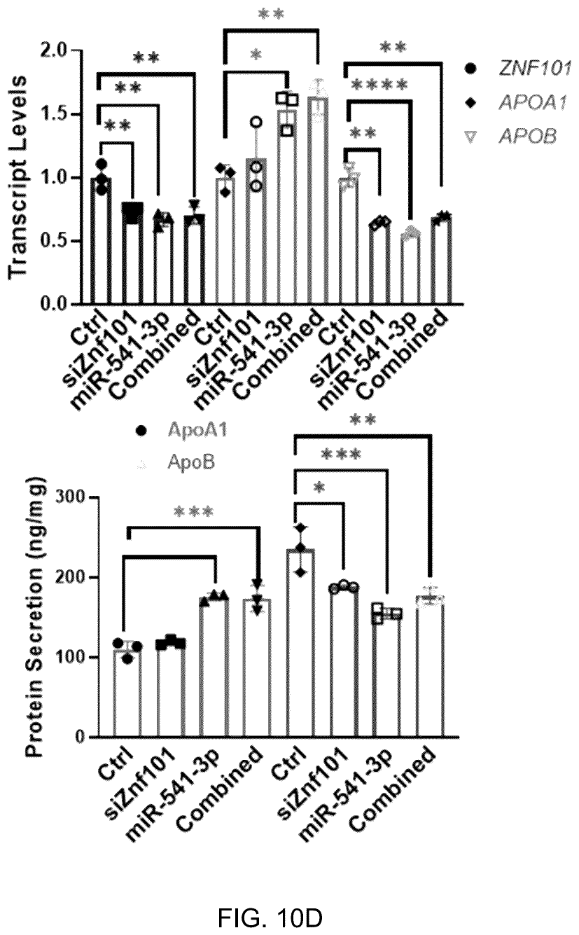

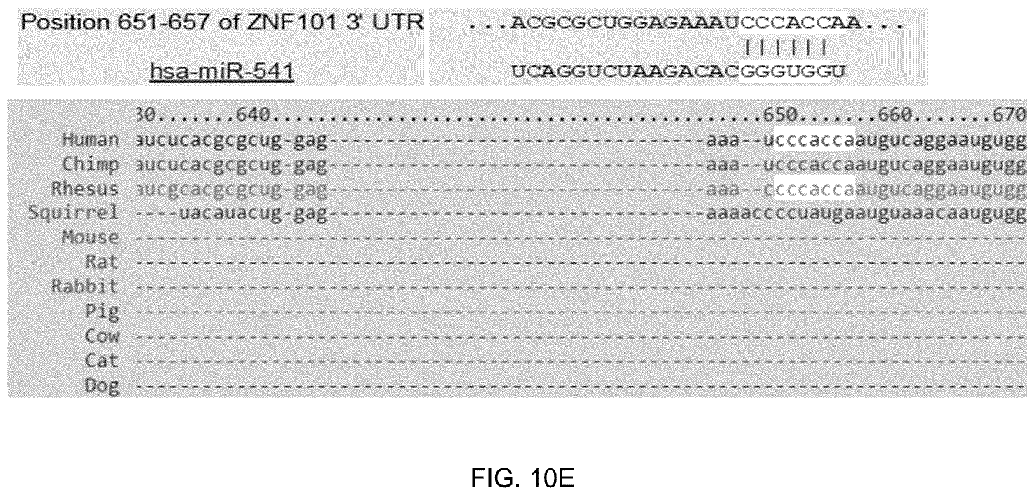

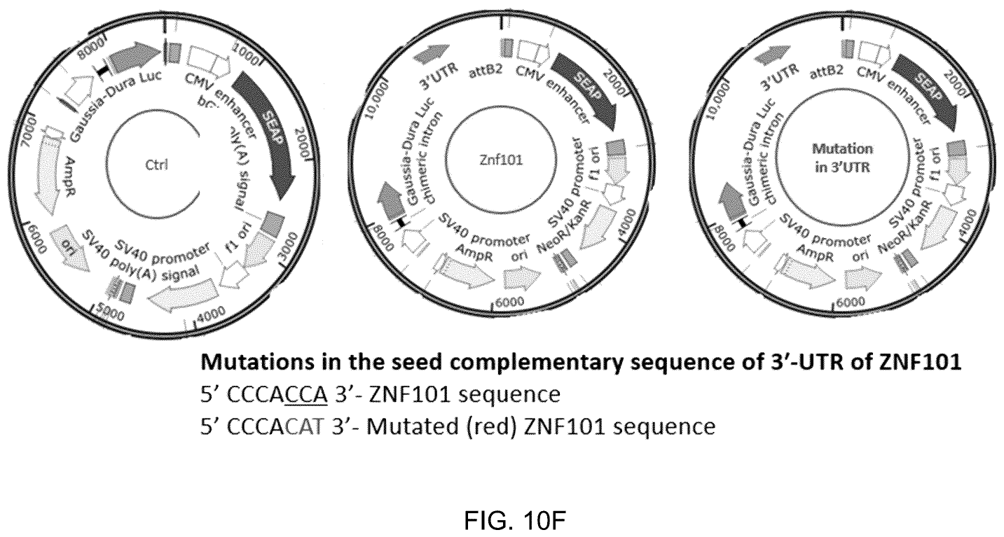

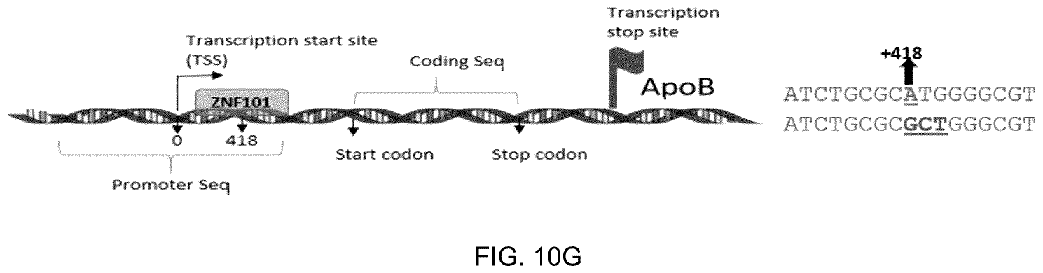

To analyze how miR-541-3p regulates apoB expression, we transfected Huh-7 cells with miR-541-3p mimic and antimiR-541-3p and quantified mRNA levels. MiR-541-3p mimics decreased APOB mRNA, while anti-miR-541-3p had the opposite effect (FIG. 2A). We determined that miR-541-3p is not predicted to pair with the 3′-UTR of APOB mRNA or APOB promoter sequences. Therefore, we analyzed whether miR-541-3p could indirectly modulate mRNA by targeting transcription factors regulating APOB expression. We identified seven transcription factors that could regulate apoB expression, are targets of miR-541-3p, and are expressed in Huh-7 cells (FIG. 10A-B). Notably, miR-541-3p mimics significantly decreased the expression of ZNF101 mRNA, while antimiR-541-3p increased it (FIG. 2B, FIG. 10C). Reducing Znf101 with siZNF101 significantly decreased apoB transcript levels and protein secretion without affecting apoA1 (FIG. 2C). The expression of miR-541-3p mimics, independently or combined with siZNF101, revealed that both miR-541-3p and Znf101 regulate apoB through the same pathway (FIG. 2D, FIG. 10D). These studies indicated that miR-541-3p regulates Znf101 to decrease apoB expression.

To analyze how miR-541-3p regulates Znf101, we investigated whether miR-541-3p interacts with the 3′-UTR of ZNF101 mRNA and induces its degradation. To test this we used four approaches. First, we observed that the ZNF101 mRNA 3′-UTR contains a miR-541-3p interacting site conserved in primates (FIG. 10E). Second, we asked whether miR-541-3p interacts with Znf101 3′-UTR. To answer this, we obtained a plasmid for the expression of luciferase with 3′-UTR from Znf101 mRNA. Znf101 3′-UTR activity was decreased and increased with increasing concentrations of miR-541-3p and anitmiR, respectively (FIG. 2E). When, we mutated the 3′-UTR of ZNF101, we found that miR-541-3p mimics were unable to decrease luciferase expression (FIG. 2E, FIG. 10F). Third, we determined whether miR-541-3p interacts with Znf101 mRNA in Ago2 complexes. Expression of miR-541-3p mimics enriched miR-541-3p and ZNF101 mRNA, while expression of antimiR-541-3p depleted their levels in Ago2 complexes (FIG. 2F). Fourth, we treated cells with actinomycin D to inhibit gene transcription and studied post-transcriptional mRNA degradations in miR-541-3p mimic treated cells. Post-transcriptional degradation of ZNF101 mRNA was faster in cells transfected with miR-541-3p mimics compared to controls, while APOB mRNA decay was similar in cells transfected with either miR-541-3p mimics or the control miR mimics (FIG. 2G). These studies indicate that miR-541-3p enhances post-transcriptional degradation of Znf101, and that there is a temporal delay in the regulation of apoB mRNA. Thus, miR-541-3p likely interacts with the 3′-UTR of ZNF101 mRNA in Ago2 complexes to induce post-transcriptional degradation.

Next, we analyzed how Znf101 regulates apoB. A potential Znf101 binding site in the APOB promoter exists at +418 bp after the transcription start site (FIG. 10G). Luciferase activity under control of the APOB-promoter decreased after the expression of miR-541-3p mimics, but increased with antimiR-541-3p expression (FIG. 2H). Furthermore, siZnf101 reduced APOB-promoter luciferase activity (FIG. 2I), but not when the Znf101 binding site was mutated, indicating that Znf101 is an enhancer of APOB.

### Example 3

MiR-541-3p Mimics Increase apoA1 Production by Reducing Casz1 Expression

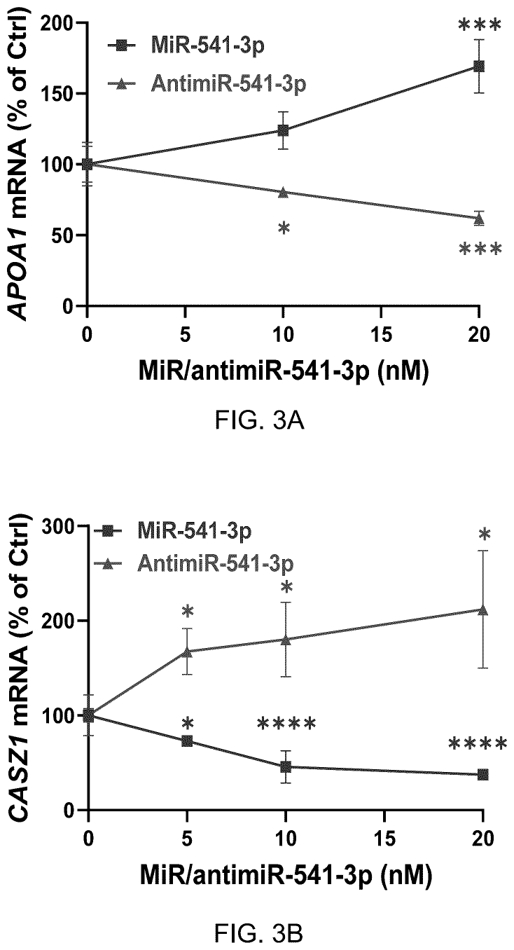

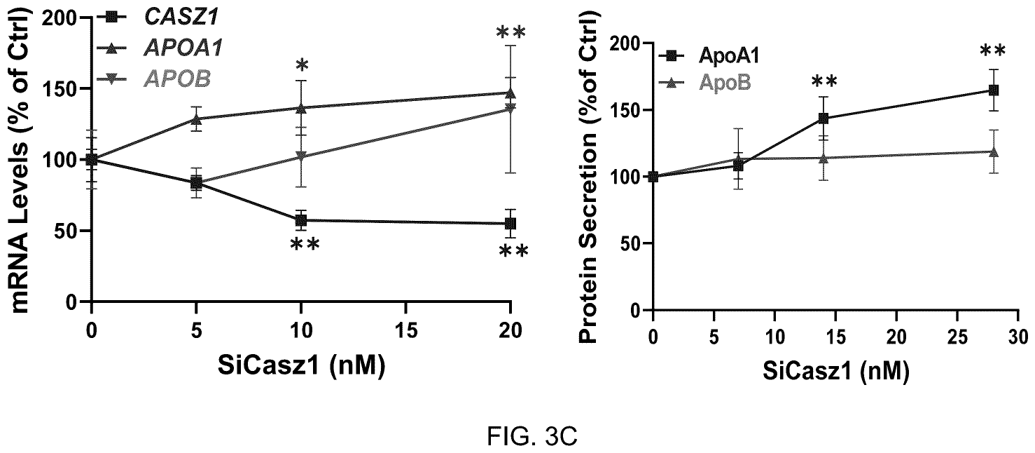

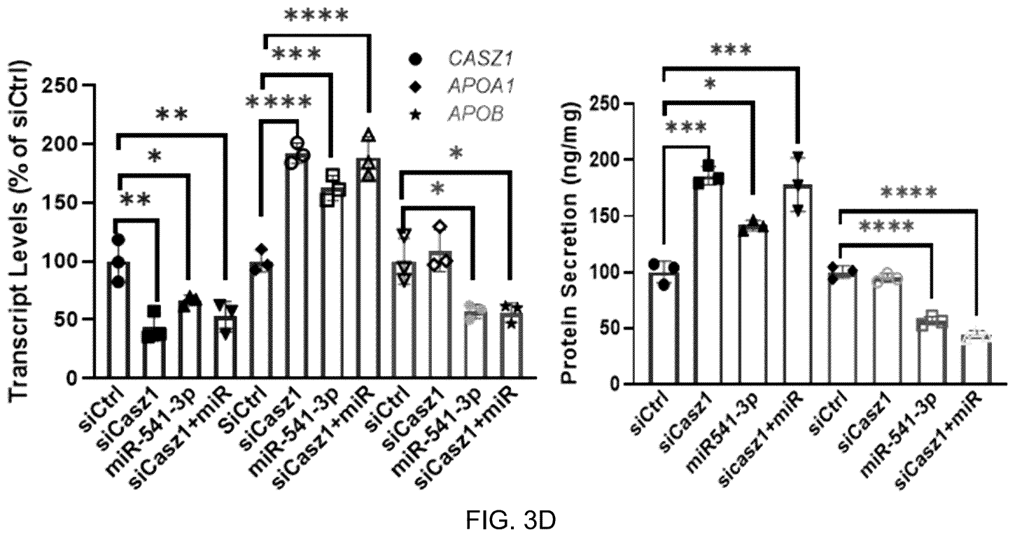

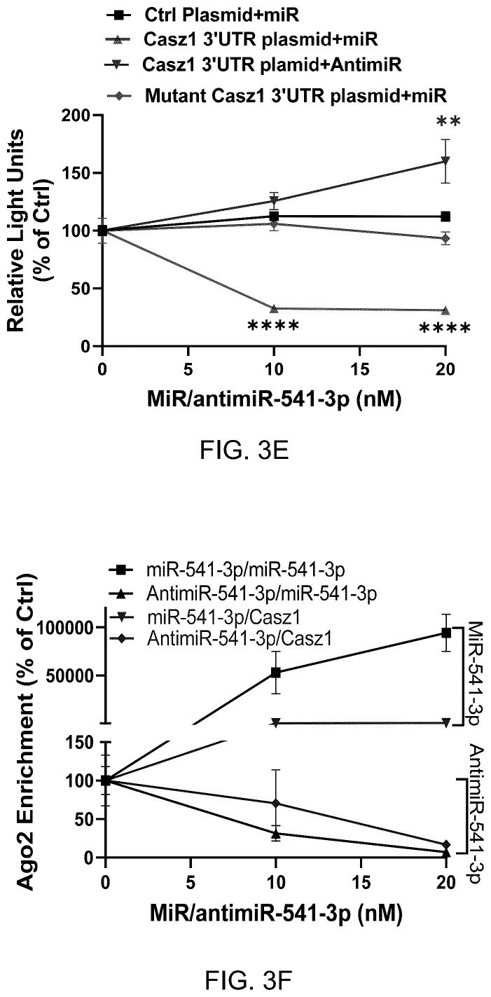

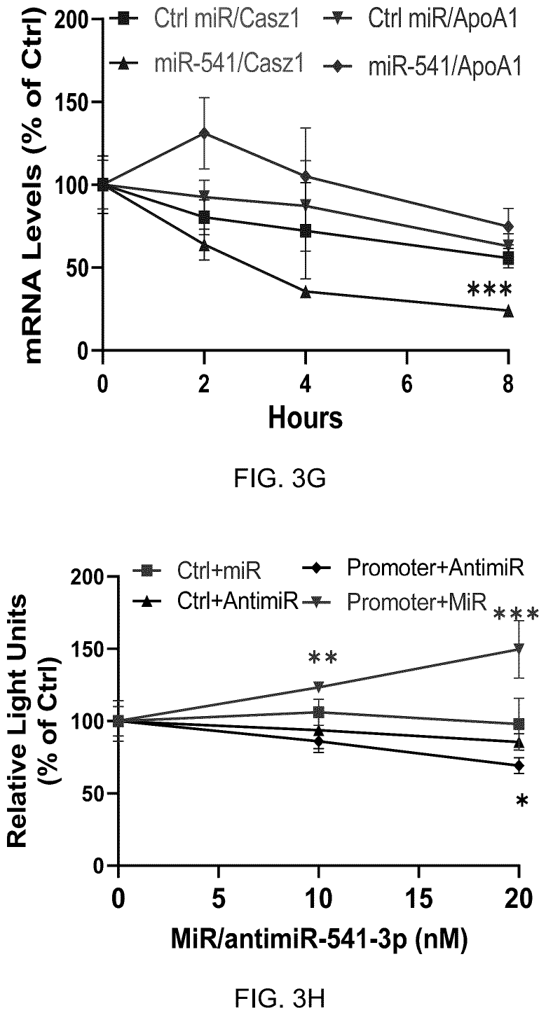

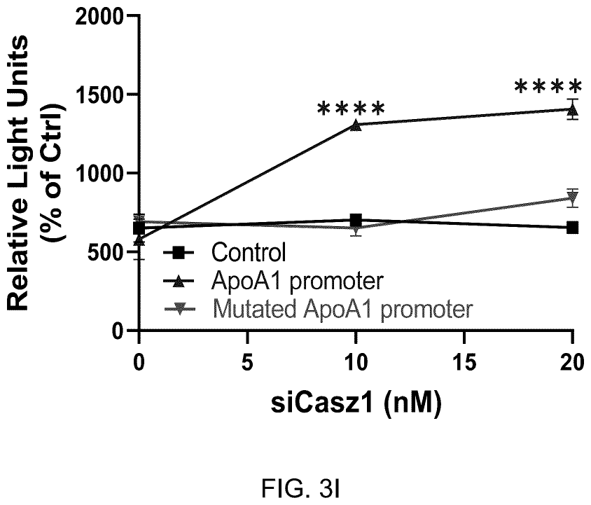

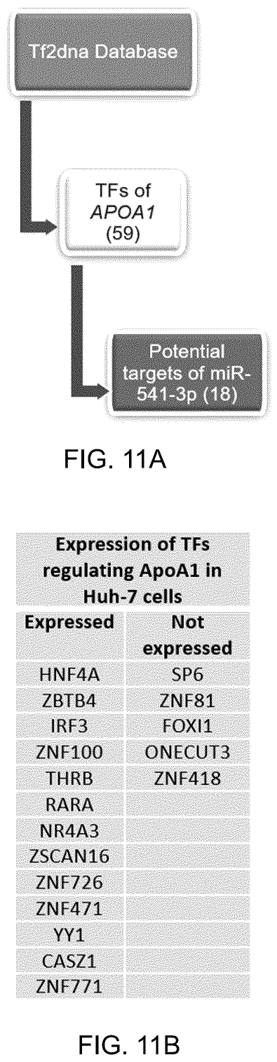

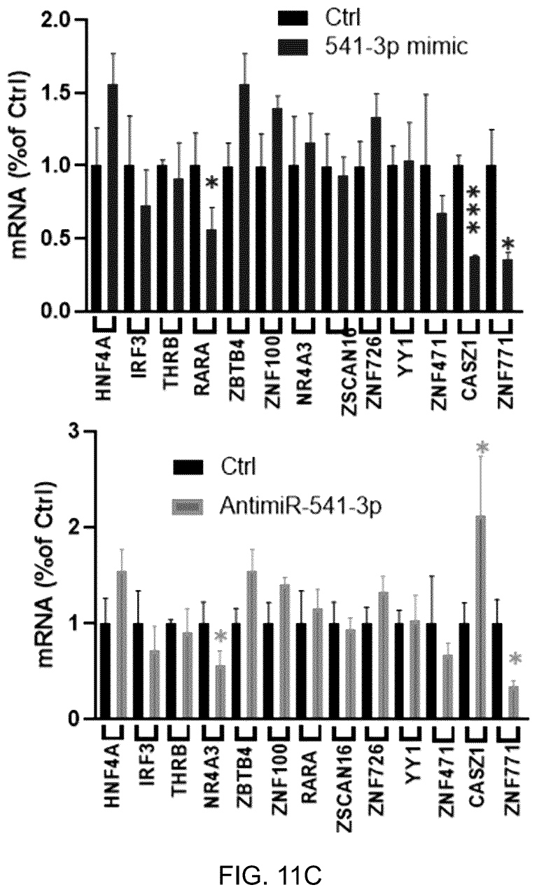

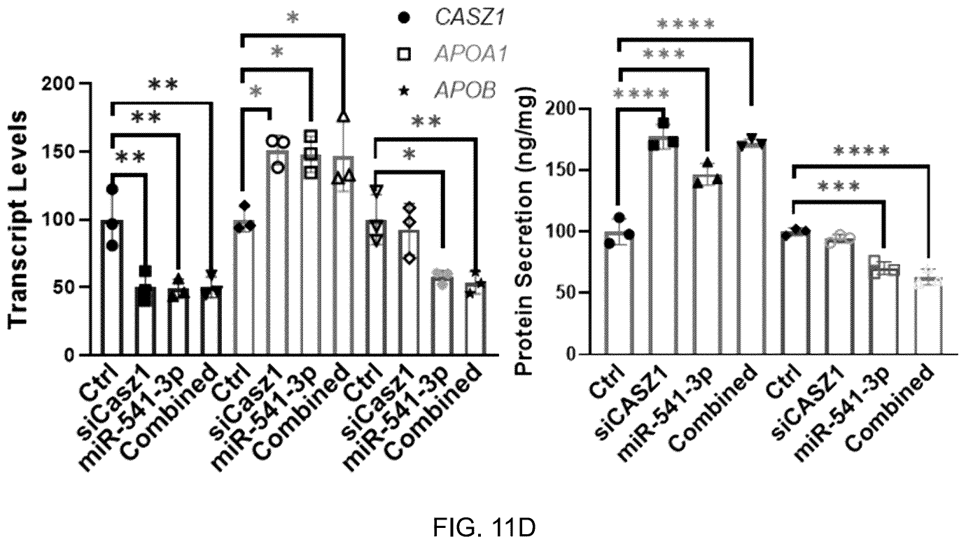

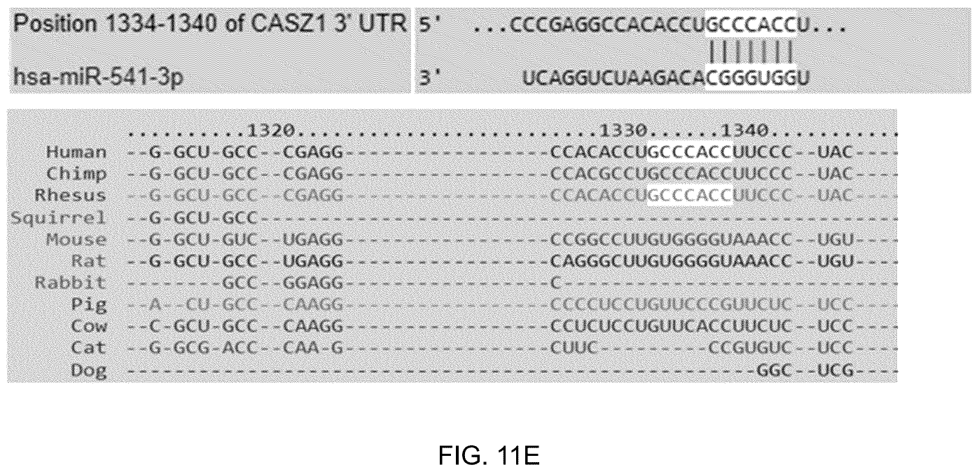

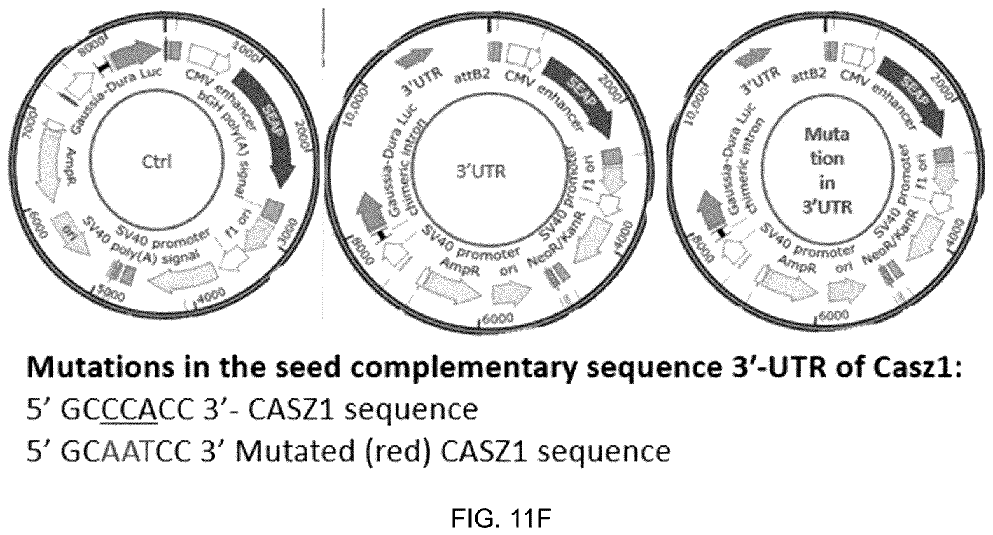

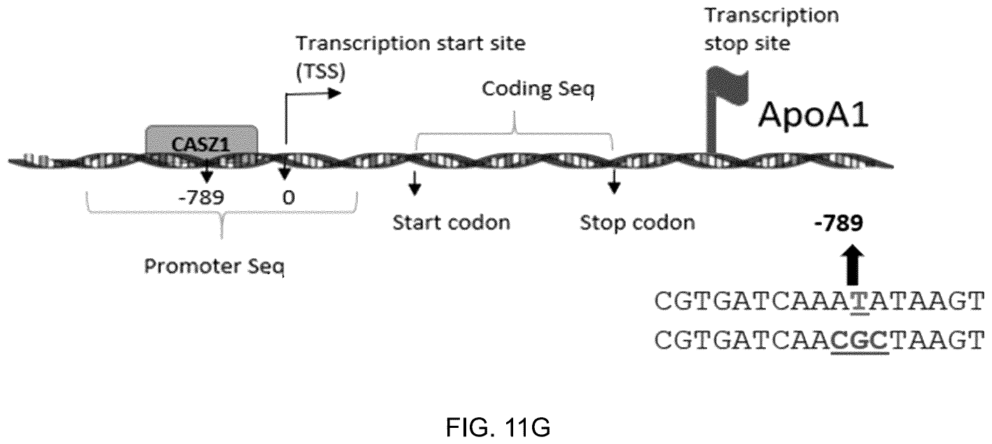

To understand regulation of apoA1 by miR-541-3p, we used a similar approach outlined above for apoB. Transfection of increasing concentrations of miR-541-3p mimics enhanced, while antimiR-541-3p decreased, APOA1 mRNA levels, respectively (FIG. 3A). APOA1 mRNA is not a target of miR-541-3p and BLAST revealed no complementarity between miR-541-3p and APOA1 mRNA and promoter. Thus, Watson-Crick base pairing between miR-541-3p and APOA1 mRNA is unlikely. Subsequently, we identified transcription factors that might regulate APOA1, contain miR-541-3p target sites in their 3′-UTRs, and are expressed in Huh-7 cells (FIG. 11A-B). Our studies found that transcript levels of CASZ1 were significantly decreased following overexpression of miR-541-3p mimics, while antimiR-541-3p had the opposite effect on CASZ1 mRNA (FIG. 3B, FIG. 11C). Transfection of siCASZ1 significantly reduced CASZ1 and increased APOA1 mRNA expression and protein secretion, without affecting apoB transcript or protein levels (FIG. 3C). Thus, miR-541-3p may reduce Casz1 to enhance apoA1 expression.

We then analyzed how miR-541-3p regulates Casz1. MiR-541-3p mimics and siCasz1, either individually or combined, decreased CASZ1 and increased APOA1 mRNA and protein secretion, to similar extents (FIG. 3D, FIG. 11D), indicating they are in the same pathway. TargetScan predicted that CASZ1 mRNA contains a miR-541-3p interacting site in its 3′-UTR that is conserved in primates (FIG. 11E). MiR-541-3p mimics decreased luciferase activity when expressed with the 3′-UTR of CASZ1 mRNA, whereas antimiR-541-3p increased luciferase activity (FIG. 3E). These effects were abolished after mutagenesis of the 3′-UTR of CASZ1 mRNA (FIG. 3E, FIG. 11F). Higher miR-541-3p and CASZ1 mRNA were in Ago2 precipitates from miR-541-3p mimics transfected cells, whereas cells transfected with antimiR-541-3p had lower miR-541-3p and CASZ1 mRNA (FIG. 3F). CASZ1, but not APOA1, mRNA was degraded faster in miR-541-3p mimic expressing cells compared to control cells (FIG. 3G). These studies indicated that miR-541-3p likely interacts with the 3′-UTR in Ago2 complexes and induces post-transcriptional degradation of CASZ1 mRNA.

We then investigated how Casz1 increases apoA1 expression. A potential Casz1 binding site is located at −789 bp of the transcription start site in the apoA1 promoter (FIG. 11G). MiR-541-3p mimics increased, while antimiR-541-3p decreased, luciferase activity under the control of the APOA1 promoter (FIG. 3H). Furthermore, siCASZ1 significantly increased APOA1 promoter activity, and this increased activity was abolished following mutagenesis of the Casz1 binding site in the APOA1-promoter (FIG. 3I). These studies indicate that Casz1 is a repressor of apoA1 expression.

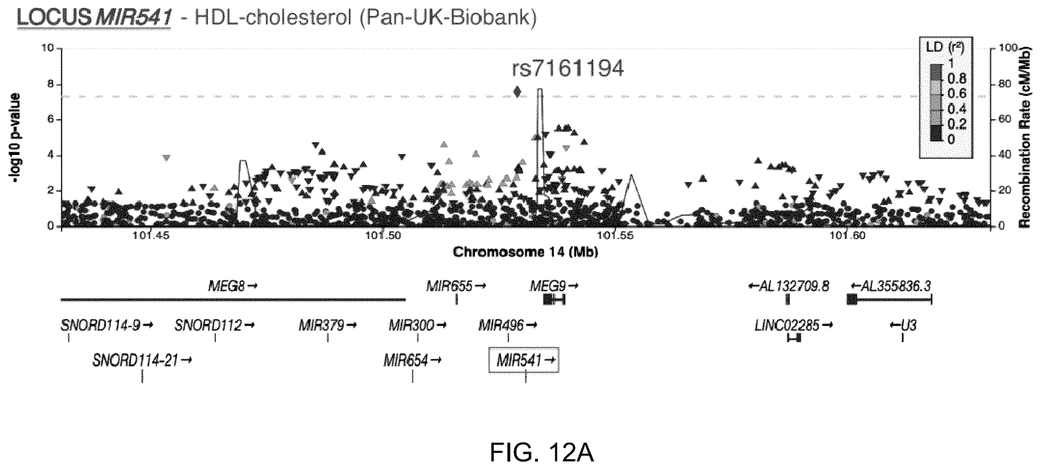

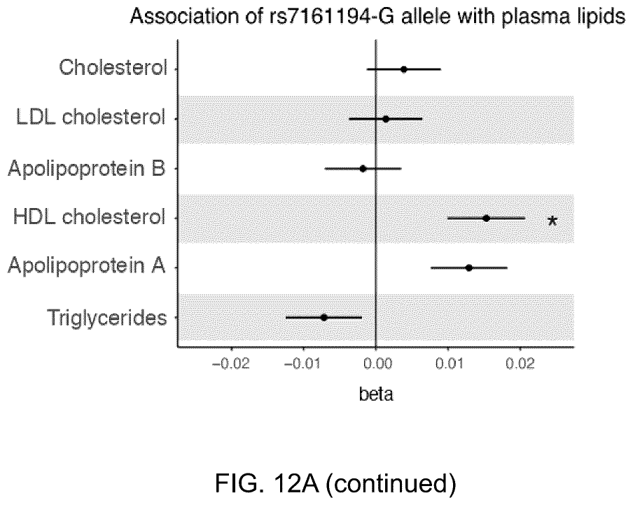

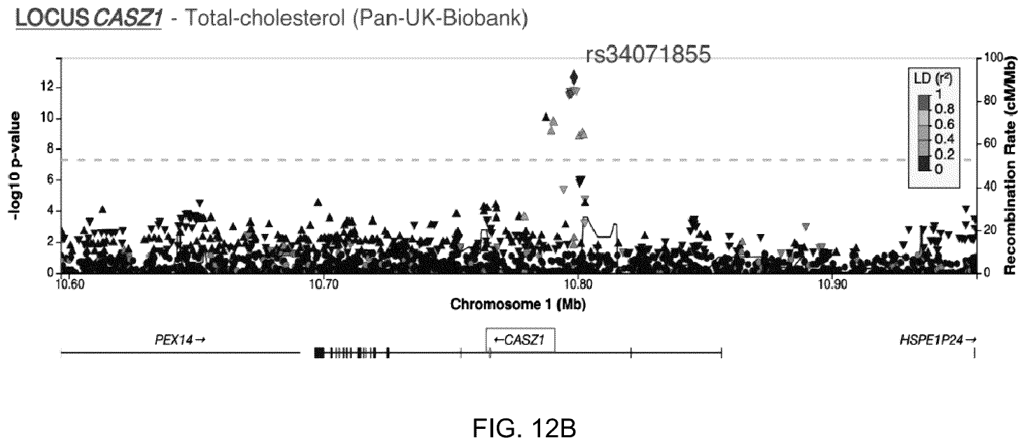

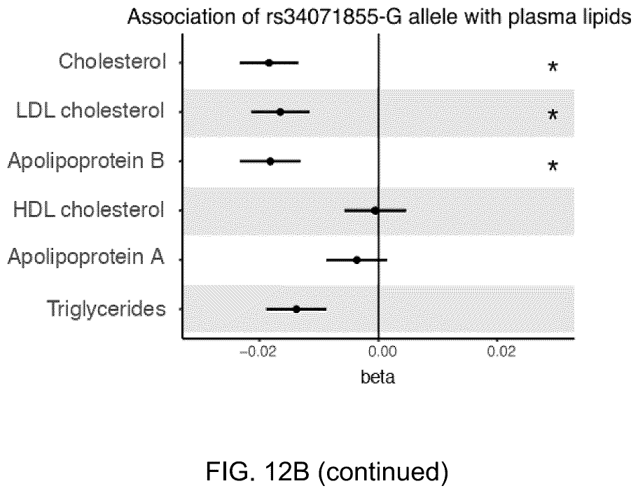

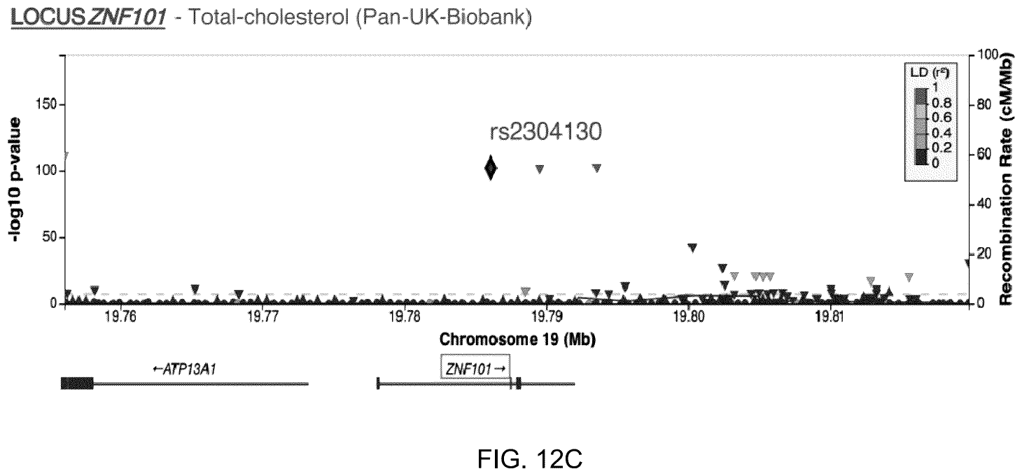

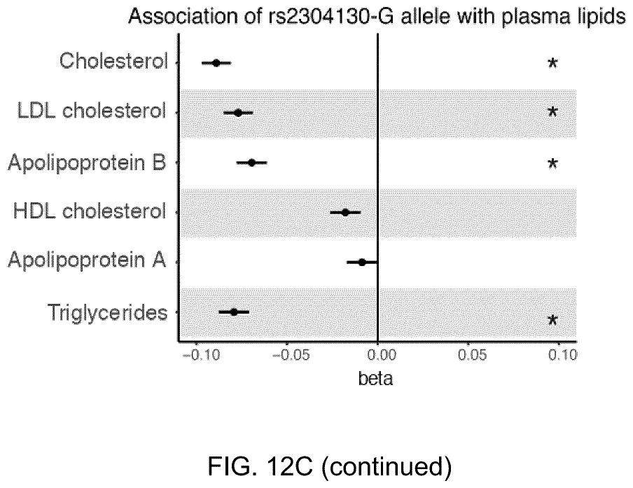

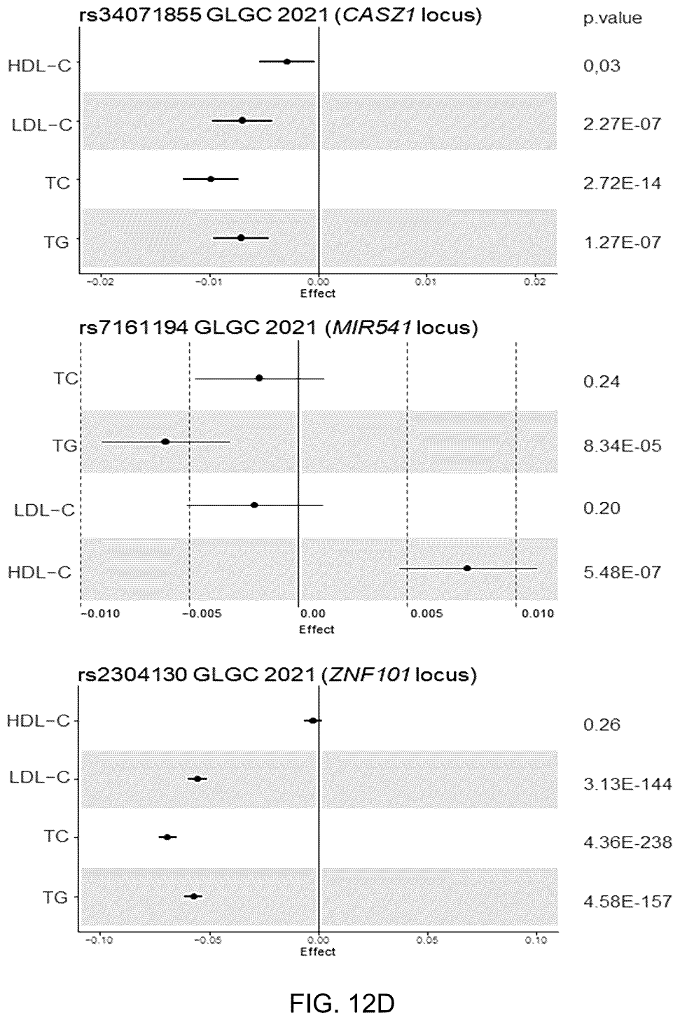

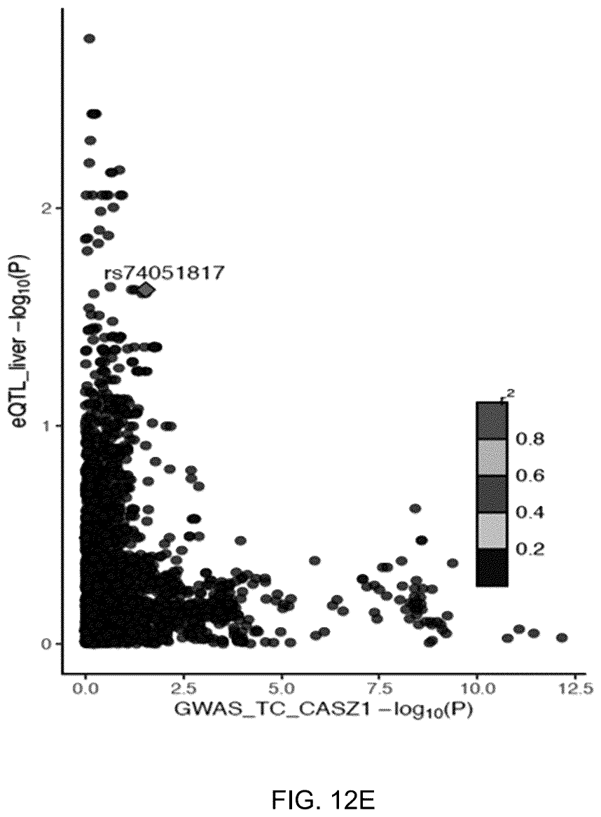

The above studies identified M/R541, ZNF101 and CASZ1 genes as modulators of apoB and apoA1 in human liver cells. To extend the relevance of these genes to humans, we screened for genetic associations of variants in these gene loci with plasma lipids in the UK-Biobank. We found significant associations between: rs7161194, located ˜1.8 kb upstream of M/R541, and higher plasma HDL-C levels; rs34071855, located in the second intron of CASZ1, and reduced plasma cholesterol, LDL-C and apoB levels; and rs2304130, located in the second intron of ZNF101, and reduced plasma cholesterol, LDL-C, apoB and triglyceride levels (FIG. 12A-C). These associations were further replicated in the Global Lipid Genetic Consortium (GLGC) dataset (16) (FIG. 12D-E). The observed significant associations suggest for possible role of these genes in human plasma lipid and lipoprotein levels.

### Example 4

### Mouse Orthologs of Human ZNF101 and CASZ1 Genes

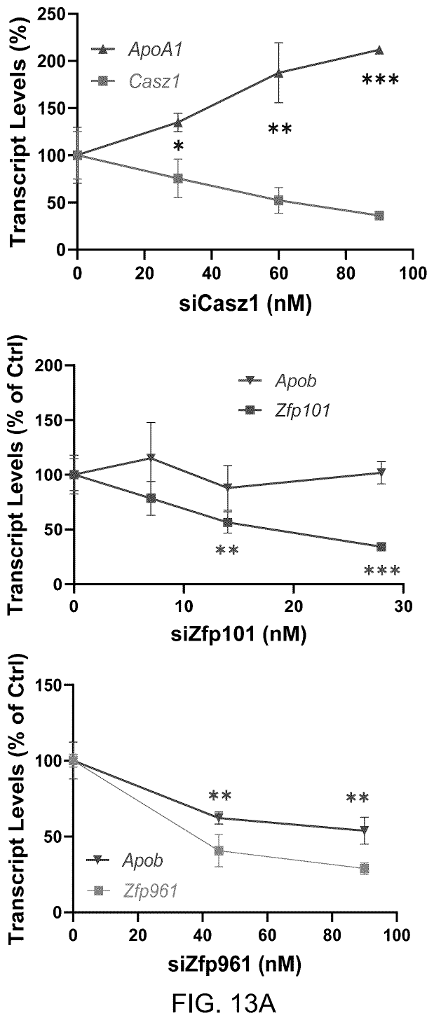

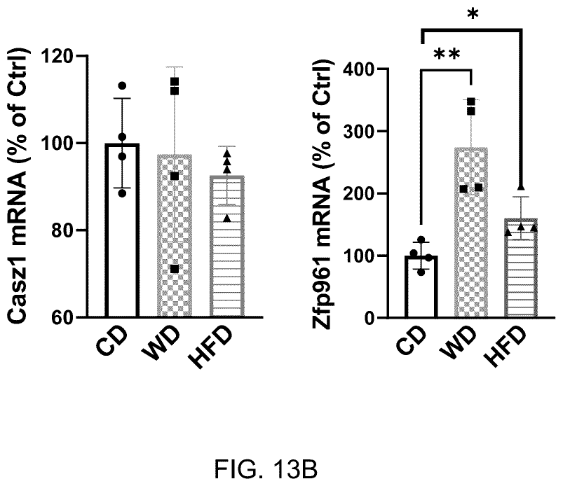

To evaluate in vivo roles of ZNF101 and CASZ1, we identified their mouse orthologs. Mouse Casz1 is an ortholog of human CASZ1 (GeneCards). Knockdown (KD) of Casz1 in mouse hepatocyte AML12 cells significantly increased Apoa1 expression (FIG. 13A). Mouse has two orthologs of human ZNF101, Zfp101 and Zfp961. The KD of Zfp101 had no effect on Apob mRNA. However, siZfp961 significantly reduced Zfp961 and Apob mRNA levels (FIG. 13A). Thus, mouse Casz1 and Zfp961 genes are functional orthologs of human CASZ1 and ZNF101 with respect to Apoa1 and Apob gene regulation. Since high fat diets affect plasma lipids and atherosclerosis, we next asked whether hepatic Casz1 and Zfp961 are regulated in vivo by these diets. C57Bl6 mice were fed chow, Western, or obesogenic diets. High fat diets increased Zfp961 mRNA levels but had no effect on Casz1 expression (FIG. 13B).

### Example 5

Hepatic Knockdowns of Casz1 and Zfp961 Alter Plasma apoB-Lps and HDL

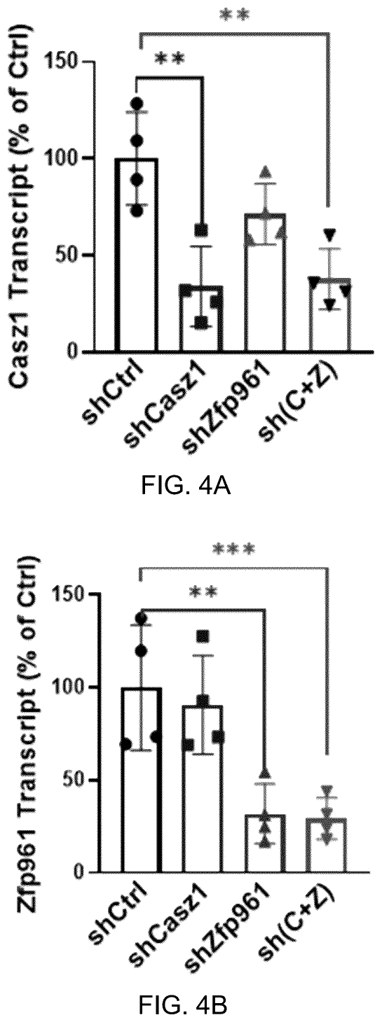

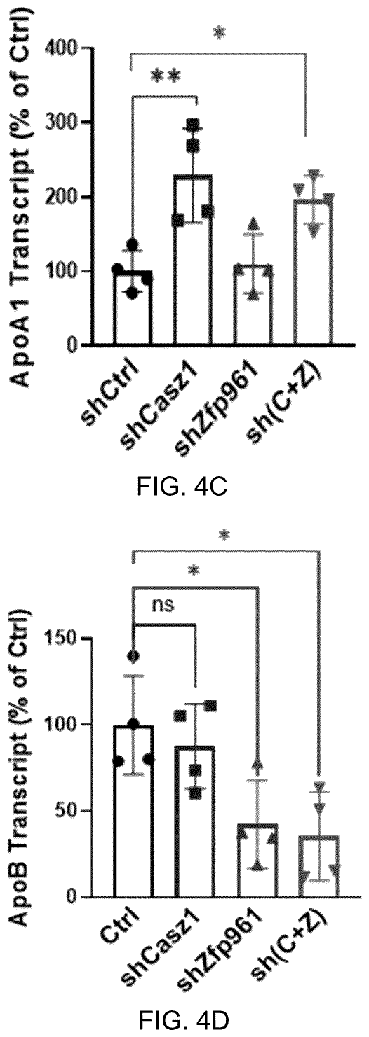

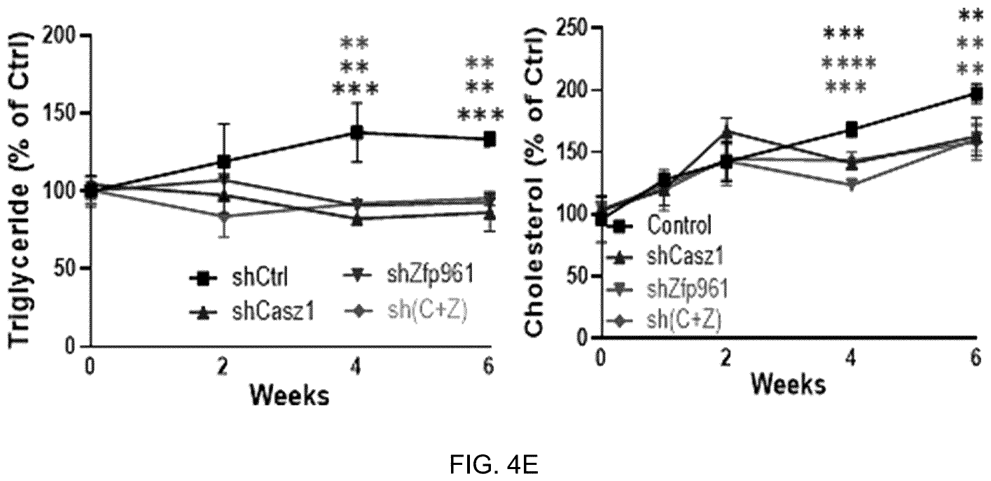

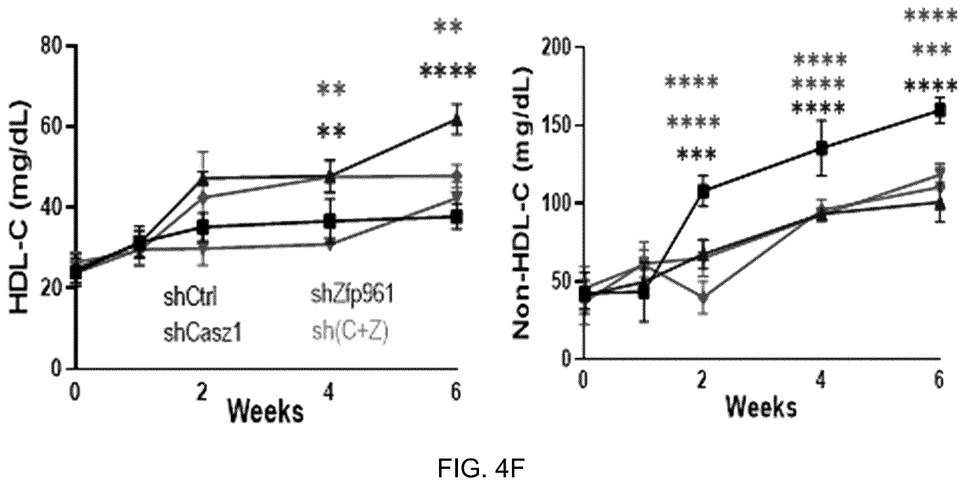

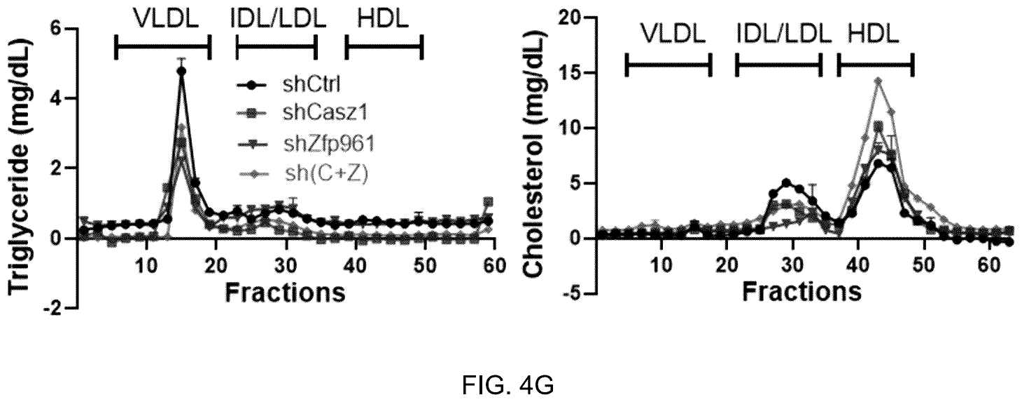

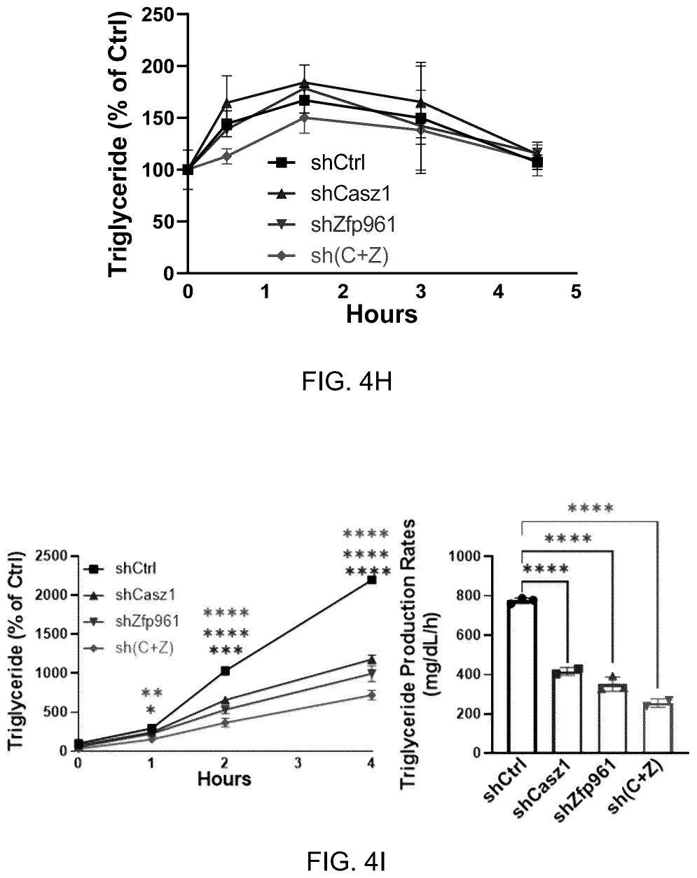

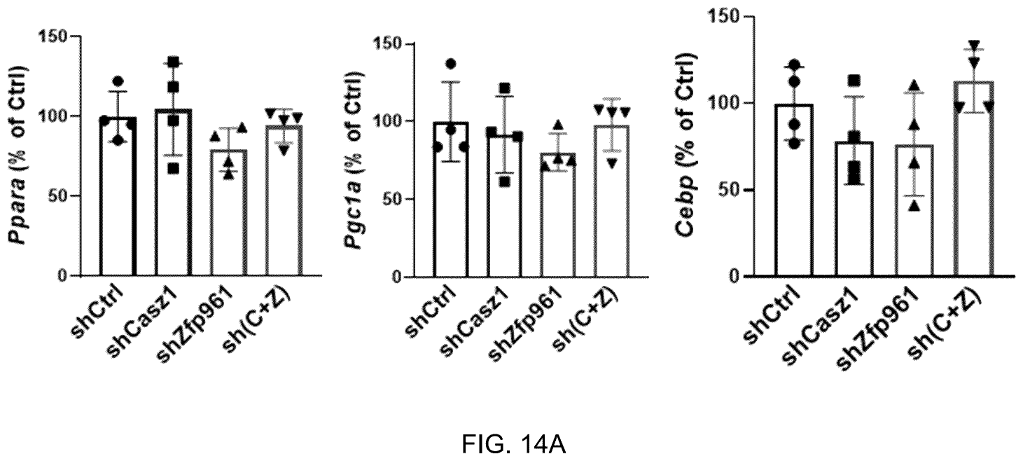

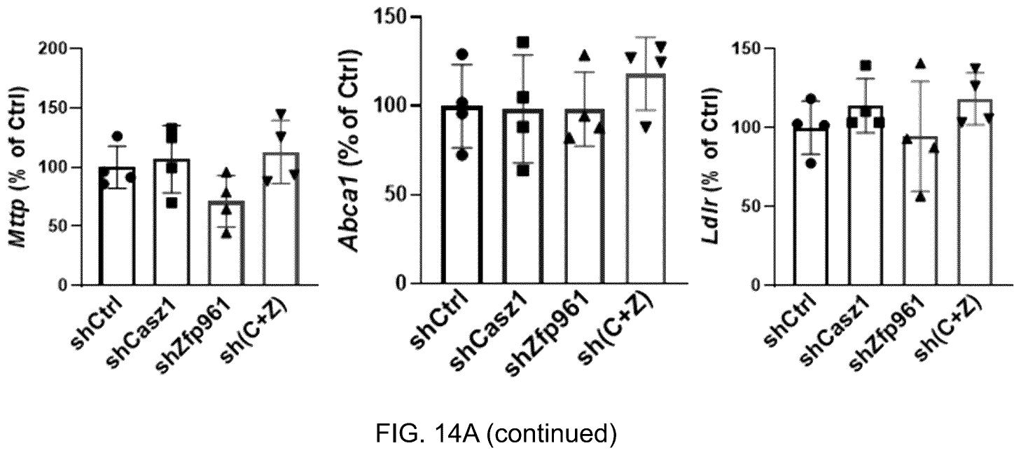

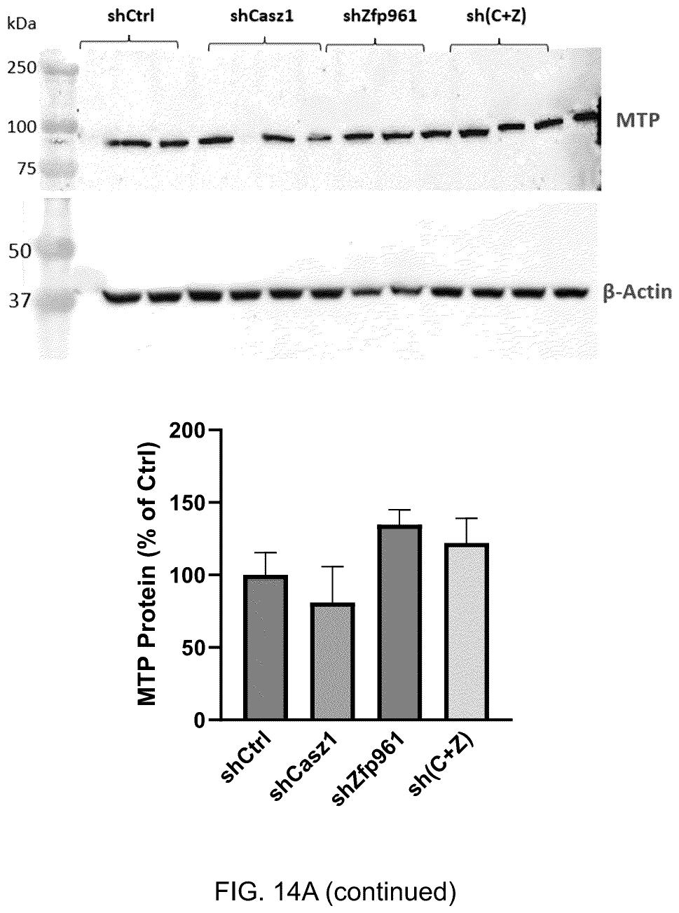

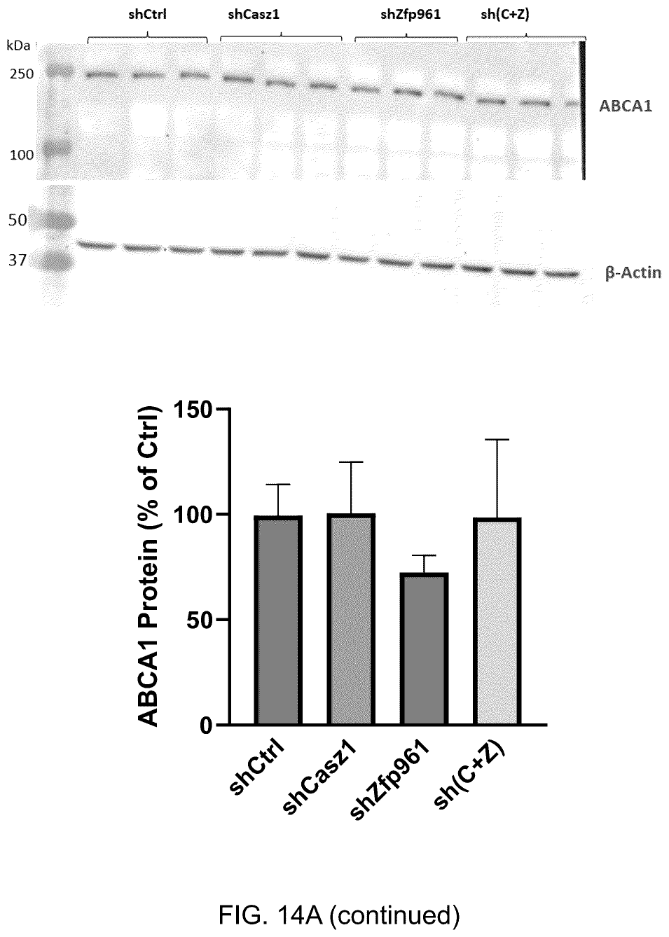

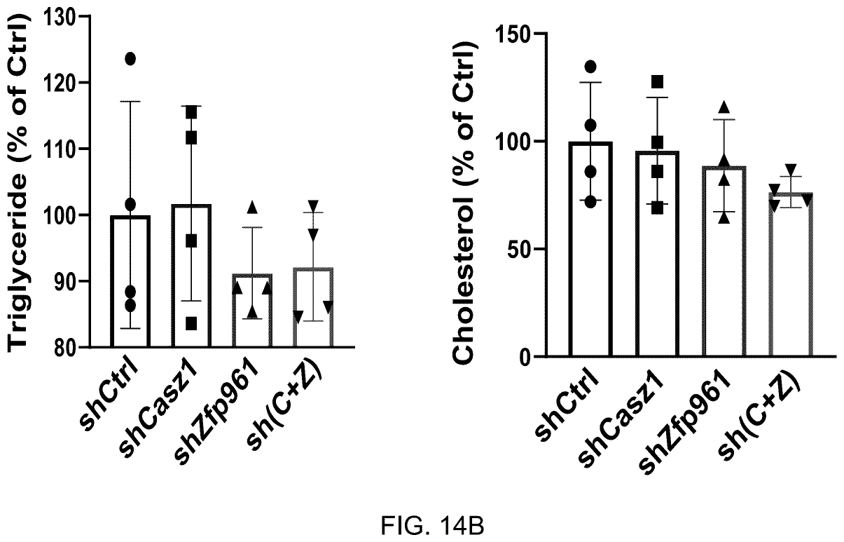

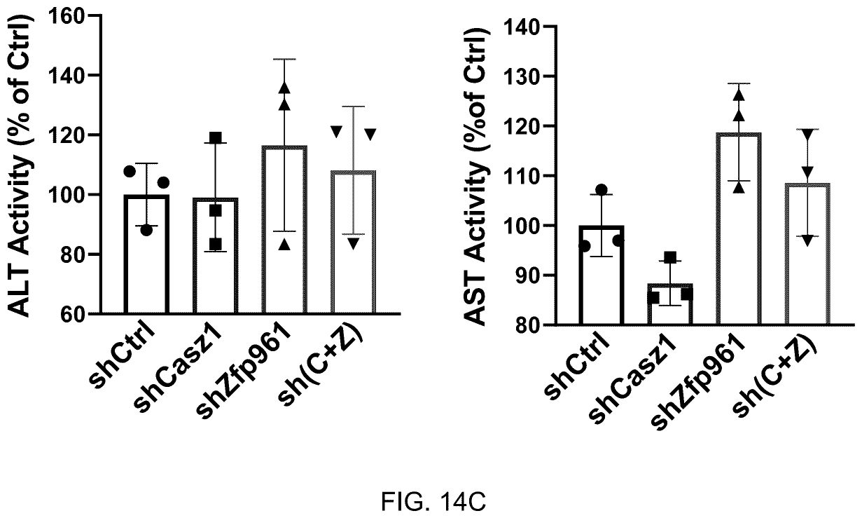

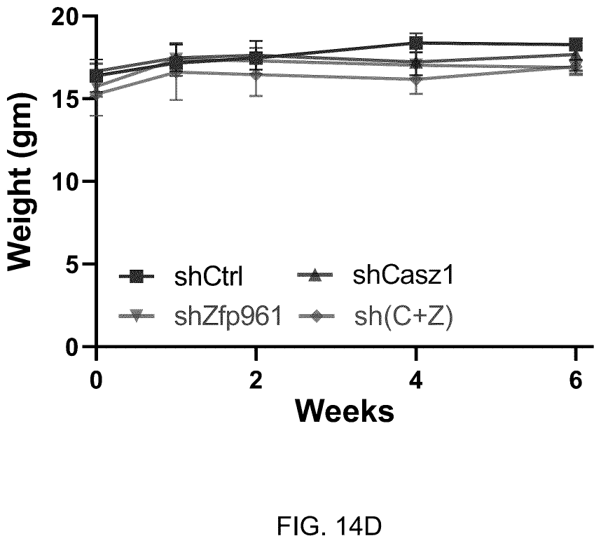

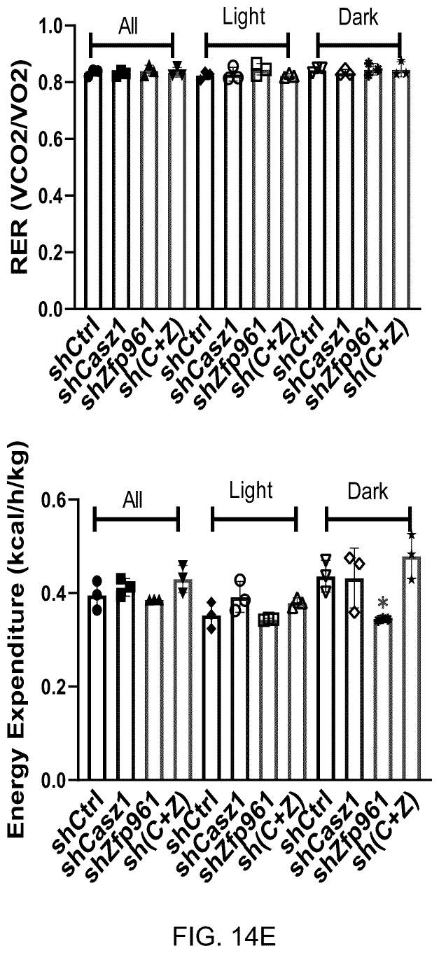

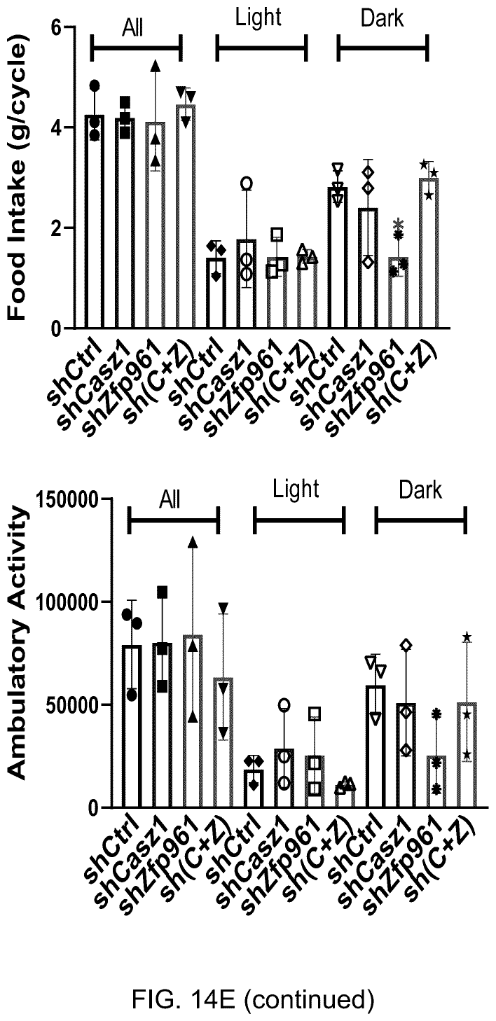

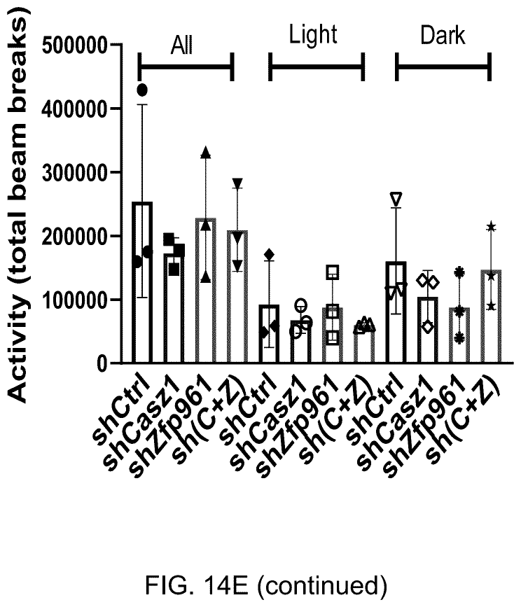

To test whether mouse Casz1 or Zfp961 regulate hepatic apoB and apoA1 levels, we transduced adult C57BL/6J mice with adenoviruses expressing shRNAs against both transcription factors, either alone or together, and fed them a high fat, high cholesterol Western diet. Both shCasz1 and shZfp961 specifically reduced hepatic Casz1 and Zfp961 mRNA, respectively, establishing their specificities (FIG. 4A-B). Hepatic Apoa1 mRNA levels increased (˜2.3-fold) in shCasz1 transduced mice, but remained undisturbed in shZfp961 transduced mice (FIG. 4C). Apob transcript levels did not change in shCasz1 transduced mice, but were significantly reduced (˜60%) in shZfp961 transduced mice (FIG. 4D). KD of Casz1 and Zfp961 had no effect on hepatic Ppara, Pgc1a, Cebp, Mttp, Abca1, and LdIrmRNA, hepatic triglyceride/cholesterol, and plasma ALT/AST levels compared to control mice (FIG. 14A-C). Similarly, these KDs had no effect on total weight gain, or on other physical and physiological parameters, but they reduced fat mass (FIG. 14D-E, Supplementary Table S2). Thus, reduction of hepatic Casz1 increases Apoa1, whereas Zfp961 KD decreases Apob expression.

We also studied the effects of their hepatic KDs on plasma lipids and lipoproteins. The rise in plasma triglyceride and cholesterol over time was significantly lower in shCasz1 and shZfp961 transduced mice compared to in shCtrl mice (FIG. 4E). HDL-cholesterol (HDL-C) significantly increased (˜63%) in shCasz1 and double shCasz1+shZfp961 transduced mice, but were unaffected by shZfp961 KD alone (FIG. 4F). KD of either Zfp961 and Casz1, or both together, however, significantly reduced non-HDL-C(FIG. 4F). Most of the triglycerides were in very low-density lipoprotein (VLDL) fractions in control mice, but the levels were reduced in all KD mice compared to controls (FIG. 4G). FPLC studies further showed that cholesterol was mainly in HDL of control mice. HDL-C was unaffected by shZfp961 but increased in mice transduced with shCasz1 and shCasz1+shZfp961 (FIG. 4G), indicating that increases in plasma cholesterol in shCasz1 mice are due to increases in HDL-C. These studies showed that hepatic KDs of Casz1 and Zfp961, either separately or together, reduce triglyceride and non-HDL-C, whereas only Casz1 KD increases HDL-C. Importantly, KD of these transcription factors had no effect on intestinal lipid absorption (FIG. 4H); while hepatic triglyceride production rates were significantly reduced (FIG. 4I). The disclosure therefore shows that hepatic inhibition of both Casz1 and Zfp961 may reduce plasma lipids and apoB-Lps, as a result of impaired hepatic lipoprotein production.

### Example 6

### Atherosclerosis is Reduced Following Hepatic Knockdown of Casz1 and Zfp961

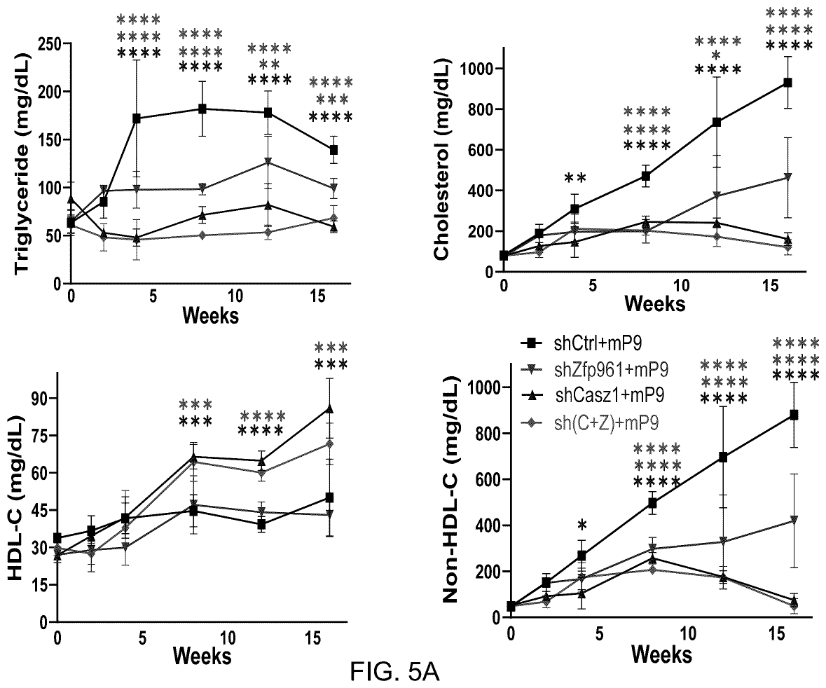

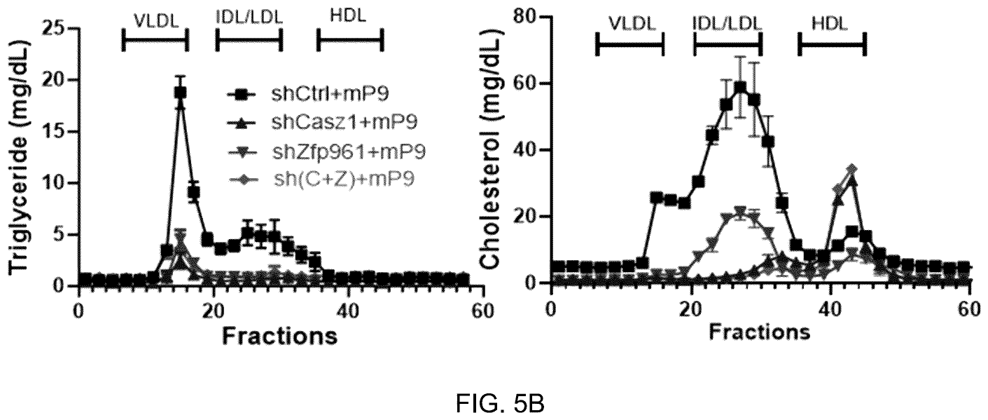

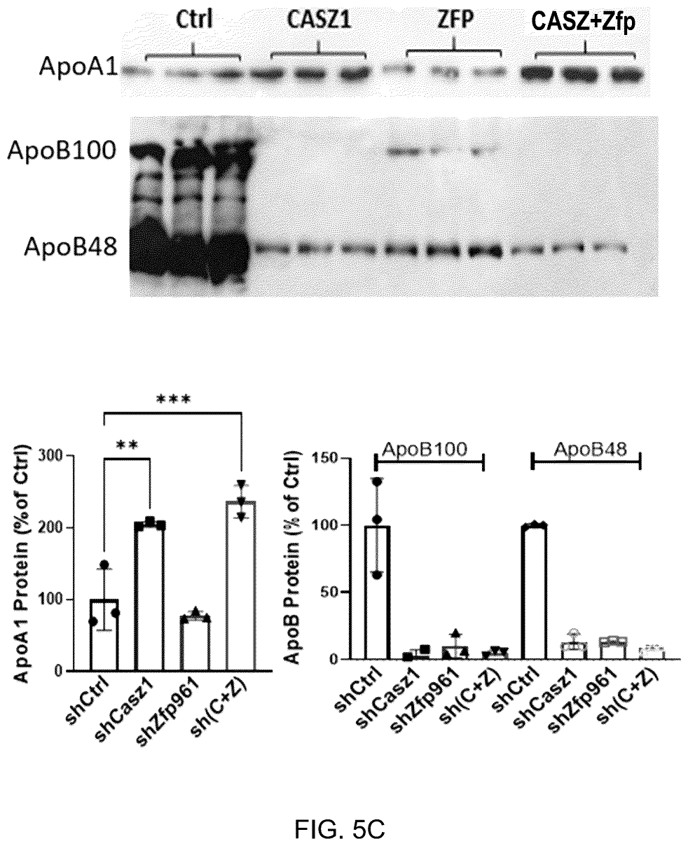

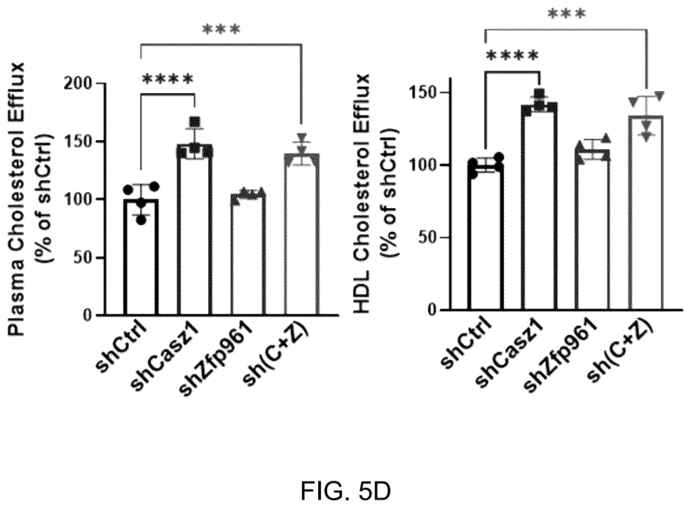

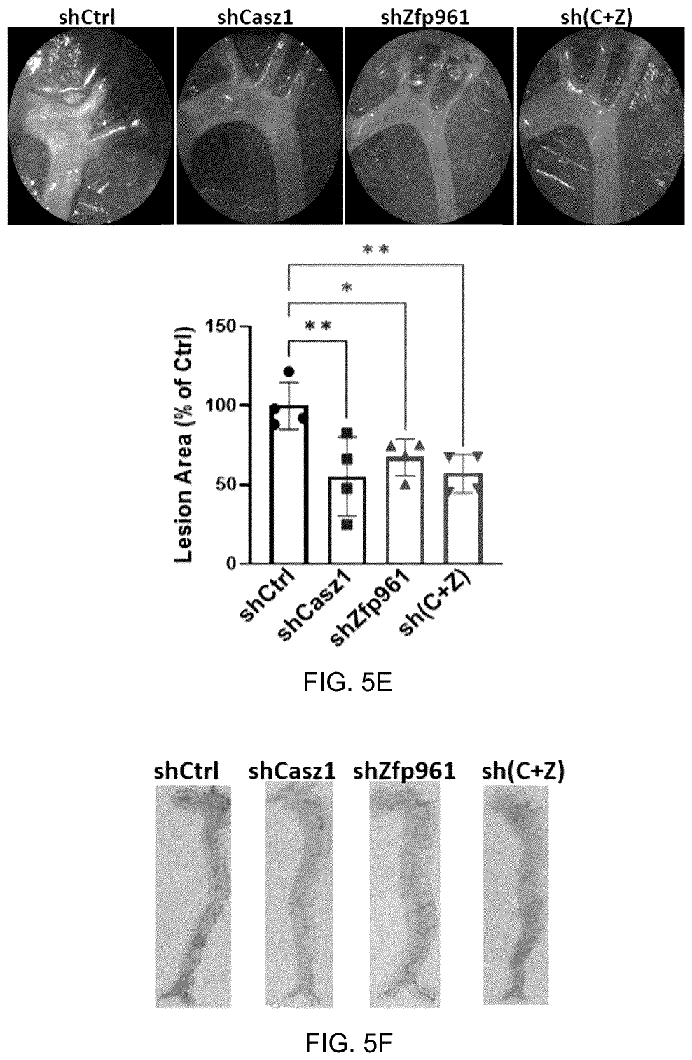

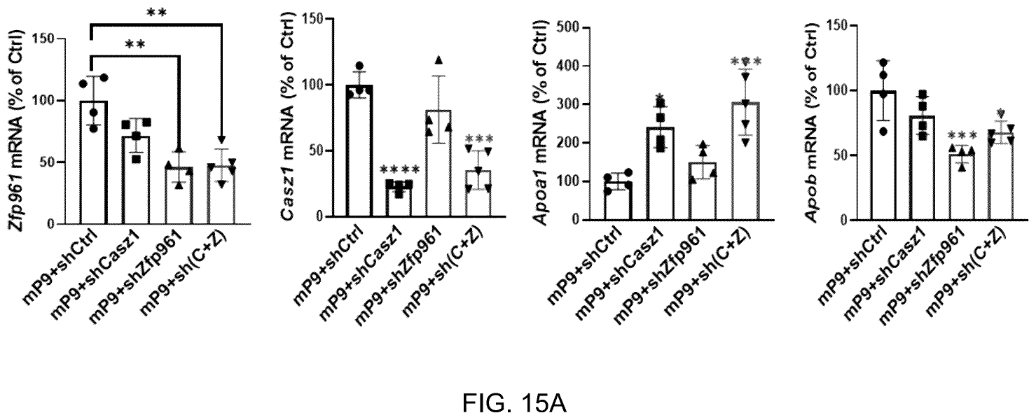

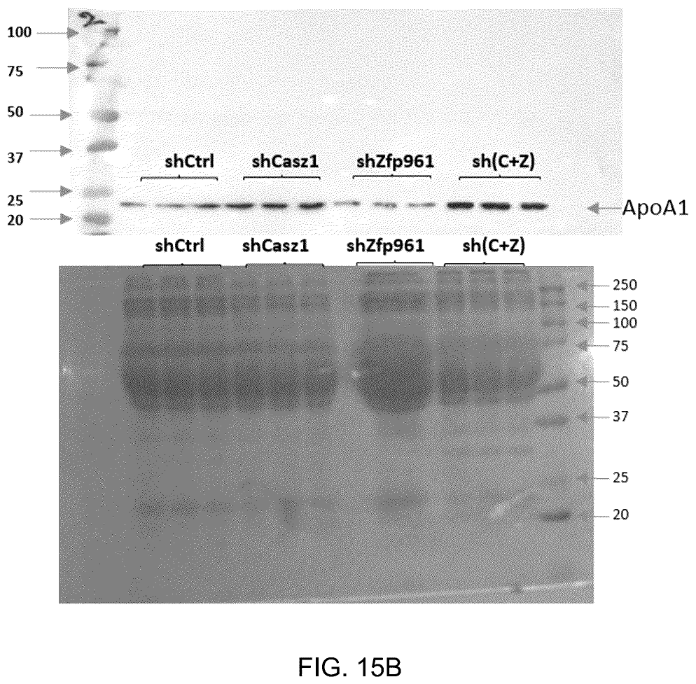

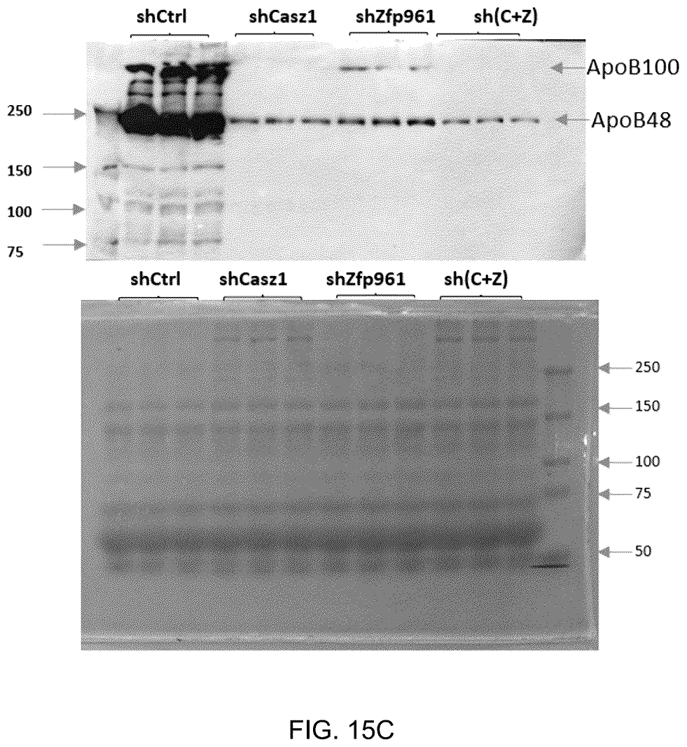

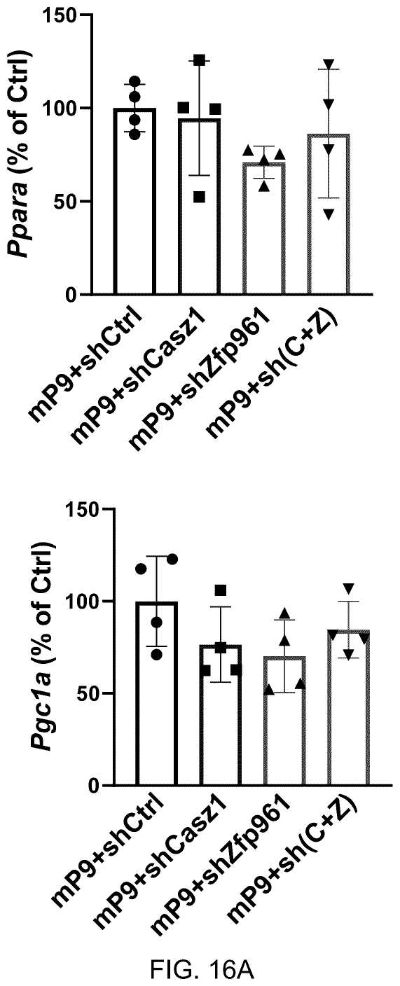

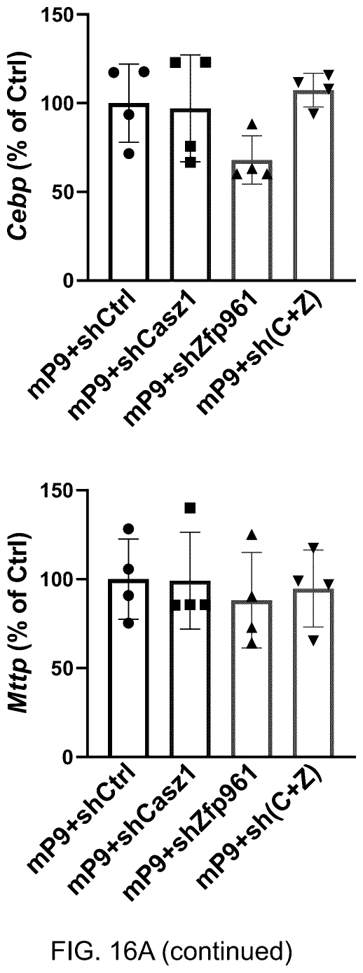

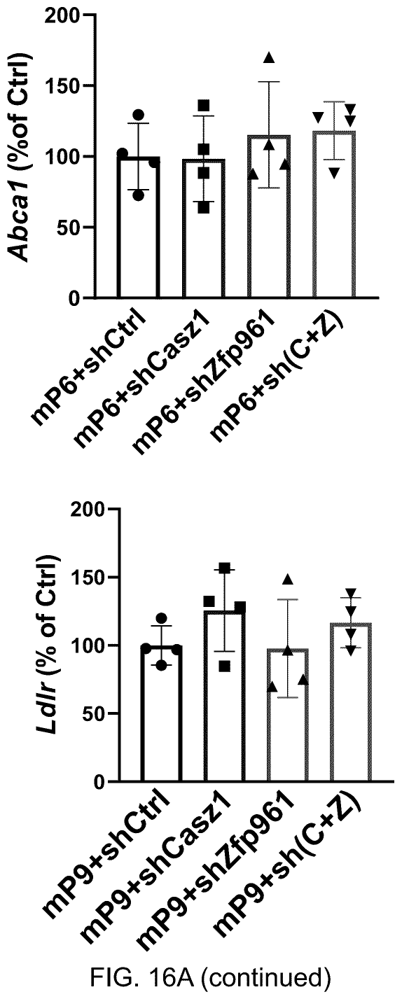

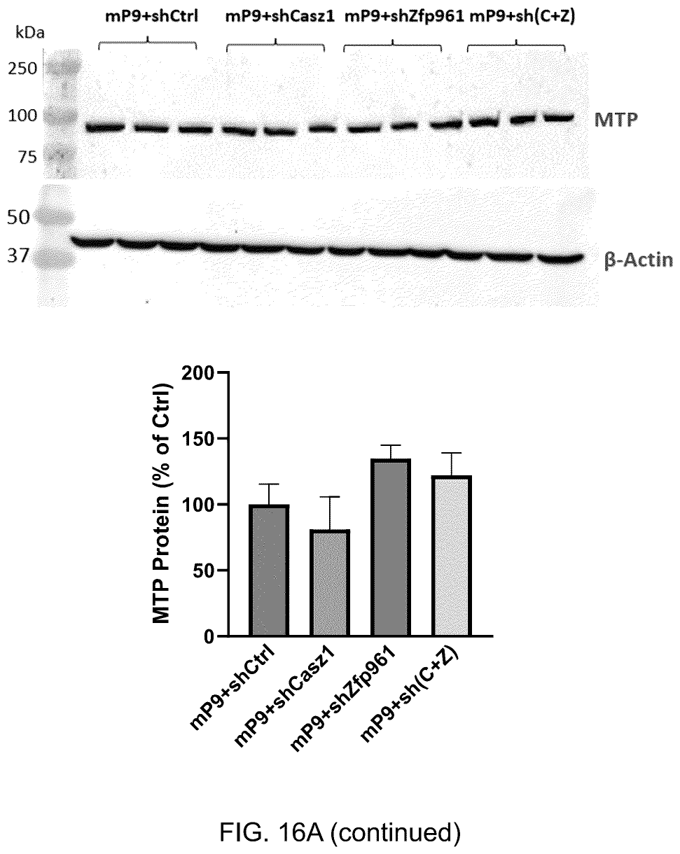

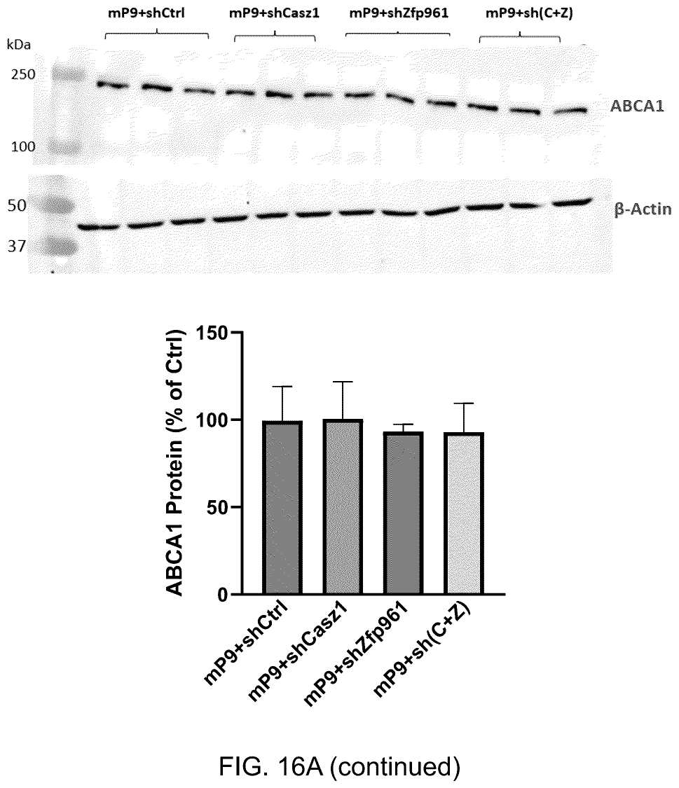

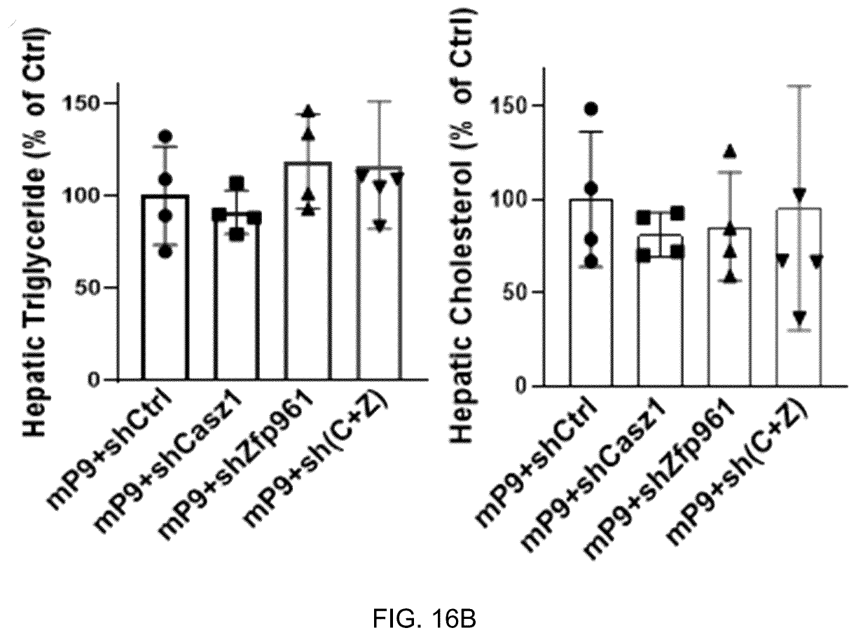

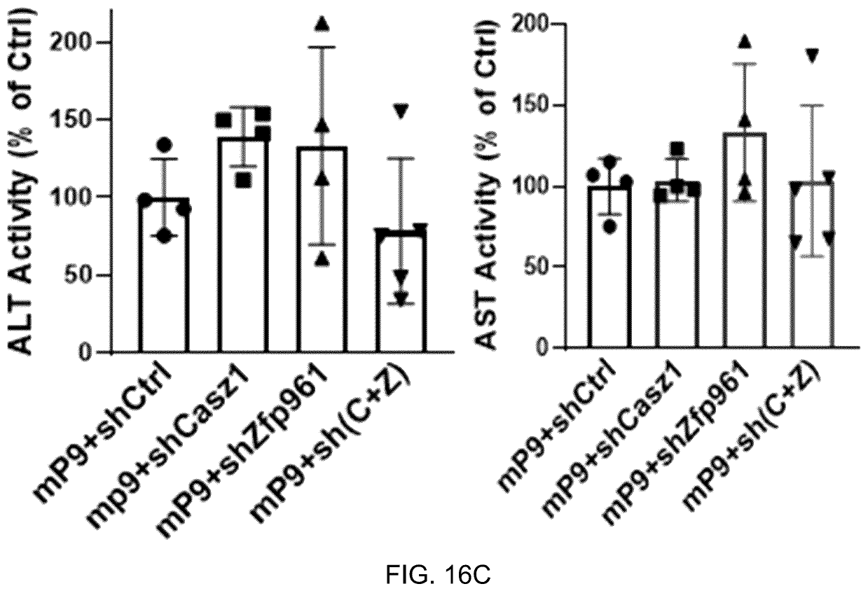

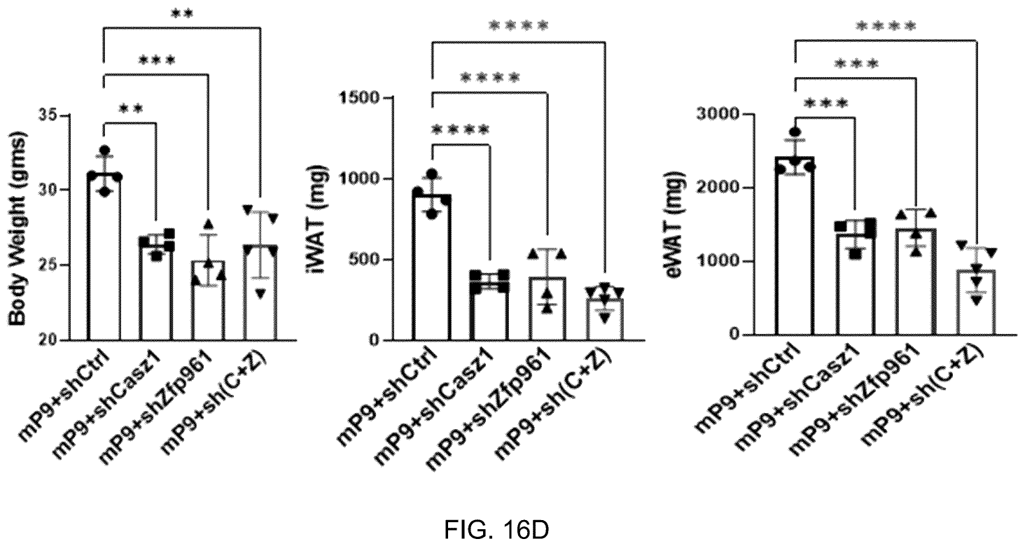

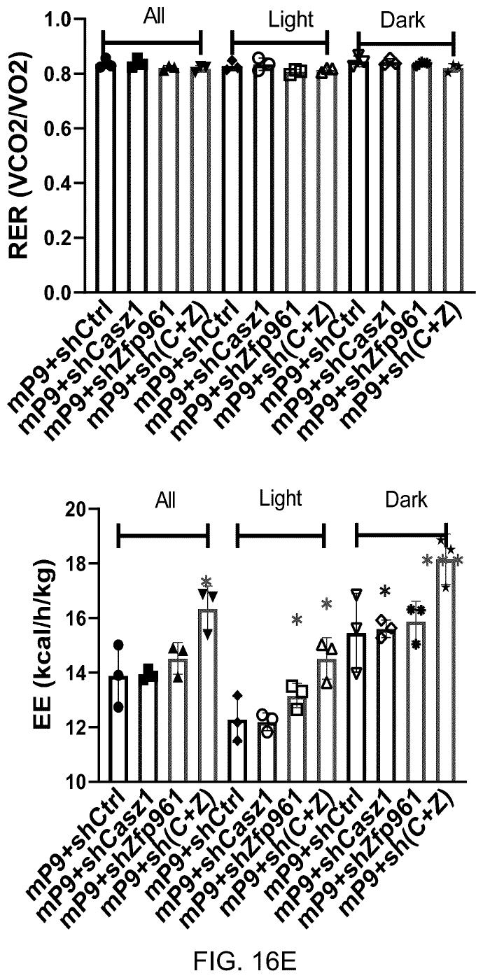

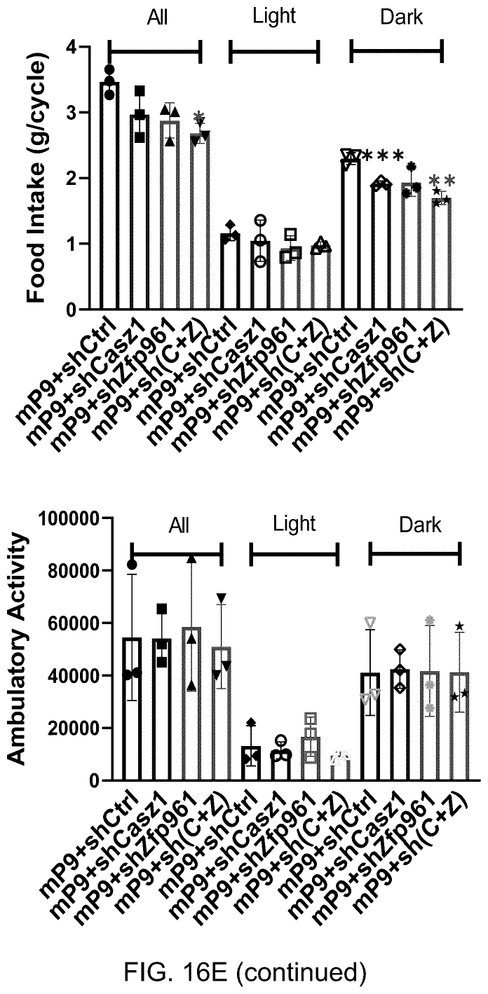

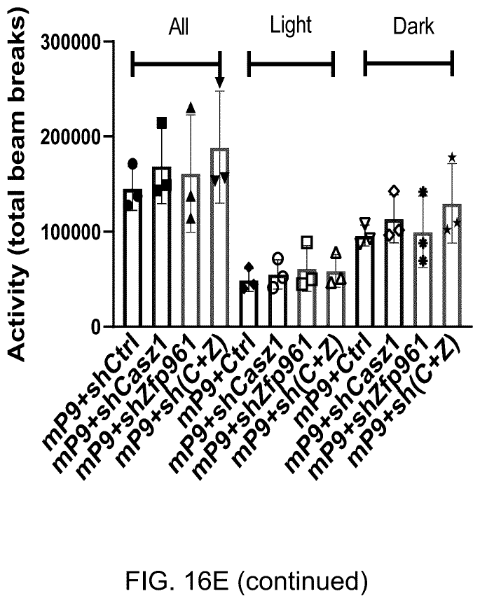

To test whether KD of Casz1 and/or Zfp961 could reduce atherosclerosis, we used a mouse atherosclerosis model, in which all adult mice were transduced with mutant mouse gain-of-function Pcsk9 (mPcsk9) (17). mPcsk9 augments degradation of hepatic LDL receptors, increases plasma cholesterol levels and induces atherosclerosis. ShZfp961 and shCasz1 both specifically reduced their own target mRNAs; while shZfp961 reduced apoB mRNA levels without affecting apoA1, shCasz1 increased apoA1 without affecting apoB mRNA levels (FIG. 15A). Control mice transduced with mPcsk9+shCtrl showed increased plasma triglyceride, cholesterol and non-HDL-C(FIG. 5A). Mice transduced with shCasz1 and shZfp961, on the other hand, had significantly reduced plasma levels of triglyceride, cholesterol, and non-HDL-C, while the shCasz1 transduced mice also had higher HDL-C levels compared to shCtrl (FIG. 5A). Triglyceride and cholesterol in VLDL and IDL/LDL fractions were significantly reduced in all KD mice compared to controls (FIG. 5B). In contrast, KD of Casz1 significantly increased plasma HDL-C levels. Plasma apoA1 protein levels increased in shCasz1 transduced mice but they were not affected by shZfp961 (FIG. 5C, FIG. 15B). ApoB100 and apoB48 levels were significantly reduced in all KD mice (FIG. 5C, FIG. 15C). ShCasz1 and shZfp961 had no significant effect on other TFs, lipoprotein genes, hepatic lipids and plasma transaminases (FIG. 16A-C). Thus, KDs of Casz1 and Zfp961 reduce plasma cholesterol and triglyceride in apoB-Lps without affecting hepatic lipids, whereas Casz1 KD, in addition, increases HDL-C.

We analyzed whether increased HDL in Casz1 KD mice is functional and tested its ability to efflux cholesterol from macrophages. Plasma and HDL from shCasz1 transduced mice showed increased cholesterol efflux from J774 macrophages compared to shCtrl (FIG. 5D) indicating increased cholesterol efflux capacity.

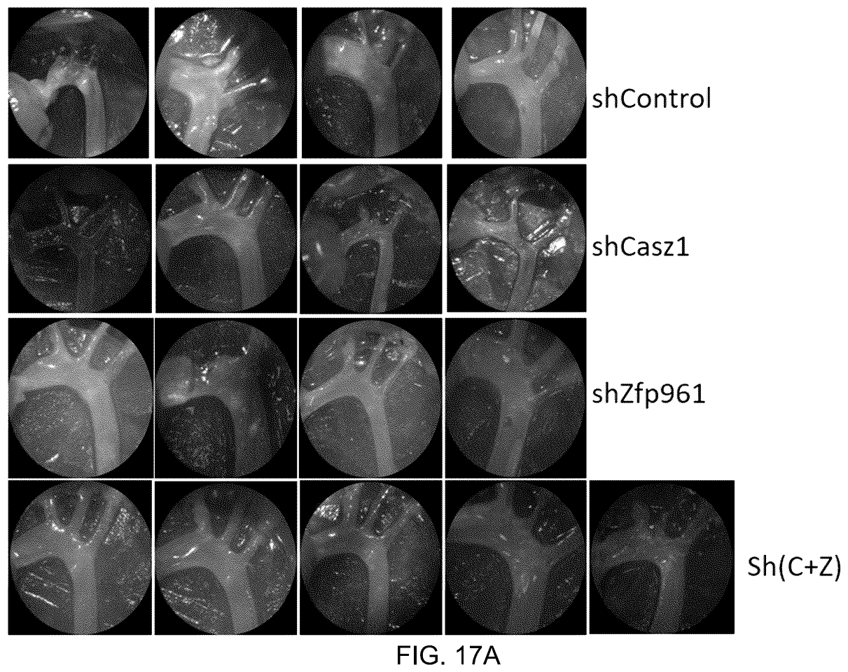

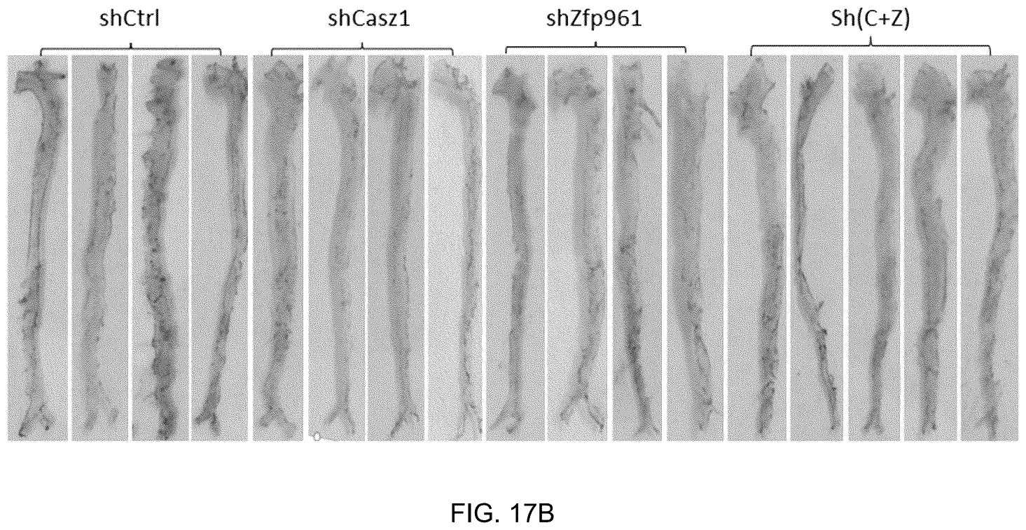

We then studied the effects of these KDs on physical and physiological parameters by DEXA and CLAMS, respectively. ShCasz1 and shZfp961 transduced mPcsk9 mice gained less weight and had significantly less fat than control mPcsk9 mice (FIG. 16D, Supplementary Table S3). ShCasz1 increased energy expenditure and reduced food intake in the dark, but shZfp961 had no effect (FIG. 16E). In addition, we visualized atherosclerotic plaques at the end. Atherosclerotic plaques were significantly reduced by ˜40-50% in all transcription factor KD mPcsk9 mice compared with controls (FIG. 5E-F, FIG. 17A-B). These studies show that KD of these TFs reduces atherosclerosis.

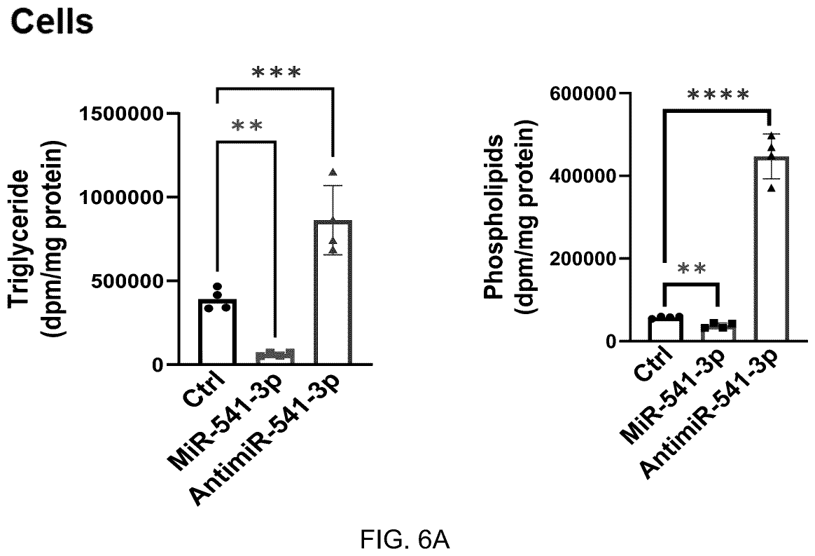

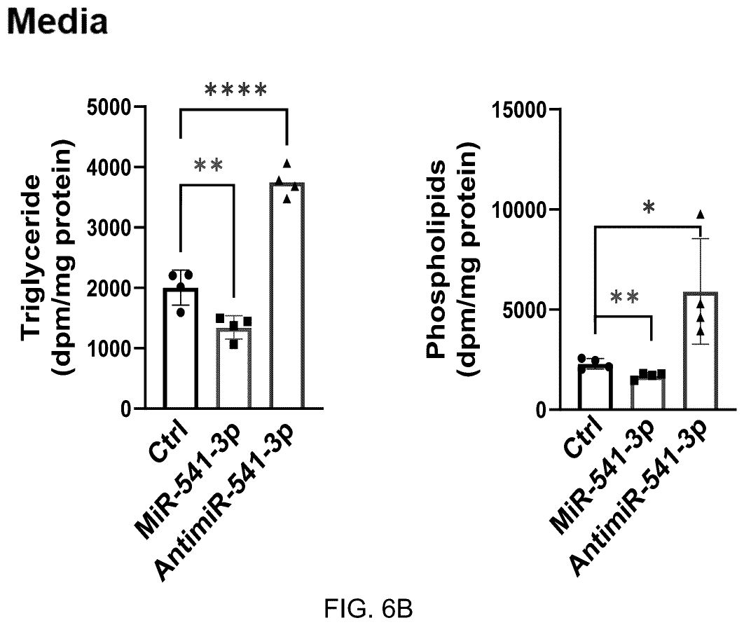

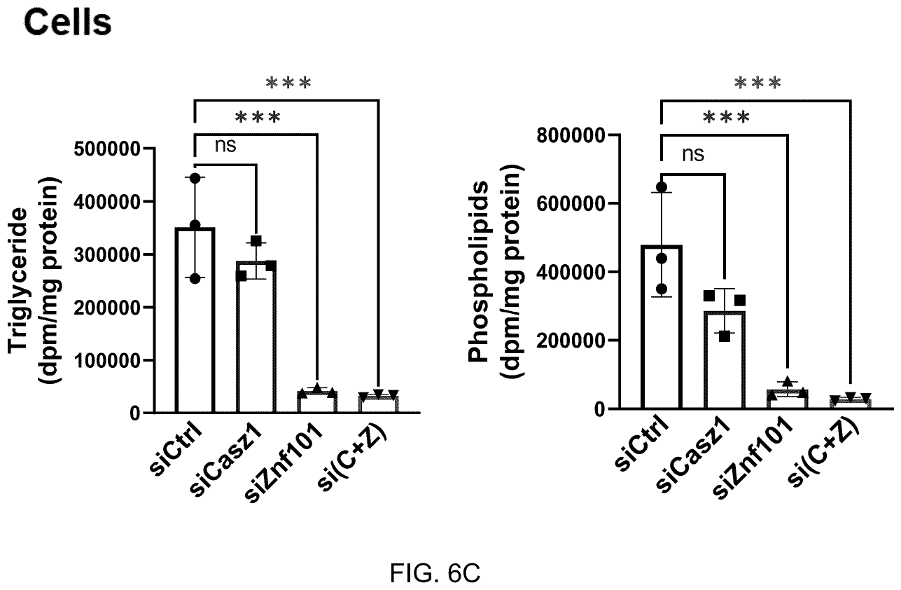

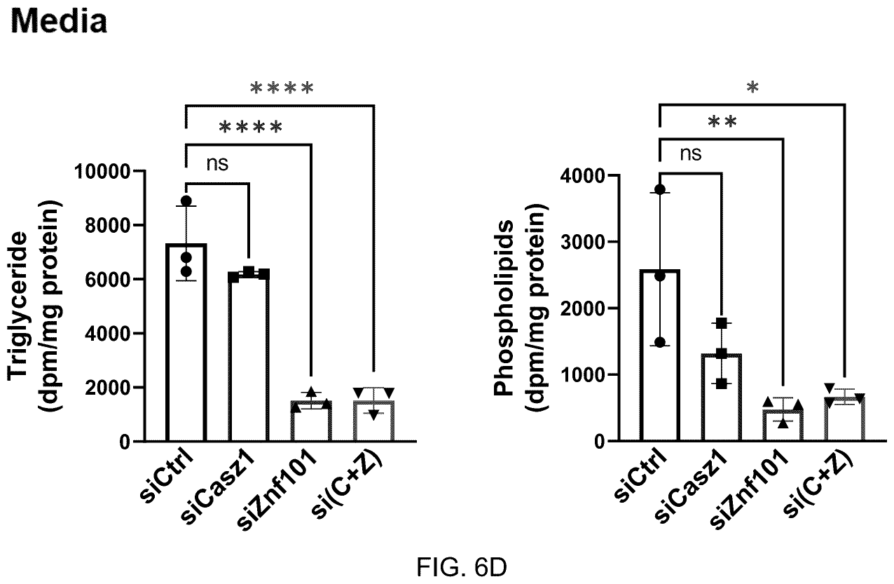

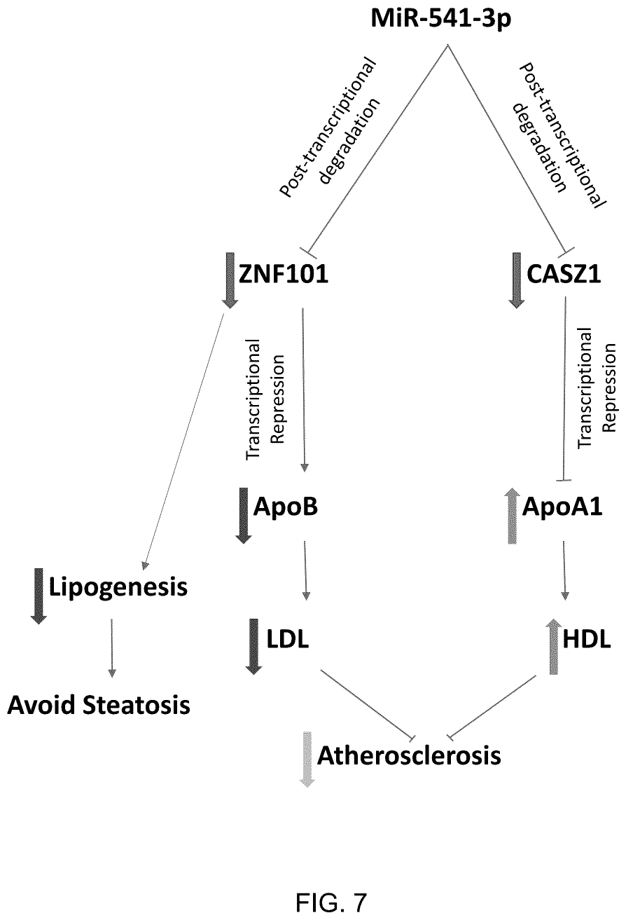

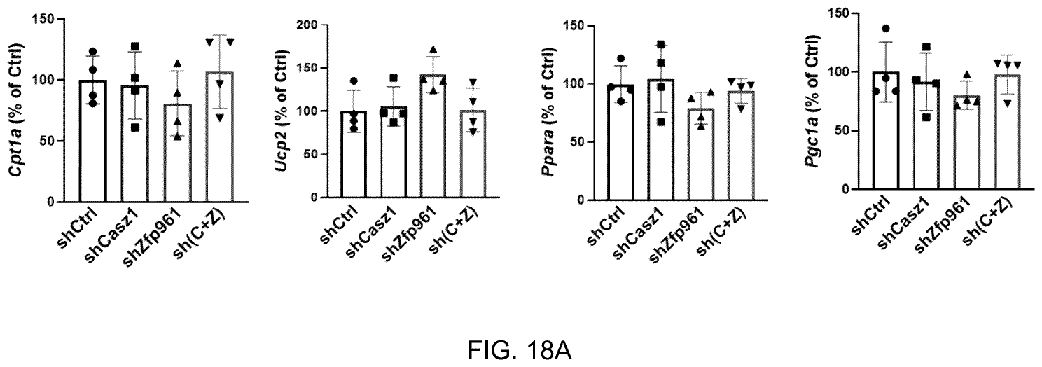

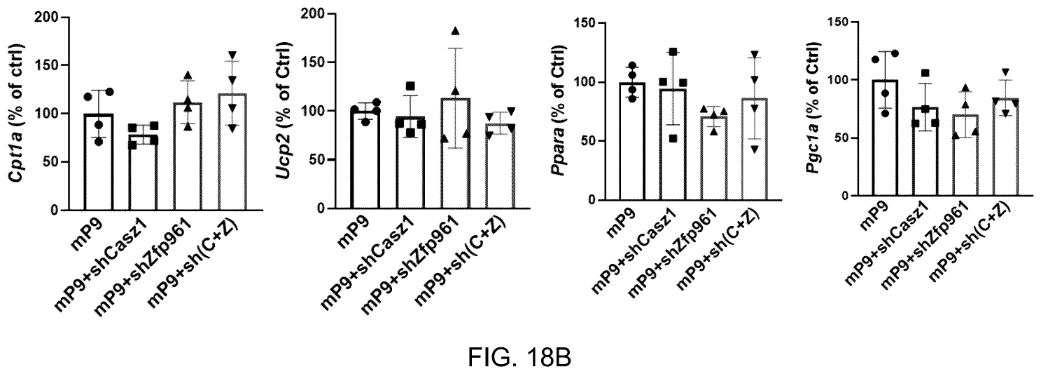

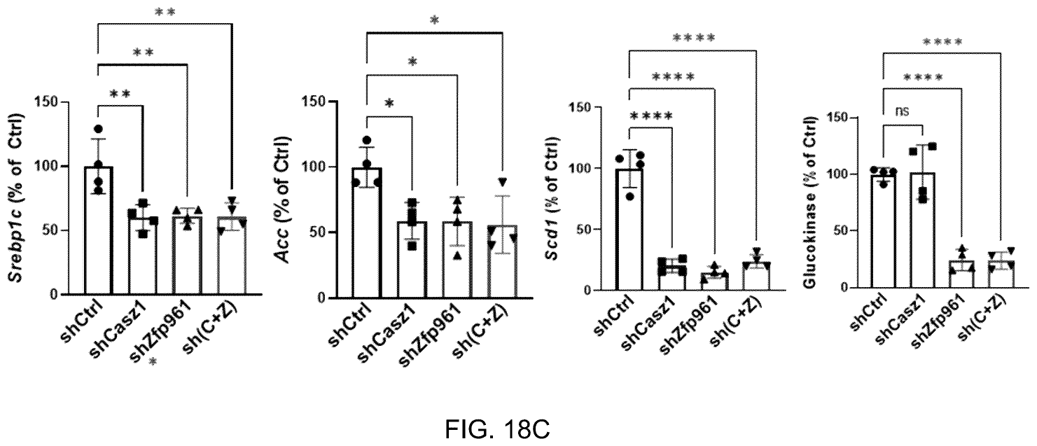

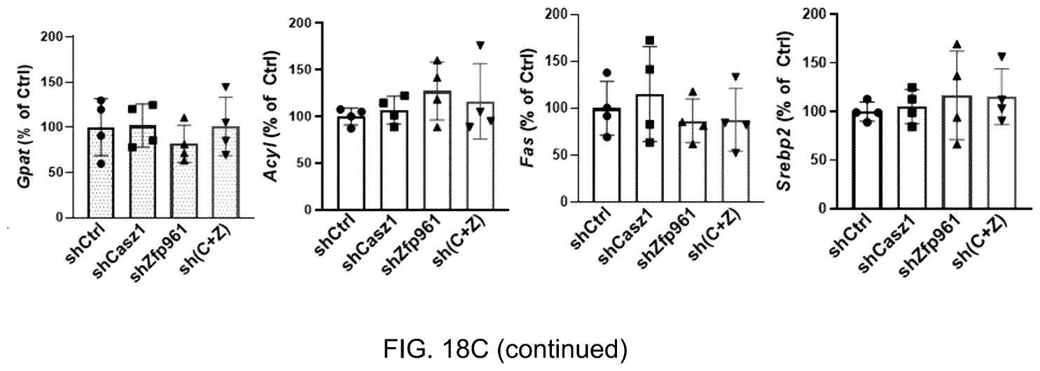

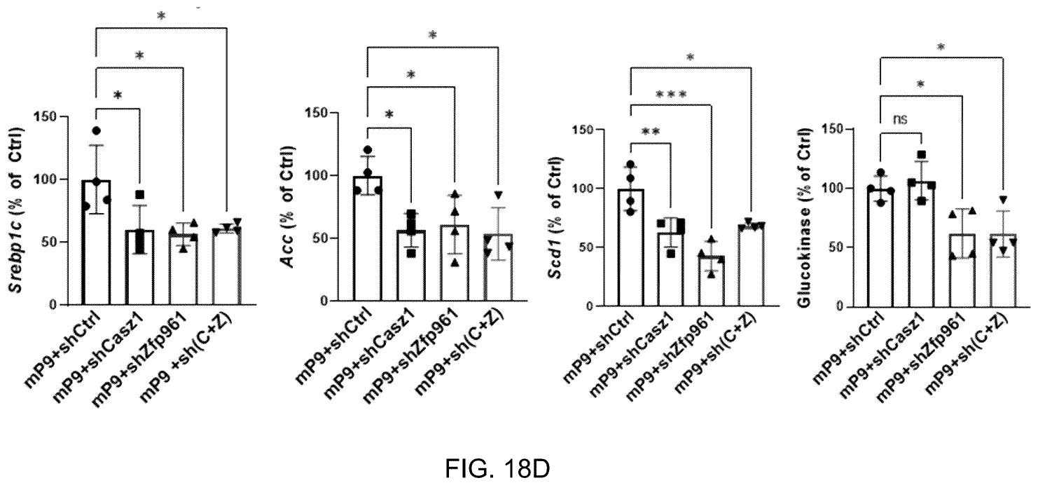

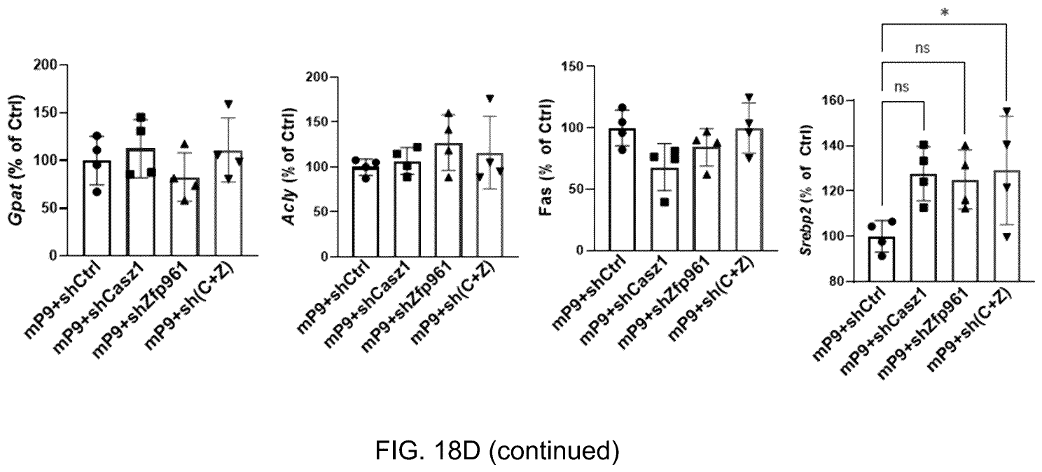

The above studies showed that shCasz1 and shZfp961 significantly reduced plasma apoB but had no significant effect on hepatic lipids and plasma AST/ALT; markers that are usually increased after reductions in apoB-Lp assembly. To identify reasons for the absence of hepatosteatosis, we quantified mRNA levels and observed no changes in fatty acid oxidation gene expression (FIG. 18A-B); however, mRNA levels of the lipogenic proteins Srebp1c, Acc1, Scd1, and glucokinase were significantly reduced (FIG. 18C-D). These results indicated that these genes may play a role in lipid synthesis. This was further tested in Huh-7 cells. Expression of miR-541-3p reduced, while anti-miR-541-3p enhanced newly synthesized cellular and secreted triglyceride and phospholipids in Huh-7 cells (FIG. 6A-B). In addition, KD of ZNF101 significantly reduced synthesis and secretion of triglycerides and phospholipids (FIG. 6C-D). SiCasz1 cells tended to reduce lipid synthesis. These studies showed that KD of these TFs reduces lipid synthesis and this might have prevented hepatosteatosis.

### Discussion of Examples

The present disclosure demonstrates that miR-541-3p concurrently reduces apoB and increases apoA1 in human hepatoma cells by regulating two different TFs, Casz1 and Znf101. Mechanistic studies show that miR-541-3p interacts with 3′-UTR of CASZ1 and ZNF101 mRNAs in Ago2 complexes and enhances their degradation. Furthermore, the disclosure demonstrates that hepatic knockdown of orthologous transcription factors in mice alter plasma lipoprotein profile and reduce atherosclerosis. Physiological studies showed that hepatic knockdown of Casz1 augments cholesterol efflux capacity by increasing apoA1 and HDL, whereas hepatic knockdown of Zfp961 reduces triglyceride production and plasma apoB-Lps. These results show that targeting Zfp961/Znf101 and Casz1 can be used to modulate plasma LDL and HDL and to reduce atherosclerosis.

It is generally believed that apoB transcription occurs constitutively and that the assembly and secretion of apoB-Lps is controlled by post-translational mechanisms (18). The present disclosure demonstrates that Znf101/Zfp961 KD reduces apoB mRNA levels in cultured cells and in mice indicating regulation at the transcriptional level. The results indicate that Znf101 interacts with the APOB promoter at +418 to enhance transcription. This site is within the DNAse 1 hypersensitive promoter region responsible for apoB expression in liver cells, but not in Hela cells (19). Furthermore, hepatic knockdown of Zfp961 reduces apoB mRNA and plasma apoB-Lps indicating that transcriptional mechanisms can alter plasma lipoproteins. In contrast to Znf101/Zfp961, KD of Casz1 has no significant effect on apoB mRNA levels, but significantly reduces hepatic triglyceride production and plasma apoB levels in mice. These results indicate that Casz1 regulates production of apoB-Lps in hepatocytes.

Several attempts have been made to inhibit hepatic apoB-Lp production to lower plasma lipids and atherosclerosis; however, these attempts have been associated with increased lipid storage in the liver. In this disclosure, reductions in plasma lipoproteins in shZfp961 treated mice were not associated with increases in hepatic lipids. The examples demonstrate significant decreases in the expression of genes involved in triglyceride and phospholipid syntheses. Furthermore, KD of Znf101 in human liver cells significantly reduced lipid synthesis. These results indicate that Znf101 reduced both apoB and lipid synthesis in liver cells to reduce plasma and hepatic lipids.

The present disclosure demonstrates that miR-541-3p mimics decrease human apoB and increase apoA1 expression by degrading Znf101 and Casz1 mRNA in human liver cells. In mouse liver cells, Zfp961 enhances the expression of apoB and plasma LDL, whereas Casz1 reduces the expression of apoA1 and plasma HDL levels. KD of these TFs decreases plasma apoB-Lps, lipogenesis and atherosclerosis, without causing hepatosteatosis or increasing plasma AST/ALT in mice. Thus, in addition to increasing miR-541-3p or by using miR-541-3p mimics, Casz1 and Znf101/Zfp961 are targets to modulate plasma lipoproteins and reduce atherosclerosis.

### Methods

Cell culture: Human hepatoma Huh-7, mouse hepatocyte AML12, and mouse macrophage J774 cells were cultured in Dulbecco's modified Eagle's medium (DMEM) containing 10% fetal bovine serum (FBS), and 1% L-glutamine in T75 flasks at 37° C. in humidified 5% CO₂ incubators(27, 28).

Screening: MiR mimics were suspended in RNase free water to obtain 2 μM stocks and 3 μL of each miR stock were added in duplicate wells to obtain a final concentration of 50 nM. To each well, we added 7 μL of Opti-MEM and 10 μL of lipofectamine RNAiMAX (Life technologies) diluted 1:20 in Serum Reduced Opti-MEM. After 20 to 30 minutes, 25,000 cells in 100 μL of Opti-MEM were added to each well. After additional 24 h, culture media were changed with fresh DMEM containing 10% fetal bovine serum. Media were changed 24 hours later and cells were incubated with DMEM containing oleic acid/BSA complex ((oleic acid (0.4 mM)/BSA (1.5%)) for 2 h. ApoB and apoAI concentrations in medium were measured by ELISA (60). Cells were used for protein measurements.

Transfection in hepatoma cells: Huh-7 or AML12 cells (2.5×10⁵) were reverse transfected in triplicate in 6 well plates using Endofectin (Genecopoeia) with the indicated doses of miRIDIAN miRNA mimics (miR-541-3p), miRIDIAN miRNA inhibitors (antimiR-541-3p), siRNAs (Origene), scramble control mimic (20 nM, Thermo Fisher Scientific), or siControl as noted in the figure legends. Media was replaced with fresh DMEM containing 10% FBS and 1% L-glutamine after 16 h of transfection. For long-term cultures, media was changed every 24 h.

ApoA1 and apoB protein quantifications in Huh-7 cells by ELISA: One day after transfections, media was replaced with 1 mL of fresh DMEM containing 10% FBS. Cells and media (last 24 h) were collected 70 h post transfection. Cells were lysed with 0.1 N NaOH to quantify total protein by BCA method. Total cell protein was used to normalize amounts of the apoA1 and apoB protein in media. To measure apoA1 and apoB protein levels in Huh-7 cells, transfected cells were lysed with 1×RIPA buffer containing protease inhibitor mixture (Sigma-Aldrich). Cell lysates were centrifuged and the supernatants were added to ELISA plates (Costar, Corning) to measure apoA1 (R&D Systems® ELISA Kits) and apoB (Mabtech ELISA Kits) in triplicate. In some samples, sandwich ELISA using 1D1 as capture antibodies was used for apoB ELISA in triplicate as previously described(27).

Quantification of mRNAs by quantitative RT-PCR: Huh-7 cells were washed with PBS, 1 mL of TRIzol was added, mixed vigorously for 30 s, and incubated for 10 min at room temperature. Similarly, 10 mg of mouse liver tissue was lysed in 1 mL of TRIzol and homogenized using a tissue homogenizer. Chloroform (200 μL) was added and mixed vigorously, left at room temperature for 5 min, centrifuged (12,000 rpm, 15 min, 4° C.), and the top aqueous phase was transferred to a new tube. Subsequently, samples were mixed with equal volumes of ice chilled isopropanol and kept at room temperature for 5 min. Samples were centrifuged (12,000 rpm, 15 min, 4° C.) and supernatants were discarded, pellets were resuspended in 1 mL of 70% ethanol, centrifuged (12,000 rpm, 15 min, 4° C.), and supernatants were discarded. Pellets were dried in 37° C. incubator for 10 min and dissolved in molecular biology grade water.

First strand cDNA was synthesized using 1 μg of RNA with the Applied Biosystems™ High-Capacity cDNA Reverse Transcription Kit was used for quantitative RT-PCR (PowerTrack SYBR Green Master Mix) in triplicate and the Ct values for each mRNA were normalized to 18S. For miR quantification, 100 ng of RNA and miR specific primers were used for cDNA synthesis using the TaqMan MicroRNA Reverse Transcription kit (Applied Biosystems, 4366597). MiR-541-3p levels were quantified using the Ct method after normalization with RNU44 or U6 and are presented as arbitrary units.

Immunoblotting: Huh-7 cells (from 1 well of a 6 well plate) were mixed with 200 μL of cold 1×RIPA buffer and vortexed. After complete cell lysis, lysates were centrifuged (1000 rpm for 10 min at 4° C.). Total protein (25 μg) was resolved on 6% and 12% acrylamide gels for apoB and apoA1, respectively. Liver tissue (50 mg) was homogenized in 1 mL of buffer K (1 mM Tris-Cl, 1 mM EGTA, and 1 mM MgCl₂, pH 7.6) containing protease inhibitor mixture. Proteins (˜25 μg) were resolved on SDS-PAGE gels, transferred to nitrocellulose blotting membranes by semi-dry transfer method and membranes were blocked with 5% dry milk powder in Tris-buffered saline (TBS) for 1 h. After washing with TBS containing 0.1% Tween® 20 detergent (TBST), membranes were incubated with 1:1000 dilution of primary polyclonal antibodies against mouse/human apoA1 and apoB or a rabbit antibody to human/mouse β-actin overnight at 4° C. The next day, membranes were washed and incubated with alkaline phosphatase conjugated secondary antibody (1:10,000 dilution) for 2 h, washed, substrate was added and bands were visualized using Storm 860 Molecular Imager.

Luciferase assay: Plasmids expressing Gaussia luciferase with ZNF101-3′-UTR, Casz1-3′-UTR, apoA1-promoter, or apoB-promoter (GLuc/SEAP dual-reporter vector system) were obtained from Genecopiea. Huh-7 cells were first forward transfected with 5 μg of these plasmids. After 16 h of transfection, cells were trypsinized and equally distributed into wells and reverse transfected in triplicate with miR-541-3p, antimiR-541-3p, or different indicated siRNAs. Luciferase activity was measured in media after 8 h of reverse transfection using a kit. Luciferase activity was normalized with SEAP (secreted alkaline phosphatase) activity.

Ago2 precipitation: Huh-7 cells transfected (n=3) with miR-541-3p or antimiR-541-3p were collected in buffer K and sonicated to lyse the cells. Cell lysates were centrifuged (10000 rpm, 10 min, 4° C.), supernatants were collected and incubated for 1 h with IgG for preclearing. After centrifugation, supernatants were mixed with protein A/G plus agarose beads for 1 h at 4° C. This mixture was centrifuged (800 rpm, 1 min) and supernatants were incubated with 1:100 dilution of human Ago2 antibody overnight at 4° C. Next day, mixtures were incubated with agarose beads for 1 h, and centrifuged (800 rpm, 1 min). Supernatants were discarded and pellets were processed for RNA isolation using TRIzol method.

Actinomycin D study: After 16 h of miR-541-3p (20 nM) transfection, Huh-7 cells were treated with 10 μg ml⁻¹ of actinomycin D in triplicate to analyze post-transcriptional degradation of different mRNAs. Cells were collected every 2 h and RNA was isolated using TRIzol. Isolated RNAs were used to quantify expression levels of apoA1, apoB, Casz1, and ZNF101 transcript levels.

De novo lipogenesis: Huh-7 cells (n=4) were reverse transfected with control miR, miR/antimiR-541-3p (20 nM) in 12-well plates. After 8 h of transfection, cells were washed three times with PBS and incubated with 500 μL of serum free media containing ³[H]-glycerol (5 μCi/mL) for 16 h. Media was collected and processed for lipid isolation using the chloroform:methanol method(29). Cells were washed three times with PBS and incubated overnight with 1 mL of isopropanol at 4° C. Isopropanol was collected and dried by vacuum centrifugation. Dried lipids were dissolved in 25 μL of chloroform and applied to thin layer chromatography (TLC) plates (Whatman, aluminum plates coated with silica, 4420221) and separated using petroleum ether:diethyl ether:acetic acid (70:30:1) solvent mixture. TLC plates were exposed to iodine vapors for 2-5 min, triglyceride and phospholipid (PL) bands were marked with pencil, excised, placed in scintillation vial and 5 mL of ScintiSafe™ Econo 2 Cocktail was added. Each sample was counted in liquid scintillation counter (Wallac 1409) for 5 min to measure disintegrations per minute (dpm).

Knockdown of TFs in mice: Female C57Bl/6 mice (n=17, 2.5-month-old) were divided into 4 groups and were intravenously transduced with different viruses expressing specific shRNA (2.5×10¹¹ genome copies) as follows: shControl (4 mice), shCasz1 (4 mice), shZfp961 (4 mice), and shCasz1+shZfp961 (5 mice). These mice were fed a Western diet (Envigo TD.88137 contains 15.2% protein, 42.7% carbohydrate, and 42% fat kcal as well as cholesterol 1.5 g/kg) for 2 months. In a separate experiment, male mice were transduced with these shRNAs along with adeno-associated virus expressing a gain-of-function mutant of mouse PCSK9 (AAV8-D377Y-mPCSK9, 1.0×10¹¹ genome copies) and fed a Western diet for 4 months. Plasma was collected at different times to document changes in plasma cholesterol and triglyceride levels. At the end, mice were fasted overnight, euthanized, and tissues were collected. In these mice, we also evaluated atherosclerosis, by dissecting and examining aortas, as previously described(30).

Plasma and tissue lipid measurements: Mice were fasted overnight (15 h) and blood was collected in EDTA-rinsed tubes from retro-orbital venous plexus. Blood was centrifuged (7,000 rpm, 10 min), plasma was collected and stored at −80° C. Total plasma cholesterol and triglyceride concentrations were enzymatically measured in triplicates using kits. HDL-C was measured after precipitating apoB-lipoproteins using equal volumes of HDL cholesterol reagent (Pointe Scientific). LDL-C values were determined by subtracting the concentrations of HDL-C from total lipids. For hepatic lipid measurements, liver pieces (˜50 mg) were homogenized in 1 mL of buffer K, and 200 μL was subjected to lipid extraction(29, 31). Plasma ALT (Cayman) and AST (Sigma) were measured in triplicates using kits according to the manufacturer's instructions.

Fast performance liquid chromatography (FPLC): Plasma samples (200 μL) were loaded onto AKTA pure FPLC column [Superose™ 6 Increase 10/300 GL FPLC column (GE Healthcare). Lipoproteins were eluted with PBS and 0.25 mL fractions were collected at a flow rate of 0.4 mL/min.

Cholesterol efflux: Mouse macrophage J774 cells were seeded in 12-well plates at a density of 75×10³ cells/well and cultured in humidified incubator (37° C., 5% CO₂). After 15 h, 250 μL of DMEM complete media containing 0.25 μCi [³H]-cholesterol+5 μg acetylated LDL (Ac-LDL) were added in each well and incubated. After 48 h, cells were examined under the microscope to ensure healthy appearance and about 80% confluency, media was removed, and cells were washed three times with PBS. Next, 500 μL of Opti-MEM media containing 2 μM LXR agonist TO-901317 was added into each well. After 18 h, media was removed and cells were gently washed with PBS. Next, Opti-MEM (500 μL) containing acceptor (HDL-C, 5 μg) or (plasma, 10 μL) in triplicate were added into each well. One set of wells received Opti-MEM only for background. After 4 h, different media was collected and centrifuged at 13,000 rpm for 10 min. Plates were kept in the freezer for 1 h, prior to adding 500 μL dH₂O to each well, and incubated on a shaker at 4° C. for 1 h to detach cells. Cells were pipetted up and down to break up cell clumps and obtain homogeneous suspensions. Media (100 μL) and cells (100 μL) were transferred to 7 mL scintillation vials, 5 mL of Insta-gel Plus (PerkinElmer) was added, mixed well, and [³H] dpm was measured in a scintillation counter. The following formula was used to measure cholesterol efflux from cells to the acceptor: % cholesterol efflux=(media count−background)/(media count−background)+(cell counts−background)×100.

Hepatic triglyceride production: Mice were fasted for 16 h and then 1.5 mg/gm (in 500 μL PBS) of poloxamer 407 (P407) was injected intraperitoneally. Plasma was collected at the indicated time points. Triglycerides were measured using a kit. Rates of triglyceride production were measured between 2 and 4 h.

Intestinal lipid absorption studies: Mice were fasted overnight, then 100 μL of olive oil was administered by intragastric gavage. Blood samples were collected from retro-orbital venous plexus at different times. Plasma triglyceride levels were measured using kits.

Aortic plaque analyses: Aortic arches were dissected and exposed for photography and quantified with ImageJ. Aortic fatty streaks were visualized with Oil Red O staining.

Statistics: All data are presented as mean±SD. Statistical significance (P<0.05) was determined using Student's t-test, one-way ANOVA, or two-way ANOVA (GraphPad Prism), and significant differences are represented as * P<0.05; **P<0.01; ***P<0.001; ****P<0.0001.

| Diets | % Fat | Company, ID |
|---|---|---|
| Chow | 13% | Lab Diet, 5053 |
| Western | 45% | Envigo, TD.88137 |
| Obesogenic | 60% | Research Diets, D12492 |

siRNAs oligo

| | |
|---|---|
| Universal scrambled | CGUUAAUCGCGUAUAAUACGCGUAT |
| negative control | (SEQ ID NO: 118) |
| ZNF101 Human siRNA | GAAGUCUAAGGUAAUUUGAAAAAGU |
| Oligo Duplex | AA (SEQ ID NO: 119) |
| CASZ1 Human siRNA | UUUGGAGAAGGUUGACUUCAGGUAC |
| Oligo Duplex | UC (SEQ ID NO: 120) |
| Casz1 Mouse siRNA | CGUGAAGGAUCCCAGUAAUGAAUCA |
| Oligo Duplex | (SEQ ID NO: 121) |
| Zfp961 Mouse siRNA | ACACUGCCAGUCAAUCAUCCCUGAA |
| Oligo Duplex | (SEQ ID NO: 122) |

Reagents

| Items | Vendor | Catalog # |
|---|---|---|
| Hsa-miR-541-3p | Thermo Fisher | 4464066, Assay |
| | Scientific | ID#MC13119 |
| Hsa-AntimiR-541-3p | Thermo Fisher | 4464084, Assay |
| | Scientific | ID# MH13119 |
| Hsa-miR-541-5p | Thermo Fisher | 4464066, Assay |
| | Scientific | ID#MC12516 |
| miRNA Negative Control | Thermo Fisher | 4464058 |
| | Scientific | |
| TaqMan ™ miR-541-3p Assay | Thermo Fisher | 4427975, Assay |
| | Scientific | ID#002201 |
| TaqMan ™ miR-U6 Assay | Thermo Fisher | 4427975, Assay |
| | Scientific | ID#001973 |
| TaqMan ™ MicroRNA Reverse Transcription | Thermo Fisher | 4366596 |
| Kit | Scientific | |
| TaqMan ™ Universal Master Mix II, no UNG | Thermo Fisher | 4440043 |
| | Scientific | |
| PowerTrack SYBR Green Master Mix | Thermo Fisher | A46012 |
| | Scientific | |
| High-Capacity cDNA Reverse Transcription | Thermo Fisher | 4374967 |
| Kit | Scientific | |
| TRIzol ™ Reagent | Thermo Fisher | 15596026 |
| | Scientific | |
| ELISA Pro: Human apoB | MABTECH | 3715-1HP-20 |
| Human Apolipoprotein A-I/ApoA1 DuoSet | R&D system | DY3664-05 |
| ELISA | | |
| miRNA Target clone control vector | Genecopoeia | CmiT000001-MT05 |
| miRNA 3′UTR target expression clone for | Genecopoeia | HmiT071405-MT05 |
| Human ZNF101 | | |
| miRNA 3′UTR target expression clone for | Genecopoeia | HmiT105330-MT05 |
| Human CASZ1 | | |
| Custom promoter reporter clone for Human | Genecopoeia | CS-HPRM30361- |
| APOB | | PG04-02 |
| Promoter reporter clone for Human APOA1 | Genecopoeia | HPRM25646-PG04 |
| Zfp961-AAVPrime ™ Purified AAV Particles | Genecopoeia | AA08-MSH074798- |
| | | AV03 |
| Casz1-AAVPrime ™ Purified AAV Particles | Genecopoeia | AA08-MSH034915- |
| | | AV03 |
| AAVPrime ™ Purified AAV Particles for | Genecopoeia | AA08- |
| shRNA scrambled control | | CSHCTR001-AV03 |
| AAV8-D377y-mPCSK9 (5e12 gc) | Vector biosystems Inc | 7600 |
| Secrete-Pair Dual Luminescence Assay | | LF031 |
| Apolipoprotein A-I/ApoA1 Antibody | NOVUS | NBP2-15429 |
| Apolipoprotein B (APOB), Polyclonal | MyBiosource | MBS2006107 |
| Antibody | | |
| Anti-Argonaute-2 antibody | Abcam | ab186733 |
| Pointe Scientific Triglycerides Liquid Reagent | Pointe Scientific | 23-666-411 |
| Pointe Scientific Cholesterol Liquid Reagents | Pointe Scientific | 23-666-200 |
| EndoFectin ™ Max transfection Reagent | Genecopoeia | EF013 |
| Protease Inhibitor Cocktail | Sigma Aldrich | P1860 |
| RIPA buffer | Thermo Fisher | J63306 |
| | Scientific | |
| Poloxamer 40716758 | Sigma Aldrich | 40716758 |
| Olive oil | Sigma Aldrich | 01514 |
| T0901317, LXR agonist | Abcam | ab142808 |
| ScintiSafe ™ Econo 2 Cocktail | Thermo Fisher | FISSX21-5 |
| | Scientific | |
| Cholesterol, [1,2-3H(N)] | Perkin Elmer | NET139001MC |

Human primers

| | Forward primer | Reverse primer |
|---|---|---|
| Gene name | | |
| ZNF101 | GGAAGCCGGAAATGGACTCA | CATTGGATTCCGACCGAGGC |
| | G (SEQ ID NO: 2) | (SEQ ID NO: 3) |
| MAFK | GCACACATGGCAGAGAGAGT | GAGTCCTGCTCACCGTCAAA |
| | (SEQ ID NO: 4) | (SEQ ID NO: 5) |
| NFATC3 | GCTCTCATGTCCAGCTCCTT | AGTTGGTGGTGGACACAAGG |
| | T (SEQ ID NO: 6) | (SEQ ID NO: 7) |
| RELA | TCCAGTGTGTGAAGAAGCGG | TCCCCACGCTGCTCTTCTAT |
| | (SEQ ID NO: 8) | (SEQ ID NO: 9) |
| ZNF275 | AAGTCCTTCCGAGGGGTCAA | GAAGGCCGCAGGCGTAG |
| | (SEQ ID NO: 123) | (SEQ ID NO: 124) |
| ELK4 | TCACGAGCAGTGATCCAAGC | AAGAGCGAGCAAGCTACCTG |
| | (SEQ ID NO: 10) | (SEQ ID NO: 11) |
| HSF1 | GAAGCAGCTGGTGCACTACA | CTGTCAGCAGGGAGATGGTG |
| | (SEQ ID NO: 12) | (SEQ ID NO: 13) |
| ZNF442 | ACCTCATGTGTTGTAAAAGC | CTATGTAGGCAGGCCTGTGA |
| | TATGT (SEQ ID NO: | (SEQ ID NO: 15) |
| | 14) | |
| ZSCAN22 | GCGCAAGTTGGCTAGTCTCT | GGAAGCTGTCCTCTTCCCAC |
| | (SEQ ID NO: 16) | (SEQ ID NO: 17) |
| ZNF746 | GGACCCTGAAGCTCAACACA | CTTTCCCAGGCTCCTTCCTG |
| | (SEQ ID NO: 18) | (SEQ ID NO: 19) |
| CASZ1 | CCGAGGGTGTCTACATGGTG | CCCGTCCGAATCCTTCTCC |
| | (SEQ ID NO: 20) | (SEQ ID NO: 21) |
| HNF4A | GCAATGACACGTCCCCATCA | TCGAGGCACCGTAGTGTTTG |
| | (SEQ ID NO: 22) | (SEQ ID NO: 23) |
| ZNF771 | AGGGGGTGGGGCTATATGTT | CAGGCATCTTGGTGTCCTGA |
| | C (SEQ ID NO: 24) | G (SEQ ID NO: 25) |
| IRF3 | ACACATACTGGGCAGTGAGC | CTACAATGAAGGGCCCCAGG |
| | (SEQ ID NO: 26) | (SEQ ID NO: 27) |
| THRB | TGCGCTTGGAAAAGAGACCT | AATTACCAGTGCCCTGGAGC |
| | (SEQ ID NO: 28) | (SEQ ID NO: 29) |
| NR4A3 | TGCGTCCAAGCCCAATATAG | GGTGTATTCCGAGCTGTATG |
| | C (SEQ ID NO: 30) | TCT (SEQ ID NO: 31) |
| ZBTB4 | TCCCTTTTGCACTGAGGCTT | CCTTCGATTCGCAGAGCAGA |
| | (SEQ ID NO: 32) | (SEQ ID NO: 33) |
| ZNf100 | AGGGGCCATTGACGTTTAGG | AGGGCTCTTTTCCTTGCTCC |
| | (SEQ ID NO: 34) | (SEQ ID NO: 35) |
| RARA | CACACACCTGAGCAGCATCA | TCAAGAGCCGGTCCTTTGGT |
| | C (SEQ ID NO: 36) | (SEQ ID NO: 37) |
| ZSCAN16 | TAAGGCAGAGGACCATTACT | TGCATCCTGATAGCAGAGCT |
| | GG (SEQ ID NO: 38) | T (SEQ ID NO: 39) |
| ZNF726 | ATGAACCCCCAGGTAGGTGA | TTCAGGCTTTCCAGAAACGG |
| | (SEQ ID NO: 40) | T (SEQ ID NO: 41) |
| YY1 | ACGGCTTCGAGGATCAGATT | TGACCAGCGTTTGTTCAATG |
| | C (SEQ ID NO: 42) | T (SEQ ID NO: 43) |
| ZNF4471 | GTCCTTCGGATGAGAGCGTC | AGCAGGGTTCATCCATTGCC |
| | (SEQ ID NO: 44) | (SEQ ID NO: 45) |
| ZNF81 | GTTCTCAGCCGGGGTTTGAT | CTGCTGGGGTCAGAAGGAAG |
| | (SEQ ID NO: 46) | (SEQ ID NO: 47) |
| SP6 | CTTCCTTGCTGAGAGGGTTG | TAGGATGGCCACTGGAAAAG |
| | (SEQ ID NO: 125) | (SEQ ID NO: 126) |
| FOXI1 | GGAAGTCACCAGTGGCCTTA | GGTGGGAGGATTTAGGGGTA |
| (NM_012188.5) | (SEQ ID NO: 48) | (SEQ ID NO: 49) |
| ONECUT3 | AAGGAACCTCCTCCCTGAAA | GCACAAATCCTTGGGAGAAA |
| | (SEQ ID NO: 50) | (SEQ ID NO: 51) |
| ZNF418 | GGGTGTGTTTCTGCGGTTTG | CCTCTGTGCAGCAAGAAGGA |
| | (SEQ ID NO: 52) | (SEQ ID NO: 53) |
| MTP | | |
| ABCA1 | AACAGTTTGTGGCCCTTTTG | AGTTCCAGGCTGGGGTACTT |
| | (SEQ ID NO: 54) | (SEQ ID NO: 55) |
| ApoA1 | AGAGACTGCGAGAAGGAGGT | TCTCTGCCGCTGTCTTTGAG |
| | (SEQ ID NO: 56) | (SEQ ID NO: 57) |
| ApoB | TGTCAGTACACACTGGACGC | TCAAATGCGAGGCCCATCTT |
| | (SEQ ID NO: 58) | (SEQ ID NO: 59) |
| SDM_apoa1 | GCGTGATCAAcgcTAAGTGT | ACAGGTGTGACTGGATCTC |
| luciferase | GAACAATGCAAAGG (SEQ | (SEQ ID NO: 61) |
| promoter | ID NO: 60) | |
| plasmid | | |
| SDM_ApoB | AAGGCATCTGataATGGGGC | ACTTGGACAGACCAGGCT |
| luciferase | GTG (SEQ ID NO: 62) | (SEQ ID NO: 63) |
| promoter | | |
| plasmid with | | |
| mutated bases | | |

Mice primers

| | | |
|---|---|---|
| Casz1 | CCTCCAACTGCCTGAGCTAC | CGGGGTCCTAGAGATTCCTC |
| | (SEQ ID NO: 64) | (SEQ ID NO: 65) |
| Zfp961 | TGACCCATGTGGAGAAACGG | ACAGTGCCGGTAACTGAAGG |
| | (SEQ ID NO: 66) | (SEQ ID NO: 67) |
| Zfp101 | GGTGAATGTGAACGTGGTTG | CCCAGCATTCCTCAGGTTTA |
| | (SEQ ID NO: 68) | (SEQ ID NO: 69) |
| Apoa1 | GGCCGTGGCTCTGGTCTT | AGCGTGGTGAAAGGGCTTAT |
| | (SEQ ID NO: 70) | (SEQ ID NO: 71) |
| ApoB | TCCATATTCCAGACAACCTC | GTTTATTTTGTTCCTGTTCA |
| | TTC (SEQ ID NO: 72) | TTGTGT (SEQ ID NO: |
| | | 73) |
| HNF4α | ACAGGAGAGGGTCAGAAGCA | ATGTTTGCACAACCACAGGA |
| | (SEQ ID NO: 74) | (SEQ ID NO: 75) |
| PPARα | CACGCATGTGAAGGCTGT | GCTCCGATCACACTTGTCG |
| | (SEQ ID NO: 76) | (SEQ ID NO: 77) |
| PGCa1α | CTGTCGAGTCTGTTGGAGCA | GGGAATGTCAATGCCTGAGT |
| | (SEQ ID NO: 78) | (SEQ ID NO: 79) |
| C/EBP | TTACAACAGGCCAGGTTTCC | CTCTGGGATGGATCGATTGT |
| | (SEQ ID NO: 80) | (SEQ ID NO: 81) |
| MTP | CACACAACTGGCTCTCTCAT | TGCCCCCATCAAGAAACACT |
| | TAAAT (SEQ ID NO: | (SEQ ID NO: 83) |
| | 82) | |
| ABCA1 | AACAGTTTGTGGCCCTTTTG | AGTTCCAGGCTGGGGTACTT |
| | (SEQ ID NO: 84) | (SEQ ID NO: 85) |
| LDLR | GAAAAGGCTACTGGCTGTGC | CCAGGACCCGGTCAGTAGTA |
| | (SEQ ID NO: 86) | (SEQ ID NO: 87) |
| SREBP2 | CCATCTTCCCCTCTCTTTCC | AGGGAAGATCCTGGGAGAAA |
| | (SEQ ID NO: 88) | (SEQ ID NO: 89) |
| SCD1 | CTTCAAGGGCAGTTCTGAGG | CAATGGTTTTCATGGCAGTG |
| | (SEQ ID NO: 90) | (SEQ ID NO: 91) |
| Gluco- | CTTTCCAGGCCACAAACATT | TGAGTGTTGAAGCTGCCATC |
| kinase | (SEQ ID NO: 92) | (SEQ ID NO: 93) |
| mGpat | AGCAAGTCCTGCGCTATCAT | CTCGTGTGGGTGATTGTGAC |
| | (SEQ ID NO: 94) | (SEQ ID NO: 95) |
| ACLY | AGGTCTCTCTGCAGCCATGT | AAGCTTTCCTCGACGTTTGA |
| | (SEQ ID NO: 96) | (SEQ ID NO: 97) |
| ACC | GCCTCTTCCTGACAAACGAG | TGACTGCCGAAACATCTCTG |
| | (SEQ ID NO: 98) | (SEQ ID NO: 99) |
| SREBP1c | AGGTGTATTTGCTGGCTTGG | AGAGATGACTAGGGAACTGT |
| | T (SEQ ID NO: 100) | GTGT (SEQ ID NO: |
| | | 101) |
| FAS | CTCAGTGTGCCCACCTAG | GCACTTGCTTGATGCAATCT |
| | (SEQ ID NO: 102) | (SEQ ID NO: 103) |
| UCP1 | CGTGAAGGTCAGAATGCAA | GCATTGTAGGTCCCCGTG |
| | (SEQ ID NO: 104) | (SEQ ID NO: 105) |
| UCP2 | GCGTTCTGGGTACCATCCTA | GCTCTGAGCCCTTGGTGTAG |
| | (SEQ ID NO: 106) | (SEQ ID NO: 107) |
| CPT1 | GACTCCGCTCGCTCATTC | TCTGCCATCTTGAGTGGTGA |
| | (SEQ ID NO: 108) | (SEQ ID NO: 109) |
| HNF4α | ACAGGAGAGGGTCAGAAGCA | ATGTTTGCACAACCACAGGA |
| | (SEQ ID NO: 110) | (SEQ ID NO: 111) |
| PPARα | CACGCATGTGAAGGCTGT | GCTCCGATCACACTTGTCG |
| | (SEQ ID NO: 112) | (SEQ ID NO: 113) |
| PGCa1α | CTGTCGAGTCTGTTGGAGCA | GGGAATGTCAATGCCTGAGT |
| | (SEQ ID NO: 114) | (SEQ ID NO: 115) |
| C/EBP | TTACAACAGGCCAGGTTTCC | CTCTGGGATGGATCGATTGT |
| | (SEQ ID NO: 116) | (SEQ ID NO: 117) |

Supplementary Table I. The effect of 1237 miRs on apoB and apoAI secretion in Huh7 cells. Medium apoB and apoAI were normalized against protein levels and calculated to percentages of Scr control. The sequences associated with the accession numbers below are available from www.mirbase.org.

| | Mature | apoB | | apo AI | |
|---|---|---|---|---|---|
| Mature Name | Accession | plate1 | plate2 | plate1 | plate2 |
| hsa-let-7a-2-3p | MIMAT0010195 | 110.79 | 94.94 | 114.44 | 102.87 |
| hsa-let-7a-3p | MIMAT0004481 | 112.31 | 86.33 | 73.30 | 59.51 |
| hsa-let-7a-5p | MIMAT0000062 | 35.73 | 56.36 | 72.34 | 83.45 |
| hsa-let-7b-3p | MIMAT0004482 | 105.54 | 109.86 | 90.36 | 91.48 |
| hsa-let-7b-5p | MIMAT0000063 | 77.29 | 57.43 | 107.87 | 115.59 |
| hsa-let-7c | MIMAT0000064 | 67.69 | 62.76 | 78.38 | 70.12 |
| hsa-let-7c* | MIMAT0004483 | 89.15 | 94.94 | 114.44 | 107.62 |
| hsa-let-7d-3p | MIMAT0004484 | 158.43 | 246.59 | 91.70 | 95.20 |
| hsa-let-7d-5p | MIMAT0000065 | 32.34 | 31.45 | 62.74 | 39.59 |
| hsa-let-7e-3p | MIMAT0004485 | 125.04 | 144.66 | 96.92 | 102.60 |
| hsa-let-7e-5p | MIMAT0000066 | 78.48 | 67.11 | 89.71 | 77.22 |
| hsa-let-7f-1-3p | MIMAT0004486 | 122.09 | 92.95 | 100.81 | 95.83 |
| hsa-let-7f-2-3p | MIMAT0004487 | 138.71 | 148.89 | 93.54 | 97.85 |
| hsa-let-7f-5p | MIMAT0000067 | 72.94 | 71.62 | 60.78 | 71.53 |
| hsa-let-7g-3p | MIMAT0004584 | 69.96 | 67.38 | 110.83 | 99.48 |
| hsa-let-7g-5p | MIMAT0000414 | 55.08 | 35.56 | 91.47 | 83.59 |
| hsa-let-7i-3p | MIMAT0004585 | 86.46 | 92.51 | 108.30 | 108.19 |
| hsa-let-7i-5p | MIMAT0000415 | 35.04 | 32.74 | 64.23 | 51.90 |
| hsa-miR-1 | MIMAT0000416 | 144.14 | 142.57 | 125.78 | 120.96 |
| hsa-miR-100-3p | MIMAT0004512 | 102.52 | 82.20 | 100.81 | 113.61 |
| hsa-miR-100-5p | MIMAT0000098 | 107.18 | 111.45 | 102.86 | 80.38 |
| hsa-miR-101-3p | MIMAT0000099 | 107.83 | 106.29 | 94.22 | 81.17 |
| hsa-miR-101-5p | MIMAT0004513 | 47.51 | 60.73 | 67.47 | 70.52 |
| hsa-miR-103a-2-5p | MIMAT0009196 | 87.04 | 114.00 | 94.41 | 92.61 |
| hsa-miR-103a-3p | MIMAT0000101 | 93.20 | 105.24 | 79.17 | 81.24 |
| hsa-miR-103b | MIMAT0007402 | 91.33 | 92.95 | 125.87 | 122.49 |
| hsa-miR-105-3p | MIMAT0004516 | 162.70 | 201.90 | 107.31 | 122.44 |
| hsa-miR-105-5p | MIMAT0000102 | 89.81 | 80.09 | 82.59 | 115.36 |
| hsa-miR-106a-3p | MIMAT0004517 | 100.65 | 91.42 | 71.76 | 71.72 |
| hsa-miR-106a-5p | MIMAT0000103 | 130.76 | 120.29 | 91.17 | 123.07 |
| hsa-miR-106b-3p | MIMAT0004672 | 42.28 | 41.93 | 101.74 | 71.50 |
| hsa-miR-106b-5p | MIMAT0000680 | 105.69 | 78.40 | 119.00 | 137.63 |
| hsa-miR-107 | MIMAT0000104 | 152.95 | 152.07 | 93.21 | 120.04 |
| hsa-miR-10a-3p | MIMAT0004555 | 112.50 | 89.12 | 107.03 | 85.44 |
| hsa-miR-10a-5p | MIMAT0000253 | 111.97 | 124.33 | 90.01 | 79.79 |
| hsa-miR-10b-3p | MIMAT0004556 | 138.06 | 125.51 | 96.13 | 76.86 |
| hsa-miR-10b-5p | MIMAT0000254 | 110.05 | 109.81 | 111.83 | 81.95 |
| hsa-miR-1178-3p | MIMAT0005823 | 116.70 | 96.58 | 101.86 | 91.31 |
| hsa-miR-1179 | MIMAT0005824 | 113.16 | 124.38 | 102.85 | 112.60 |
| hsa-miR-1180 | MIMAT0005825 | 95.23 | 86.33 | 78.32 | 84.34 |
| hsa-miR-1181 | MIMAT0005826 | 122.09 | 106.78 | 105.94 | 82.51 |
| hsa-miR-1182 | MIMAT0005827 | 138.79 | 128.61 | 126.82 | 128.87 |
| hsa-miR-1183 | MIMAT0005828 | 52.19 | 45.32 | 64.36 | 59.02 |
| hsa-miR-1184 | MIMAT0005829 | 144.95 | 126.62 | 95.92 | 122.56 |
| hsa-miR-1184 | MIMAT0005829 | 129.95 | 123.14 | 91.66 | 112.83 |
| hsa-miR-1185-5p | MIMAT0005798 | 94.13 | 79.12 | 104.80 | 114.24 |
| hsa-miR-1193 | MIMAT0015049 | 118.45 | 81.94 | 95.19 | 81.56 |
| hsa-miR-1197 | MIMAT0005955 | 113.14 | 88.51 | 119.20 | 99.16 |
| hsa-miR-1200 | MIMAT0005863 | 28.99 | 31.74 | 145.08 | 155.96 |
| hsa-miR-1201 | MIMAT0005864 | 82.47 | 111.85 | 83.60 | 78.97 |
| hsa-miR-1202 | MIMAT0005865 | 75.58 | 65.19 | 112.88 | 98.47 |
| hsa-miR-1203 | MIMAT0005866 | 135.30 | 129.60 | 134.57 | 130.80 |
| hsa-miR-1204 | MIMAT0005868 | 85.73 | 109.22 | 106.35 | 102.34 |
| hsa-miR-1205 | MIMAT0005869 | 83.53 | 96.30 | 108.09 | 106.54 |
| hsa-miR-1206 | MIMAT0005870 | 62.56 | 77.76 | 63.54 | 58.24 |
| hsa-miR-1207-3p | MIMAT0005872 | 61.40 | 60.18 | 113.17 | 95.95 |
| hsa-miR-1207-5p | MIMAT0005871 | 112.87 | 117.90 | 151.94 | 123.51 |
| hsa-miR-1208 | MIMAT0005873 | 114.87 | 109.58 | 151.91 | 161.83 |
| hsa-miR-122-3p | MIMAT0004590 | 114.53 | 99.16 | 87.63 | 104.83 |
| hsa-miR-1224-3p | MIMAT0005459 | 84.09 | 100.83 | 93.28 | 100.31 |
| hsa-miR-1224-5p | MIMAT0005458 | 86.15 | 78.45 | 104.32 | 102.15 |
| hsa-miR-1225-3p | MIMAT0005573 | 96.94 | 99.01 | 112.33 | 107.03 |
| hsa-miR-1225-5p | MIMAT0005572 | 131.99 | 140.53 | 102.55 | 92.98 |
| hsa-miR-122-5p | MIMAT0000421 | 123.66 | 141.89 | 94.10 | 115.04 |
| hsa-miR-1226-3p | MIMAT0005577 | 96.52 | 85.57 | 98.76 | 104.73 |
| hsa-miR-1226-5p | MIMAT0005576 | 136.29 | 122.54 | 111.73 | 135.94 |
| hsa-miR-1227-3p | MIMAT0005580 | 102.71 | 126.71 | 126.08 | 149.56 |
| hsa-miR-1228-3p | MIMAT0005583 | 166.12 | 150.46 | 140.76 | 137.86 |
| hsa-miR-1228-5p | MIMAT0005582 | 101.82 | 104.19 | 103.67 | 155.07 |
| hsa-miR-1229-3p | MIMAT0005584 | 83.85 | 99.11 | 105.37 | 102.92 |
| hsa-miR-1231 | MIMAT0005586 | 140.49 | 134.25 | 75.53 | 78.78 |
| hsa-miR-1233-3p | MIMAT0005588 | 66.51 | 70.96 | 99.71 | 102.70 |
| hsa-miR-1233-3p | MIMAT0005588 | 76.74 | 64.70 | 109.18 | 101.08 |
| hsa-miR-1234-3p | MIMAT0005589 | 101.30 | 96.12 | 113.06 | 121.44 |
| hsa-miR-1236-3p | MIMAT0005591 | 134.26 | 123.35 | 122.18 | 115.82 |
| hsa-miR-1237-3p | MIMAT0005592 | 63.63 | 72.23 | 98.39 | 128.44 |
| hsa-miR-1238-3p | MIMAT0005593 | 107.18 | 97.60 | 85.01 | 35.11 |
| hsa-miR-1243 | MIMAT0005894 | 119.29 | 108.32 | 56.96 | 22.21 |
| hsa-miR-124-3p | MIMAT0000422 | 85.48 | 81.06 | 100.65 | 109.74 |
| hsa-miR-1244 | MIMAT0005896 | 99.83 | 80.53 | 85.69 | 101.62 |
| hsa-miR-1244 | MIMAT0005896 | 73.40 | 91.43 | 91.12 | 106.18 |
| hsa-miR-1245a | MIMAT0005897 | 141.91 | 145.35 | 113.00 | 129.59 |
| hsa-miR-124-5p | MIMAT0004591 | 39.80 | 37.58 | 93.96 | 99.34 |
| hsa-miR-1246 | MIMAT0005898 | 86.87 | 106.17 | 103.77 | 110.50 |
| hsa-miR-1247-5p | MIMAT0005899 | 95.76 | 130.95 | 105.47 | 87.89 |
| hsa-miR-1248 | MIMAT0005900 | 110.60 | 90.56 | 114.00 | 80.92 |
| hsa-miR-1249 | MIMAT0005901 | 80.02 | 96.24 | 93.06 | 108.84 |
| hsa-miR-1250 | MIMAT0005902 | 71.49 | 61.09 | 110.40 | 94.68 |
| hsa-miR-1251 | MIMAT0005903 | 121.16 | 116.00 | 107.65 | 106.62 |
| hsa-miR-1252 | MIMAT0005944 | 126.57 | 104.36 | 127.72 | 113.14 |
| hsa-miR-1253 | MIMAT0005904 | 102.13 | 80.01 | 72.53 | 84.02 |
| hsa-miR-1254 | MIMAT0005905 | 49.17 | 59.73 | 83.65 | 99.30 |
| hsa-miR-1255a | MIMAT0005906 | 88.54 | 80.66 | 107.65 | 118.68 |
| hsa-miR-1255b-5p | MIMAT0005945 | 74.20 | 67.39 | 109.32 | 98.34 |
| hsa-miR-1256 | MIMAT0005907 | 38.12 | 42.15 | 72.95 | 79.25 |
| hsa-miR-1257 | MIMAT0005908 | 197.57 | 160.70 | 135.85 | 123.64 |
| hsa-miR-1258 | MIMAT0005909 | 179.87 | 232.76 | 108.22 | 116.78 |
| hsa-miR-1259 | MIMAT0005910 | 76.42 | 94.49 | 83.16 | 92.03 |
| hsa-miR-125a-3p | MIMAT0004602 | 66.15 | 67.12 | 104.32 | 117.84 |
| hsa-miR-125a-5p | MIMAT0000443 | 88.45 | 101.98 | 97.01 | 95.28 |
| hsa-miR-125b-1-3p | MIMAT0004592 | 110.90 | 129.83 | 87.71 | 102.82 |
| hsa-miR-125b-2-3p | MIMAT0004603 | 65.00 | 51.04 | 62.15 | 88.87 |
| hsa-miR-125b-5p | MIMAT0000423 | 62.52 | 83.48 | 73.26 | 115.05 |
| hsa-miR-1260a | MIMAT0005911 | 171.48 | 169.77 | 102.52 | 111.07 |
| hsa-miR-1260b | MIMAT0015041 | 93.11 | 103.66 | 115.34 | 121.75 |
| hsa-miR-1261 | MIMAT0005913 | 76.42 | 92.95 | 100.24 | 102.82 |
| hsa-miR-1262 | MIMAT0005914 | 66.49 | 63.99 | 96.22 | 79.39 |
| hsa-miR-1263 | MIMAT0005915 | 102.52 | 103.71 | 129.86 | 137.72 |
| hsa-miR-126-3p | MIMAT0000445 | 112.30 | 124.65 | 118.57 | 101.17 |
| hsa-miR-1264 | MIMAT0005791 | 138.86 | 129.83 | 59.80 | 52.68 |
| hsa-miR-1265 | MIMAT0005918 | 134.20 | 146.73 | 105.37 | 107.89 |
| hsa-miR-126-5p | MIMAT0000444 | 113.16 | 90.56 | 103.41 | 78.35 |
| hsa-miR-1266 | MIMAT0005920 | 134.15 | 118.21 | 95.23 | 85.17 |
| hsa-miR-1267 | MIMAT0005921 | 110.76 | 101.11 | 107.61 | 108.03 |
| hsa-miR-1268a | MIMAT0005922 | 94.84 | 100.76 | 73.66 | 78.84 |
| hsa-miR-1269a | MIMAT0005923 | 59.36 | 21.49 | 80.55 | 48.38 |
| hsa-miR-1270 | MIMAT0005924 | 125.32 | 138.58 | 88.91 | 81.83 |
| hsa-miR-1270 | MIMAT0005924 | 118.58 | 138.58 | 81.45 | 81.40 |
| hsa-miR-1271-5p | MIMAT0005796 | 99.97 | 110.28 | 82.76 | 115.02 |
| hsa-miR-1272 | MIMAT0005925 | 141.70 | 139.97 | 163.23 | 134.14 |
| hsa-miR-1273a | MIMAT0005926 | 135.71 | 143.44 | 83.52 | 110.63 |
| hsa-miR-1273c | MIMAT0015017 | 130.63 | 118.27 | 106.82 | 117.69 |
| hsa-miR-1273d | MIMAT0015090 | 97.89 | 94.62 | 97.34 | 86.90 |
| hsa-miR-1273e | MIMAT0018079 | 70.09 | 81.33 | 79.73 | 77.49 |
| hsa-miR-127-3p | MIMAT0000446 | 67.87 | 73.75 | 68.92 | 79.61 |
| hsa-miR-1274a | MIMAT0005927 | 89.33 | 96.24 | 94.77 | 111.23 |
| hsa-miR-1274b | MIMAT0005938 | 109.93 | 126.62 | 111.55 | 104.07 |
| hsa-miR-1275 | MIMAT0005929 | 25.09 | 44.86 | 79.58 | 120.32 |
| hsa-miR-127-5p | MIMAT0004604 | 88.18 | 130.82 | 70.19 | 70.88 |
| hsa-miR-1276 | MIMAT0005930 | 159.21 | 175.34 | 102.31 | 120.83 |
| hsa-miR-1277-3p | MIMAT0005933 | 112.99 | 115.43 | 106.71 | 100.13 |
| hsa-miR-1278 | MIMAT0005936 | 98.64 | 97.59 | 110.61 | 119.82 |
| hsa-miR-1279 | MIMAT0005937 | 169.64 | 159.80 | 117.45 | 112.66 |
| hsa-miR-128 | MIMAT0000424 | 119.68 | 102.74 | 94.52 | 90.63 |
| hsa-miR-1280 | MIMAT0005946 | 88.40 | 80.08 | 108.55 | 113.85 |
| hsa-miR-1281 | MIMAT0005939 | 90.29 | 83.83 | 107.03 | 121.36 |
| hsa-miR-1282 | MIMAT0005940 | 118.45 | 134.23 | 110.53 | 99.21 |
| hsa-miR-1283 | MIMAT0005799 | 140.73 | 129.83 | 103.66 | 114.24 |
| hsa-miR-1284 | MIMAT0005941 | 109.30 | 90.73 | 101.22 | 99.81 |
| hsa-miR-1285-3p | MIMAT0005876 | 89.47 | 97.56 | 80.31 | 101.55 |
| hsa-miR-1286 | MIMAT0005877 | 88.16 | 82.27 | 88.36 | 106.93 |
| hsa-miR-1287 | MIMAT0005878 | 100.35 | 99.72 | 100.06 | 123.73 |
| hsa-miR-1288 | MIMAT0005942 | 125.82 | 105.24 | 84.86 | 78.06 |
| hsa-miR-1289 | MIMAT0005879 | 102.96 | 79.43 | 92.91 | 81.85 |
| hsa-miR-1290 | MIMAT0005880 | 129.92 | 136.08 | 100.39 | 110.04 |
| hsa-miR-1291 | MIMAT0005881 | 125.63 | 98.77 | 126.67 | 110.42 |
| hsa-miR-129-1-3p | MIMAT0004548 | 147.09 | 133.17 | 127.98 | 123.87 |
| hsa-miR-129-2-3p | MIMAT0004605 | 130.20 | 145.87 | 116.12 | 118.43 |
| hsa-miR-1292-5p | MIMAT0005943 | 114.12 | 114.84 | 156.17 | 136.96 |
| hsa-miR-1293 | MIMAT0005883 | 42.19 | 34.36 | 119.24 | 138.84 |
| hsa-miR-1294 | MIMAT0005884 | 36.74 | 33.06 | 62.19 | 72.23 |
| hsa-miR-1295a | MIMAT0005885 | 116.97 | 105.37 | 108.17 | 91.69 |
| hsa-miR-129-5p | MIMAT0000242 | 127.86 | 101.94 | 107.82 | 115.57 |
| hsa-miR-1296 | MIMAT0005794 | 135.30 | 130.50 | 99.05 | 87.84 |
| hsa-miR-1297 | MIMAT0005886 | 67.92 | 63.87 | 80.62 | 83.49 |
| hsa-miR-1298 | MIMAT0005800 | 131.10 | 120.16 | 115.11 | 128.44 |
| hsa-miR-1299 | MIMAT0005887 | 177.44 | 151.54 | 96.24 | 102.34 |
| hsa-miR-1300 | MIMAT0005888 | 130.16 | 110.81 | 72.64 | 78.04 |
| hsa-miR-1301 | MIMAT0005797 | 99.24 | 97.89 | 106.40 | 82.57 |
| hsa-miR-1302 | MIMAT0005890 | 98.92 | 120.50 | 76.87 | 108.47 |
| hsa-miR-1302 | MIMAT0005890 | 109.87 | 107.42 | 94.54 | 110.95 |
| hsa-miR-1303 | MIMAT0005891 | 130.73 | 124.03 | 112.08 | 128.49 |
| hsa-miR-1304-5p | MIMAT0005892 | 67.90 | 64.48 | 93.93 | 95.04 |
| hsa-miR-1305 | MIMAT0005893 | 127.16 | 113.37 | 84.08 | 106.46 |
| hsa-miR-1306-3p | MIMAT0005950 | 84.02 | 78.02 | 110.74 | 122.94 |
| hsa-miR-1307-3p | MIMAT0005951 | 84.09 | 98.50 | 120.45 | 87.36 |
| hsa-miR-1308 | MIMAT0005947 | 122.50 | 119.68 | 100.34 | 112.19 |
| hsa-miR-130a-3p | MIMAT0000425 | 175.16 | 213.42 | 115.12 | 111.32 |
| hsa-miR-130a-5p | MIMAT0004593 | 101.58 | 74.52 | 89.42 | 86.31 |
| hsa-miR-130b-3p | MIMAT0000691 | 121.07 | 141.58 | 120.69 | 86.88 |
| hsa-miR-130b-5p | MIMAT0004680 | 115.73 | 115.22 | 51.01 | 66.79 |
| hsa-miR-1321 | MIMAT0005952 | 51.50 | 68.29 | 86.22 | 89.75 |
| hsa-miR-1322 | MIMAT0005953 | 81.08 | 76.05 | 88.85 | 76.16 |
| hsa-miR-1323 | MIMAT0005795 | 79.22 | 66.83 | 113.34 | 125.66 |
| hsa-miR-132-3p | MIMAT0000426 | 138.93 | 154.68 | 84.13 | 97.21 |
| hsa-miR-1324 | MIMAT0005956 | 120.17 | 134.11 | 117.88 | 115.53 |
| hsa-miR-132-5p | MIMAT0004594 | 75.49 | 89.88 | 105.94 | 116.14 |
| hsa-miR-133a | MIMAT0000427 | 99.46 | 112.98 | 79.75 | 69.91 |
| hsa-miR-133b | MIMAT0000770 | 142.29 | 185.05 | 118.31 | 104.55 |
| hsa-miR-134 | MIMAT0000447 | 91.39 | 97.33 | 102.40 | 93.98 |
| hsa-miR-135a-3p | MIMAT0004595 | 124.92 | 128.04 | 96.87 | 84.85 |
| hsa-miR-135a-5p | MIMAT0000428 | 84.81 | 71.44 | 46.70 | 38.71 |
| hsa-miR-135b-3p | MIMAT0004698 | 102.13 | 103.45 | 96.49 | 112.19 |
| hsa-miR-135b-5p | MIMAT0000758 | 69.21 | 80.66 | 90.85 | 77.25 |
| hsa-miR-136-3p | MIMAT0004606 | 109.04 | 97.56 | 74.61 | 62.83 |
| hsa-miR-136-5p | MIMAT0000448 | 109.99 | 122.90 | 97.74 | 97.25 |
| hsa-miR-137 | MIMAT0000429 | 116.59 | 99.52 | 80.64 | 64.36 |
| hsa-miR-138-1-3p | MIMAT0004607 | 95.41 | 99.17 | 111.75 | 103.93 |
| hsa-miR-138-2-3p | MIMAT0004596 | 100.39 | 84.97 | 93.49 | 108.37 |
| hsa-miR-138-5p | MIMAT0000430 | 75.53 | 66.62 | 76.18 | 88.27 |
| hsa-miR-139-3p | MIMAT0004552 | 78.33 | 86.48 | 124.22 | 117.39 |
| hsa-miR-139-5p | MIMAT0000250 | 68.75 | 29.95 | 77.76 | 46.24 |
| hsa-miR-140-3p | MIMAT0004597 | 161.52 | 168.23 | 90.07 | 112.61 |
| hsa-miR-140-5p | MIMAT0000431 | 106.14 | 122.03 | 93.72 | 79.92 |
| hsa-miR-141-3p | MIMAT0000432 | 96.55 | 97.06 | 99.61 | 114.49 |
| hsa-miR-141-5p | MIMAT0004598 | 87.00 | 86.32 | 60.12 | 57.76 |
| hsa-miR-142-3p | MIMAT0000434 | 135.31 | 126.32 | 88.46 | 84.83 |
| hsa-miR-142-5p | MIMAT0000433 | 114.35 | 119.68 | 93.92 | 83.06 |
| hsa-miR-143-3p | MIMAT0000435 | 97.71 | 121.47 | 83.17 | 103.36 |
| hsa-miR-143-5p | MIMAT0004599 | 123.05 | 106.37 | 80.10 | 89.82 |
| hsa-miR-144-3p | MIMAT0000436 | 161.18 | 167.61 | 82.85 | 83.90 |
| hsa-miR-144-5p | MIMAT0004600 | 112.14 | 121.62 | 130.25 | 112.25 |
| hsa-miR-145-3p | MIMAT0004601 | 53.83 | 42.58 | 110.06 | 106.57 |
| hsa-miR-145-5p | MIMAT0000437 | 112.60 | 116.38 | 121.53 | 135.30 |
| hsa-miR-1468 | MIMAT0006789 | 73.03 | 65.59 | 84.08 | 100.25 |
| hsa-miR-1469 | MIMAT0007347 | 93.26 | 101.48 | 104.82 | 103.18 |
| hsa-miR-146a-3p | MIMAT0004608 | 116.68 | 102.55 | 71.67 | 87.84 |
| hsa-miR-146a-5p | MIMAT0000449 | 149.05 | 142.62 | 98.29 | 118.51 |
| hsa-miR-146b-3p | MIMAT0004766 | 103.50 | 113.79 | 125.67 | 112.21 |
| hsa-miR-146b-5p | MIMAT0002809 | 107.18 | 92.81 | 77.26 | 94.72 |
| hsa-miR-1470 | MIMAT0007348 | 71.76 | 76.05 | 117.90 | 123.76 |
| hsa-miR-1471 | MIMAT0007349 | 81.18 | 89.48 | 81.94 | 83.54 |
| hsa-miR-147a | MIMAT0000251 | 116.76 | 96.30 | 102.04 | 96.89 |
| hsa-miR-147b | MIMAT0004928 | 41.11 | 57.15 | 107.15 | 104.58 |
| hsa-miR-148a-3p | MIMAT0000243 | 109.32 | 122.35 | 94.43 | 103.16 |
| hsa-miR-148a-5p | MIMAT0004549 | 109.66 | 84.33 | 75.58 | 80.63 |
| hsa-miR-148b-3p | MIMAT0000759 | 123.55 | 90.26 | 125.26 | 126.32 |
| hsa-miR-148b-5p | MIMAT0004699 | 89.47 | 96.02 | 63.22 | 53.95 |
| hsa-miR-149-3p | MIMAT0004609 | 88.79 | 126.88 | 95.20 | 79.10 |
| hsa-miR-149-5p | MIMAT0000450 | 97.27 | 103.45 | 98.92 | 105.58 |
| hsa-miR-150-3p | MIMAT0004610 | 133.80 | 127.73 | 126.67 | 113.99 |
| hsa-miR-150-5p | MIMAT0000451 | 97.47 | 106.53 | 104.04 | 95.51 |
| hsa-miR-151a-3p | MIMAT0000757 | 85.74 | 63.76 | 113.91 | 123.13 |
| hsa-miR-151a-5p | MIMAT0004697 | 153.49 | 146.50 | 133.77 | 120.32 |
| hsa-miR-152 | MIMAT0000438 | 177.78 | 149.43 | 82.79 | 108.37 |
| hsa-miR-153 | MIMAT0000439 | 57.63 | 39.04 | 104.80 | 118.78 |
| hsa-miR-1537 | MIMAT0007399 | 135.14 | 123.68 | 140.11 | 145.34 |
| hsa-miR-1538 | MIMAT0007400 | 90.29 | 75.90 | 112.08 | 111.85 |
| hsa-miR-1539 | MIMAT0007401 | 194.06 | 154.64 | 114.88 | 115.90 |
| hsa-miR-154-3p | MIMAT0000453 | 113.95 | 74.32 | 84.36 | 87.13 |
| hsa-miR-154-5p | MIMAT0000452 | 84.17 | 100.90 | 89.06 | 87.46 |
| hsa-miR-155-3p | MIMAT0004658 | 128.61 | 139.04 | 104.23 | 111.07 |
| hsa-miR-155-5p | MIMAT0000646 | 148.36 | 130.95 | 84.38 | 115.16 |
| hsa-miR-15a-3p | MIMAT0004488 | 110.85 | 116.18 | 77.92 | 63.41 |
| hsa-miR-15a-5p | MIMAT0000068 | 104.58 | 85.91 | 73.92 | 87.59 |
| hsa-miR-15b-3p | MIMAT0004586 | 177.07 | 165.16 | 129.29 | 135.82 |
| hsa-miR-15b-5p | MIMAT0000417 | 69.16 | 88.37 | 86.77 | 82.99 |
| hsa-miR-16-1-3p | MIMAT0004489 | 116.50 | 89.88 | 101.95 | 93.30 |
| hsa-miR-16-2-3p | MIMAT0004518 | 80.60 | 67.44 | 85.79 | 76.46 |
| hsa-miR-16-5p | MIMAT0000069 | 68.47 | 70.77 | 75.07 | 90.15 |
| hsa-miR-17-3p | MIMAT0000071 | 73.85 | 80.73 | 109.66 | 103.18 |
| hsa-miR-17-5p | MIMAT0000070 | 95.94 | 87.30 | 85.46 | 102.44 |
| hsa-miR-181a-2-3p | MIMAT0004558 | 176.77 | 212.80 | 151.87 | 143.32 |
| hsa-miR-181a-3p | MIMAT0000270 | 68.79 | 74.76 | 98.54 | 105.59 |
| hsa-miR-181a-5p | MIMAT0000256 | 114.26 | 113.40 | 82.66 | 84.47 |
| hsa-miR-181b-5p | MIMAT0000257 | 119.01 | 94.44 | 108.04 | 89.75 |
| hsa-miR-181c-3p | MIMAT0004559 | 124.58 | 106.81 | 99.90 | 95.48 |
| hsa-miR-181c-5p | MIMAT0000258 | 95.95 | 91.75 | 74.66 | 85.13 |
| hsa-miR-181d | MIMAT0002821 | 173.03 | 156.59 | 83.97 | 92.51 |
| hsa-miR-182-3p | MIMAT0000260 | 75.49 | 76.05 | 82.02 | 93.93 |
| hsa-miR-1825 | MIMAT0006765 | 174.60 | 160.38 | 94.82 | 89.07 |
| hsa-miR-182-5p | MIMAT0000259 | 92.30 | 106.34 | 117.84 | 108.36 |
| hsa-miR-1826 | MIMAT0006766 | 93.40 | 102.55 | 124.30 | 111.23 |
| hsa-miR-1827 | MIMAT0006767 | 76.05 | 78.02 | 92.87 | 102.87 |
| hsa-miR-183-3p | MIMAT0004560 | 116.30 | 86.44 | 103.67 | 74.59 |
| hsa-miR-183-5p | MIMAT0000261 | 106.08 | 90.86 | 112.04 | 93.19 |
| hsa-miR-184 | MIMAT0000454 | 85.71 | 72.95 | 110.41 | 102.36 |
| hsa-miR-185-3p | MIMAT0004611 | 43.80 | 45.32 | 87.14 | 101.55 |
| hsa-miR-185-5p | MIMAT0000455 | 143.83 | 131.44 | 94.56 | 92.57 |
| hsa-miR-186-3p | MIMAT0004612 | 140.49 | 130.73 | 103.96 | 120.30 |
| hsa-miR-186-5p | MIMAT0000456 | 128.58 | 107.84 | 115.34 | 86.50 |
| hsa-miR-187-3p | MIMAT0000262 | 96.93 | 90.32 | 79.00 | 74.12 |
| hsa-miR-187-5p | MIMAT0004561 | 91.43 | 71.14 | 114.11 | 118.98 |
| hsa-miR-188-3p | MIMAT0004613 | 69.78 | 74.84 | 72.98 | 69.06 |
| hsa-miR-188-5p | MIMAT0000457 | 130.25 | 129.52 | 83.63 | 79.40 |
| hsa-miR-18a-3p | MIMAT0002891 | 79.51 | 94.85 | 101.41 | 110.02 |
| hsa-miR-18a-5p | MIMAT0000072 | 100.26 | 126.11 | 142.80 | 163.49 |
| hsa-miR-18b-3p | MIMAT0004751 | 90.40 | 116.00 | 129.86 | 127.57 |
| hsa-miR-18b-5p | MIMAT0001412 | 94.45 | 108.74 | 122.87 | 106.26 |
| hsa-miR-1908 | MIMAT0007881 | 78.33 | 61.62 | 125.91 | 124.79 |
| hsa-miR-1909-3p | MIMAT0007883 | 82.01 | 82.20 | 115.62 | 116.14 |
| hsa-miR-1909-5p | MIMAT0007882 | 143.40 | 132.64 | 110.58 | 92.84 |
| hsa-miR-190a | MIMAT0000458 | 66.13 | 75.39 | 85.77 | 90.24 |
| hsa-miR-190b | MIMAT0004929 | 78.15 | 63.78 | 97.83 | 98.47 |
| hsa-miR-1910 | MIMAT0007884 | 138.86 | 123.68 | 109.92 | 110.43 |
| hsa-miR-1911-3p | MIMAT0007886 | 84.13 | 92.67 | 85.01 | 87.34 |
| hsa-miR-1911-5p | MIMAT0007885 | 111.36 | 127.21 | 94.56 | 97.06 |
| hsa-miR-1912 | MIMAT0007887 | 71.29 | 81.37 | 91.78 | 99.77 |
| hsa-miR-1913 | MIMAT0007888 | 88.32 | 105.52 | 120.42 | 104.02 |
| hsa-miR-191-3p | MIMAT0001618 | 122.77 | 149.72 | 152.22 | 151.35 |
| hsa-miR-1914-3p | MIMAT0007890 | 110.90 | 114.46 | 120.75 | 121.22 |
| hsa-miR-1914-5p | MIMAT0007889 | 125.63 | 126.14 | 106.16 | 110.82 |
| hsa-miR-1915-3p | MIMAT0007892 | 128.53 | 108.52 | 102.97 | 97.08 |
| hsa-miR-1915-5p | MIMAT0007891 | 81.37 | 77.55 | 95.96 | 67.87 |
| hsa-miR-191-5p | MIMAT0000440 | 94.13 | 84.13 | 131.97 | 96.76 |
| hsa-miR-192-3p | MIMAT0004543 | 131.07 | 142.51 | 103.92 | 89.56 |
| hsa-miR-192-5p | MIMAT0000222 | 93.88 | 102.09 | 91.85 | 86.35 |
| hsa-miR-193a-3p | MIMAT0000459 | 102.91 | 90.56 | 112.33 | 72.78 |
| hsa-miR-193a-5p | MIMAT0004614 | 114.60 | 115.93 | 106.51 | 105.74 |
| hsa-miR-193b-3p | MIMAT0002819 | 81.18 | 77.31 | 115.74 | 85.93 |
| hsa-miR-193b-5p | MIMAT0004767 | 166.57 | 199.38 | 141.69 | 145.03 |
| hsa-miR-194-3p | MIMAT0004671 | 97.86 | 91.42 | 110.49 | 97.74 |
| hsa-miR-194-5p | MIMAT0000460 | 172.95 | 174.42 | 60.48 | 78.78 |
| hsa-miR-195-3p | MIMAT0004615 | 120.57 | 142.81 | 102.35 | 105.16 |
| hsa-miR-195-5p | MIMAT0000461 | 59.59 | 92.58 | 78.07 | 75.30 |
| hsa-miR-196a-3p | MIMAT0004562 | 114.75 | 108.52 | 122.90 | 120.98 |
| hsa-miR-196a-5p | MIMAT0000226 | 92.57 | 92.83 | 78.04 | 68.80 |
| hsa-miR-196b-3p | MIMAT0009201 | 115.52 | 104.36 | 124.30 | 91.66 |
| hsa-miR-196b-5p | MIMAT0001080 | 89.02 | 74.44 | 72.24 | 54.49 |
| hsa-miR-1972 | MIMAT0009447 | 134.19 | 146.58 | 109.52 | 117.99 |
| hsa-miR-1972 | MIMAT0009447 | 117.74 | 87.84 | 87.12 | 102.80 |
| hsa-miR-1973 | MIMAT0009448 | 112.50 | 89.12 | 99.28 | 89.66 |
| hsa-miR-197-3p | MIMAT0000227 | 54.84 | 55.26 | 92.84 | 85.51 |
| hsa-miR-1974 | MIMAT0009449 | 119.67 | 101.48 | 105.57 | 112.62 |
| hsa-miR-1975 | MIMAT0009450 | 106.24 | 91.42 | 69.49 | 69.18 |
| hsa-miR-1976 | MIMAT0009451 | 64.48 | 73.64 | 93.93 | 79.20 |
| hsa-miR-1977 | MIMAT0009452 | 82.93 | 90.38 | 85.36 | 110.28 |
| hsa-miR-1978 | MIMAT0009453 | 75.49 | 77.59 | 121.89 | 104.09 |
| hsa-miR-1979 | MIMAT0009454 | 141.23 | 137.14 | 96.26 | 124.51 |
| hsa-miR-198 | MIMAT0000228 | 117.20 | 115.10 | 106.39 | 93.73 |
| hsa-miR-199a-3p | MIMAT0000232 | 123.08 | 121.94 | 99.48 | 94.52 |
| hsa-miR-199a-5p | MIMAT0000231 | 80.32 | 72.95 | 108.55 | 90.87 |
| hsa-miR-199b-5p | MIMAT0000263 | 90.49 | 118.78 | 86.22 | 90.23 |
| hsa-miR-19a-3p | MIMAT0000073 | 107.81 | 113.91 | 117.27 | 115.72 |
| hsa-miR-19a-5p | MIMAT0004490 | 50.23 | 49.19 | 124.73 | 109.32 |
| hsa-miR-19b-1-5p | MIMAT0004491 | 36.55 | 45.32 | 90.42 | 76.21 |
| hsa-miR-19b-2-5p | MIMAT0004492 | 37.00 | 35.70 | 103.66 | 92.57 |
| hsa-miR-19b-3p | MIMAT0000074 | 154.62 | 164.41 | 94.91 | 122.60 |
| hsa-miR-200a-3p | MIMAT0000682 | 82.95 | 80.66 | 110.49 | 92.03 |
| hsa-miR-200a-5p | MIMAT0001620 | 82.47 | 98.23 | 100.73 | 91.69 |
| hsa-miR-200b-3p | MIMAT0000318 | 50.55 | 52.33 | 57.75 | 66.35 |
| hsa-miR-200b-5p | MIMAT0004571 | 75.58 | 89.15 | 119.57 | 113.45 |
| hsa-miR-200c-3p | MIMAT0000617 | 82.47 | 83.97 | 61.63 | 63.38 |
| hsa-miR-200c-5p | MIMAT0004657 | 108.11 | 120.61 | 102.52 | 107.89 |
| hsa-miR-202-3p | MIMAT0002811 | 91.09 | 85.98 | 135.72 | 103.64 |
| hsa-miR-202-5p | MIMAT0002810 | 97.50 | 90.18 | 106.19 | 93.41 |
| hsa-miR-203a | MIMAT0000264 | 106.73 | 110.55 | 110.04 | 101.95 |
| hsa-miR-204-5p | MIMAT0000265 | 107.48 | 101.94 | 117.07 | 120.93 |
| hsa-miR-2052 | MIMAT0009977 | 40.43 | 35.71 | 100.73 | 92.51 |
| hsa-miR-2053 | MIMAT0009978 | 53.83 | 45.30 | 74.24 | 92.61 |
| hsa-miR-205-3p | MIMAT0009197 | 40.99 | 53.26 | 83.90 | 86.81 |
| hsa-miR-2054 | MIMAT0009979 | 102.96 | 104.72 | 106.68 | 98.26 |
| hsa-miR-205-5p | MIMAT0000266 | 85.84 | 97.14 | 105.47 | 119.35 |
| hsa-miR-206 | MIMAT0000462 | 143.45 | 121.41 | 80.81 | 73.61 |
| hsa-miR-208a | MIMAT0000241 | 109.11 | 120.13 | 56.69 | 46.31 |
| hsa-miR-208b | MIMAT0004960 | 112.15 | 124.71 | 79.73 | 80.71 |
| hsa-miR-20a-3p | MIMAT0004493 | 117.03 | 108.50 | 107.64 | 84.78 |
| hsa-miR-20a-5p | MIMAT0000075 | 122.90 | 124.82 | 108.55 | 124.10 |
| hsa-miR-20b-3p | MIMAT0004752 | 111.46 | 126.50 | 120.13 | 124.59 |
| hsa-miR-20b-5p | MIMAT0001413 | 124.49 | 105.05 | 114.87 | 111.62 |
| hsa-miR-210 | MIMAT0000267 | 117.72 | 95.35 | 115.90 | 97.41 |
| hsa-miR-2110 | MIMAT0010133 | 84.81 | 74.52 | 106.51 | 84.41 |
| hsa-miR-2113 | MIMAT0009206 | 146.94 | 177.83 | 120.02 | 118.87 |
| hsa-miR-2114-3p | MIMAT0011157 | 78.42 | 79.72 | 77.84 | 82.63 |
| hsa-miR-2114-5p | MIMAT0011156 | 97.35 | 108.45 | 86.13 | 84.46 |
| hsa-miR-2115-3p | MIMAT0011159 | 67.88 | 70.54 | 85.66 | 79.24 |
| hsa-miR-2115-5p | MIMAT0011158 | 71.96 | 66.09 | 97.82 | 82.85 |
| hsa-miR-211-5p | MIMAT0000268 | 112.02 | 92.64 | 93.49 | 101.68 |
| hsa-miR-2116-3p | MIMAT0011161 | 89.02 | 77.22 | 87.87 | 79.21 |
| hsa-miR-2116-5p | MIMAT0011160 | 76.57 | 78.94 | 85.09 | 128.63 |
| hsa-miR-2117 | MIMAT0011162 | 85.49 | 92.42 | 117.80 | 112.80 |
| hsa-miR-212-3p | MIMAT0000269 | 105.69 | 107.21 | 62.78 | 81.69 |
| hsa-miR-21-3p | MIMAT0004494 | 86.67 | 83.73 | 97.39 | 89.49 |
| hsa-miR-214-3p | MIMAT0000271 | 113.07 | 138.59 | 86.42 | 93.32 |
| hsa-miR-214-5p | MIMAT0004564 | 121.57 | 78.40 | 94.27 | 79.61 |
| hsa-miR-215 | MIMAT0000272 | 90.86 | 96.00 | 107.37 | 116.87 |
| hsa-miR-21-5p | MIMAT0000076 | 110.59 | 121.46 | 112.80 | 105.38 |
| hsa-miR-216a-5p | MIMAT0000273 | 116.50 | 124.28 | 96.76 | 99.23 |
| hsa-miR-216b | MIMAT0004959 | 169.78 | 172.43 | 116.53 | 118.25 |
| hsa-miR-217 | MIMAT0000274 | 109.75 | 99.72 | 64.39 | 80.92 |
| hsa-miR-218-1-3p | MIMAT0004565 | 91.81 | 110.29 | 72.19 | 62.93 |
| hsa-miR-218-2-3p | MIMAT0004566 | 112.15 | 106.28 | 89.33 | 107.52 |
| hsa-miR-218-5p | MIMAT0000275 | 83.07 | 86.29 | 99.94 | 99.21 |
| hsa-miR-219-1-3p | MIMAT0004567 | 78.15 | 99.72 | 111.21 | 116.45 |
| hsa-miR-219-2-3p | MIMAT0004675 | 49.33 | 57.21 | 138.44 | 141.06 |
| hsa-miR-219-5p | MIMAT0000276 | 84.15 | 98.59 | 73.01 | 90.63 |
| hsa-miR-220a | MIMAT0000277 | 80.32 | 93.05 | 105.19 | 91.69 |
| hsa-miR-220b | MIMAT0004908 | 92.26 | 82.20 | 105.94 | 104.09 |
| hsa-miR-221-3p | MIMAT0000278 | 142.31 | 139.73 | 110.78 | 120.82 |
| hsa-miR-221-5p | MIMAT0004568 | 108.04 | 92.67 | 97.28 | 104.46 |
| hsa-miR-222-3p | MIMAT0000279 | 97.75 | 118.23 | 79.09 | 87.81 |
| hsa-miR-222-5p | MIMAT0004569 | 45.34 | 106.70 | 89.53 | 91.03 |
| hsa-miR-223-3p | MIMAT0000280 | 113.82 | 98.12 | 82.72 | 76.97 |
| hsa-miR-223-5p | MIMAT0004570 | 83.86 | 74.16 | 83.23 | 82.97 |
| hsa-miR-22-3p | MIMAT0000077 | 56.06 | 43.12 | 78.01 | 67.08 |
| hsa-miR-224-3p | MIMAT0009198 | 65.71 | 61.75 | 99.32 | 110.46 |
| hsa-miR-224-5p | MIMAT0000281 | 76.04 | 63.08 | 109.42 | 104.22 |
| hsa-miR-22-5p | MIMAT0004495 | 90.29 | 103.40 | 85.12 | 117.66 |
| hsa-miR-2276 | MIMAT0011775 | 89.12 | 89.44 | 98.34 | 105.48 |
| hsa-miR-2277-3p | MIMAT0011777 | 103.84 | 120.29 | 120.76 | 115.88 |
| hsa-miR-2277-5p | MIMAT0017352 | 62.31 | 46.63 | 95.20 | 82.85 |
| hsa-miR-2278 | MIMAT0011778 | 101.10 | 126.54 | 96.74 | 97.36 |
| hsa-miR-2355-3p | MIMAT0017950 | 89.56 | 97.60 | 120.80 | 114.49 |
| hsa-miR-2355-5p | MIMAT0016895 | 144.95 | 144.71 | 103.03 | 119.72 |
| hsa-miR-23a-3p | MIMAT0000078 | 123.02 | 117.53 | 73.47 | 88.85 |
| hsa-miR-23a-5p | MIMAT0004496 | 120.64 | 136.43 | 88.90 | 97.80 |
| hsa-miR-23b-3p | MIMAT0000418 | 103.77 | 112.40 | 116.79 | 98.04 |
| hsa-miR-23b-5p | MIMAT0004587 | 106.45 | 78.96 | 77.14 | 73.53 |
| hsa-miR-23c | MIMAT0018000 | 85.10 | 101.04 | 75.80 | 90.37 |
| hsa-miR-24-1-5p | MIMAT0000079 | 97.27 | 78.96 | 96.88 | 84.56 |
| hsa-miR-24-2-5p | MIMAT0004497 | 93.84 | 96.75 | 83.13 | 84.17 |
| hsa-miR-24-3p | MIMAT0000080 | 93.28 | 105.52 | 106.94 | 125.15 |
| hsa-miR-25-3p | MIMAT0000081 | 63.24 | 65.68 | 97.73 | 78.77 |
| hsa-miR-25-5p | MIMAT0004498 | 99.22 | 93.27 | 91.90 | 110.65 |
| hsa-miR-26a-1-3p | MIMAT0004499 | 93.69 | 103.54 | 103.25 | 107.42 |
| hsa-miR-26a-2-3p | MIMAT0004681 | 110.79 | 88.07 | 100.29 | 86.49 |
| hsa-miR-26a-5p | MIMAT0000082 | 113.07 | 120.86 | 99.95 | 80.42 |
| hsa-miR-26b-3p | MIMAT0004500 | 98.64 | 91.73 | 82.79 | 105.50 |
| hsa-miR-26b-5p | MIMAT0000083 | 70.46 | 91.93 | 76.84 | 105.02 |
| hsa-miR-27a-3p | MIMAT0000084 | 170.19 | 144.71 | 111.55 | 127.42 |
| hsa-miR-27a-5p | MIMAT0004501 | 112.61 | 89.93 | 128.15 | 121.73 |
| hsa-miR-27b-3p | MIMAT0000419 | 132.98 | 149.59 | 104.30 | 89.81 |
| hsa-miR-27b-5p | MIMAT0004588 | 98.79 | 112.92 | 95.12 | 104.72 |
| hsa-miR-28-3p | MIMAT0004502 | 54.99 | 26.89 | 111.63 | 94.57 |
| hsa-miR-28-5p | MIMAT0000085 | 40.74 | 34.42 | 73.17 | 72.82 |
| hsa-miR-2861 | MIMAT0013802 | 66.92 | 88.91 | 100.30 | 94.96 |
| hsa-miR-2909 | MIMAT0013863 | 78.10 | 95.31 | 103.97 | 79.61 |
| hsa-miR-296-3p | MIMAT0004679 | 100.76 | 100.24 | 95.92 | 88.42 |
| hsa-miR-296-5p | MIMAT0000690 | 75.42 | 93.65 | 95.40 | 99.61 |
| hsa-miR-297 | MIMAT0004450 | 60.75 | 60.73 | 84.53 | 79.10 |
| hsa-miR-298 | MIMAT0004901 | 84.61 | 98.50 | 108.71 | 97.57 |
| hsa-miR-299-3p | MIMAT0000687 | 128.60 | 104.32 | 99.51 | 110.03 |
| hsa-miR-299-5p | MIMAT0002890 | 96.31 | 85.59 | 94.84 | 82.38 |
| hsa-miR-29a-3p | MIMAT0000086 | 54.08 | 79.25 | 67.99 | 84.28 |
| hsa-miR-29a-5p | MIMAT0004503 | 112.31 | 125.79 | 110.65 | 129.72 |
| hsa-miR-29b-1-5p | MIMAT0004514 | 93.52 | 101.83 | 125.15 | 124.59 |
| hsa-miR-29b-2-5p | MIMAT0004515 | 74.65 | 61.98 | 108.65 | 97.69 |
| hsa-miR-29b-3p | MIMAT0000100 | 118.09 | 132.64 | 101.97 | 72.19 |
| hsa-miR-29c-3p | MIMAT0000681 | 73.93 | 91.96 | 44.61 | 55.13 |
| hsa-miR-29c-5p | MIMAT0004673 | 95.15 | 90.44 | 113.92 | 113.19 |
| hsa-miR-300 | MIMAT0004903 | 126.73 | 137.37 | 97.91 | 116.74 |
| hsa-miR-301a-3p | MIMAT0000688 | 86.15 | 103.73 | 113.09 | 93.98 |
| hsa-miR-301b | MIMAT0004958 | 200.53 | 207.16 | 116.74 | 113.85 |
| hsa-miR-302a-3p | MIMAT0000684 | 123.05 | 121.62 | 146.53 | 148.85 |
| hsa-miR-302a-5p | MIMAT0000683 | 120.90 | 72.56 | 107.17 | 98.25 |
| hsa-miR-302b-3p | MIMAT0000715 | 123.02 | 126.75 | 129.86 | 126.30 |
| hsa-miR-302b-5p | MIMAT0000714 | 88.64 | 86.87 | 96.20 | 93.50 |
| hsa-miR-302c-3p | MIMAT0000717 | 185.42 | 233.52 | 130.29 | 123.47 |
| hsa-miR-302c-5p | MIMAT0000716 | 116.50 | 106.78 | 112.77 | 109.16 |
| hsa-miR-302d-3p | MIMAT0000718 | 89.25 | 89.85 | 134.07 | 144.28 |
| hsa-miR-302d-5p | MIMAT0004685 | 92.67 | 115.93 | 71.08 | 76.21 |
| hsa-miR-302e | MIMAT0005931 | 146.11 | 139.37 | 143.10 | 133.24 |
| hsa-miR-302f | MIMAT0005932 | 118.05 | 99.53 | 82.11 | 105.64 |
| hsa-miR-3065-3p | MIMAT0015378 | 90.69 | 78.54 | 99.51 | 91.43 |
| hsa-miR-3065-5p | MIMAT0015066 | 128.50 | 114.95 | 108.00 | 122.54 |
| hsa-miR-3074-3p | MIMAT0015027 | 42.83 | 52.05 | 85.07 | 65.69 |
| hsa-miR-30a-3p | MIMAT0000088 | 129.95 | 118.27 | 69.39 | 81.23 |
| hsa-miR-30a-5p | MIMAT0000087 | 94.27 | 78.71 | 104.53 | 98.77 |
| hsa-miR-30b-3p | MIMAT0004589 | 118.82 | 128.78 | 102.76 | 80.18 |
| hsa-miR-30b-5p | MIMAT0000420 | 118.05 | 104.07 | 107.06 | 112.21 |
| hsa-miR-30c-1-3p | MIMAT0004674 | 102.06 | 104.65 | 134.62 | 121.16 |
| hsa-miR-30c-2-3p | MIMAT0004550 | 121.07 | 107.99 | 110.42 | 127.52 |
| hsa-miR-30c-5p | MIMAT0000244 | 62.44 | 55.93 | 81.77 | 93.80 |
| hsa-miR-30d-3p | MIMAT0004551 | 102.91 | 120.86 | 86.13 | 83.06 |
| hsa-miR-30d-5p | MIMAT0000245 | 69.99 | 78.45 | 119.30 | 109.34 |
| hsa-miR-30e-3p | MIMAT0000693 | 104.62 | 27.13 | 78.88 | 44.10 |
| hsa-miR-30e-5p | MIMAT0000692 | 101.04 | 89.22 | 119.54 | 109.60 |
| hsa-miR-3115 | MIMAT0014977 | 81.25 | 117.34 | 68.31 | 90.93 |
| hsa-miR-3116 | MIMAT0014978 | 84.89 | 82.42 | 109.60 | 137.02 |
| hsa-miR-3117-3p | MIMAT0014979 | 85.77 | 97.58 | 84.64 | 83.11 |
| hsa-miR-3118 | MIMAT0014980 | 108.26 | 69.40 | 112.27 | 101.62 |
| hsa-miR-3119 | MIMAT0014981 | 66.25 | 61.63 | 86.07 | 104.30 |
| hsa-miR-3120-3p | MIMAT0014982 | 97.91 | 109.31 | 122.03 | 114.28 |
| hsa-miR-3121-3p | MIMAT0014983 | 125.85 | 115.49 | 71.29 | 76.78 |
| hsa-miR-3122 | MIMAT0014984 | 76.87 | 107.66 | 97.28 | 96.58 |
| hsa-miR-3123 | MIMAT0014985 | 175.93 | 142.40 | 127.65 | 101.27 |
| hsa-miR-3124-5p | MIMAT0014986 | 92.68 | 101.94 | 120.80 | 108.06 |
| hsa-miR-3125 | MIMAT0014988 | 100.35 | 103.93 | 97.84 | 95.78 |
| hsa-miR-3126-3p | MIMAT0015377 | 89.56 | 107.36 | 100.00 | 73.74 |
| hsa-miR-3126-5p | MIMAT0014989 | 121.87 | 115.42 | 101.78 | 105.09 |
| hsa-miR-3127-5p | MIMAT0014990 | 109.22 | 112.44 | 124.42 | 104.76 |
| hsa-miR-3128 | MIMAT0014991 | 65.20 | 78.81 | 107.64 | 121.30 |
| hsa-miR-3129-5p | MIMAT0014992 | 117.45 | 127.33 | 82.32 | 79.95 |
| hsa-miR-3130-3p | MIMAT0014994 | 79.22 | 72.37 | 100.31 | 107.81 |
| hsa-miR-3130-5p | MIMAT0014995 | 80.28 | 89.43 | 95.97 | 93.77 |
| hsa-miR-3131 | MIMAT0014996 | 83.26 | 124.16 | 115.03 | 102.51 |
| hsa-miR-3132 | MIMAT0014997 | 93.66 | 90.84 | 95.07 | 101.72 |
| hsa-miR-3133 | MIMAT0014998 | 60.02 | 68.42 | 75.39 | 78.60 |
| hsa-miR-3134 | MIMAT0015000 | 86.91 | 99.64 | 88.12 | 110.22 |
| hsa-miR-3135a | MIMAT0015001 | 84.81 | 112.63 | 85.66 | 101.23 |
| hsa-miR-3136-5p | MIMAT0015003 | 78.12 | 82.82 | 97.29 | 119.72 |
| hsa-miR-3137 | MIMAT0015005 | 95.35 | 78.96 | 140.39 | 138.44 |
| hsa-miR-3138 | MIMAT0015006 | 70.84 | 65.69 | 106.19 | 114.43 |
| hsa-miR-3139 | MIMAT0015007 | 84.11 | 60.73 | 65.33 | 61.94 |
| hsa-miR-31-3p | MIMAT0004504 | 110.60 | 106.77 | 112.33 | 139.14 |
| hsa-miR-3140-3p | MIMAT0015008 | 85.77 | 99.88 | 93.82 | 111.38 |
| hsa-miR-3141 | MIMAT0015010 | 92.30 | 88.04 | 99.57 | 116.86 |
| hsa-miR-3142 | MIMAT0015011 | 101.98 | 77.22 | 124.81 | 83.66 |
| hsa-miR-3143 | MIMAT0015012 | 130.06 | 146.40 | 114.93 | 126.83 |
| hsa-miR-3144-3p | MIMAT0015015 | 125.38 | 120.28 | 84.98 | 118.38 |
| hsa-miR-3144-5p | MIMAT0015014 | 73.21 | 65.07 | 76.00 | 76.95 |
| hsa-miR-3145-3p | MIMAT0015016 | 79.06 | 105.20 | 82.01 | 95.87 |
| hsa-miR-3146 | MIMAT0015018 | 98.13 | 121.46 | 84.00 | 87.14 |
| hsa-miR-3147 | MIMAT0015019 | 77.69 | 82.76 | 121.40 | 115.60 |
| hsa-miR-3148 | MIMAT0015021 | 108.63 | 126.76 | 133.10 | 128.28 |
| hsa-miR-3149 | MIMAT0015022 | 118.82 | 107.26 | 78.23 | 88.35 |
| hsa-miR-3150a-3p | MIMAT0015023 | 59.00 | 75.14 | 111.55 | 118.10 |
| hsa-miR-3150b-3p | MIMAT0018194 | 90.38 | 112.71 | 99.24 | 123.77 |
| hsa-miR-3151 | MIMAT0015024 | 86.15 | 75.83 | 103.59 | 92.02 |
| hsa-miR-3152-3p | MIMAT0015025 | 121.87 | 104.72 | 100.62 | 112.83 |
| hsa-miR-3153 | MIMAT0015026 | 164.33 | 134.47 | 135.73 | 134.87 |
| hsa-miR-3154 | MIMAT0015028 | 87.59 | 88.70 | 83.14 | 87.96 |
| hsa-miR-3155a | MIMAT0015029 | 150.31 | 129.05 | 114.93 | 120.93 |
| hsa-miR-3156-5p | MIMAT0015030 | 104.81 | 109.88 | 102.28 | 89.45 |
| hsa-miR-3157-5p | MIMAT0015031 | 82.20 | 69.57 | 82.66 | 85.28 |
| hsa-miR-3158-3p | MIMAT0015032 | 106.21 | 102.17 | 98.92 | 114.52 |
| hsa-miR-3159 | MIMAT0015033 | 164.05 | 180.89 | 114.39 | 130.66 |
| hsa-miR-31-5p | MIMAT0000089 | 84.58 | 86.58 | 133.23 | 137.00 |
| hsa-miR-3160-3p | MIMAT0015034 | 132.73 | 116.71 | 98.24 | 93.50 |
| hsa-miR-3161 | MIMAT0015035 | 84.74 | 85.68 | 90.08 | 92.22 |
| hsa-miR-3162-5p | MIMAT0015036 | 86.91 | 106.34 | 116.01 | 87.77 |
| hsa-miR-3163 | MIMAT0015037 | 97.53 | 124.25 | 125.86 | 124.01 |
| hsa-miR-3164 | MIMAT0015038 | 101.11 | 117.37 | 108.67 | 104.62 |
| hsa-miR-3165 | MIMAT0015039 | 42.63 | 46.61 | 61.34 | 75.15 |
| hsa-miR-3166 | MIMAT0015040 | 75.38 | 82.09 | 93.08 | 76.78 |
| hsa-miR-3167 | MIMAT0015042 | 91.78 | 108.28 | 84.29 | 105.46 |
| hsa-miR-3168 | MIMAT0015043 | 111.67 | 84.98 | 100.56 | 90.23 |
| hsa-miR-3169 | MIMAT0015044 | 56.96 | 52.87 | 119.13 | 116.48 |
| hsa-miR-3170 | MIMAT0015045 | 133.06 | 143.82 | 139.03 | 151.18 |
| hsa-miR-3171 | MIMAT0015046 | 85.29 | 92.31 | 93.26 | 101.67 |
| hsa-miR-3173-3p | MIMAT0015048 | 73.28 | 81.16 | 101.82 | 102.79 |
| hsa-miR-3174 | MIMAT0015051 | 60.53 | 76.59 | 92.20 | 98.05 |
| hsa-miR-3175 | MIMAT0015052 | 81.51 | 95.31 | 112.50 | 94.60 |
| hsa-miR-3176 | MIMAT0015053 | 125.63 | 97.58 | 126.67 | 111.61 |
| hsa-miR-3177-3p | MIMAT0015054 | 28.82 | 37.96 | 92.00 | 82.32 |
| hsa-miR-3178 | MIMAT0015055 | 95.97 | 97.61 | 119.44 | 119.88 |
| hsa-miR-3179 | MIMAT0015056 | 81.94 | 88.40 | 83.28 | 102.68 |
| hsa-miR-3180 | MIMAT0018178 | 73.63 | 113.65 | 105.04 | 116.69 |
| hsa-miR-3180-3p | MIMAT0015058 | 91.12 | 72.66 | 121.33 | 137.02 |
| hsa-miR-3180-5p | MIMAT0015057 | 88.36 | 103.54 | 101.78 | 105.09 |
| hsa-miR-3181 | MIMAT0015061 | 54.91 | 60.53 | 118.18 | 78.40 |
| hsa-miR-3182 | MIMAT0015062 | 134.06 | 152.53 | 117.47 | 102.36 |
| hsa-miR-3183 | MIMAT0015063 | 113.73 | 109.08 | 122.79 | 117.15 |
| hsa-miR-3184-5p | MIMAT0015064 | 65.14 | 53.57 | 103.50 | 95.82 |
| hsa-miR-3185 | MIMAT0015065 | 138.13 | 153.06 | 104.45 | 120.13 |
| hsa-miR-3186-3p | MIMAT0015068 | 59.19 | 41.21 | 85.60 | 91.97 |
| hsa-miR-3186-5p | MIMAT0015067 | 72.11 | 99.17 | 85.09 | 77.57 |
| hsa-miR-3187-3p | MIMAT0015069 | 93.46 | 124.71 | 108.53 | 131.65 |
| hsa-miR-3188 | MIMAT0015070 | 131.77 | 107.26 | 106.19 | 90.30 |
| hsa-miR-3189-3p | MIMAT0015071 | 88.45 | 64.50 | 95.55 | 84.50 |
| hsa-miR-3190 | MIMAT0015073 | 123.81 | 131.49 | 125.29 | 108.38 |
| hsa-miR-3191-3p | MIMAT0015075 | 95.16 | 88.36 | 112.97 | 107.16 |
| hsa-miR-3192 | MIMAT0015076 | 92.30 | 71.48 | 70.70 | 61.29 |
| hsa-miR-3193 | MIMAT0015077 | 74.69 | 98.79 | 87.39 | 113.64 |
| hsa-miR-3194-5p | MIMAT0015078 | 127.73 | 108.45 | 81.87 | 64.62 |
| hsa-miR-3195 | MIMAT0015079 | 111.97 | 108.00 | 95.89 | 82.13 |
| hsa-miR-3196 | MIMAT0015080 | 95.16 | 88.36 | 98.29 | 114.86 |
| hsa-miR-3197 | MIMAT0015082 | 94.56 | 108.13 | 138.78 | 127.39 |
| hsa-miR-3198 | MIMAT0015083 | 151.77 | 180.89 | 83.60 | 71.91 |
| hsa-miR-3199 | MIMAT0015084 | 121.87 | 116.46 | 94.50 | 89.87 |
| hsa-miR-3200-3p | MIMAT0015085 | 89.95 | 115.43 | 82.88 | 93.80 |
| hsa-miR-3200-5p | MIMAT0017392 | 83.03 | 74.48 | 108.48 | 102.55 |
| hsa-miR-3201 | MIMAT0015086 | 117.60 | 123.63 | 113.33 | 111.27 |
| hsa-miR-3202 | MIMAT0015089 | 125.70 | 99.88 | 98.58 | 97.85 |
| hsa-miR-320a | MIMAT0000510 | 70.62 | 68.41 | 98.12 | 85.54 |
| hsa-miR-320b | MIMAT0005792 | 120.48 | 111.87 | 127.59 | 135.09 |
| hsa-miR-320c | MIMAT0005793 | 86.83 | 98.74 | 97.88 | 123.20 |
| hsa-miR-320d | MIMAT0006764 | 112.50 | 122.45 | 92.20 | 78.84 |
| hsa-miR-320e | MIMAT0015072 | 118.23 | 114.14 | 105.12 | 115.34 |
| hsa-miR-323a-3p | MIMAT0000755 | 73.44 | 86.87 | 69.63 | 81.51 |
| hsa-miR-323a-5p | MIMAT0004696 | 85.38 | 91.52 | 92.99 | 90.38 |
| hsa-miR-323b-3p | MIMAT0015050 | 69.58 | 52.18 | 79.91 | 74.92 |
| hsa-miR-323b-5p | MIMAT0001630 | 107.40 | 90.18 | 111.59 | 97.69 |
| hsa-miR-32-3p | MIMAT0004505 | 112.02 | 128.25 | 112.75 | 123.17 |
| hsa-miR-324-3p | MIMAT0000762 | 96.55 | 117.16 | 101.63 | 109.47 |
| hsa-miR-324-5p | MIMAT0000761 | 74.34 | 82.25 | 103.99 | 83.06 |
| hsa-miR-325 | MIMAT0000771 | 94.39 | 84.31 | 109.45 | 101.47 |
| hsa-miR-32-5p | MIMAT0000090 | 86.67 | 74.03 | 99.25 | 88.63 |
| hsa-miR-326 | MIMAT0000756 | 76.41 | 97.04 | 78.06 | 61.68 |
| hsa-miR-328 | MIMAT0000752 | 183.06 | 180.43 | 84.95 | 103.13 |
| hsa-miR-329 | MIMAT0001629 | 76.53 | 71.45 | 82.37 | 98.82 |
| hsa-miR-330-3p | MIMAT0000751 | 140.42 | 154.18 | 105.68 | 113.43 |
| hsa-miR-330-5p | MIMAT0004693 | 102.21 | 102.13 | 141.60 | 121.45 |
| hsa-miR-331-3p | MIMAT0000760 | 112.24 | 130.82 | 125.31 | 99.03 |
| hsa-miR-331-5p | MIMAT0004700 | 107.42 | 110.88 | 94.68 | 78.28 |
| hsa-miR-335-3p | MIMAT0004703 | 81.56 | 85.62 | 87.24 | 78.35 |
| hsa-miR-335-5p | MIMAT0000765 | 84.55 | 86.47 | 116.49 | 114.82 |
| hsa-miR-337-3p | MIMAT0000754 | 91.12 | 101.94 | 113.33 | 110.74 |
| hsa-miR-337-5p | MIMAT0004695 | 127.73 | 109.53 | 102.13 | 111.81 |
| hsa-miR-338-3p | MIMAT0000763 | 110.65 | 124.17 | 77.06 | 105.17 |
| hsa-miR-338-5p | MIMAT0004701 | 98.24 | 83.48 | 87.19 | 82.18 |
| hsa-miR-339-3p | MIMAT0004702 | 121.51 | 95.78 | 73.14 | 78.33 |
| hsa-miR-339-5p | MIMAT0000764 | 122.90 | 128.71 | 79.13 | 85.54 |
| hsa-miR-33a-3p | MIMAT0004506 | 72.94 | 78.40 | 80.46 | 70.81 |
| hsa-miR-33a-5p | MIMAT0000091 | 105.87 | 110.97 | 65.48 | 65.39 |
| hsa-miR-33b-3p | MIMAT0004811 | 98.24 | 111.45 | 100.67 | 100.76 |
| hsa-miR-33b-5p | MIMAT0003301 | 75.23 | 113.91 | 76.14 | 69.34 |
| hsa-miR-340-3p | MIMAT0000750 | 111.36 | 121.39 | 103.66 | 113.70 |
| hsa-miR-340-5p | MIMAT0004692 | 73.74 | 88.44 | 141.77 | 110.64 |
| hsa-miR-342-3p | MIMAT0000753 | 118.09 | 100.43 | 96.21 | 91.04 |
| hsa-miR-342-5p | MIMAT0004694 | 98.70 | 79.78 | 106.67 | 90.93 |
| hsa-miR-345-5p | MIMAT0000772 | 108.92 | 87.21 | 81.18 | 82.90 |
| hsa-miR-346 | MIMAT0000773 | 115.52 | 110.67 | 102.48 | 116.48 |
| hsa-miR-34a-3p | MIMAT0004557 | 70.46 | 98.74 | 78.84 | 66.74 |
| hsa-miR-34a-5p | MIMAT0000255 | 37.93 | 39.04 | 68.79 | 75.93 |
| hsa-miR-34b-3p | MIMAT0004676 | 67.80 | 70.55 | 98.20 | 104.07 |
| hsa-miR-34b-5p | MIMAT0000685 | 84.31 | 73.10 | 132.29 | 128.37 |
| hsa-miR-34c-3p | MIMAT0004677 | 147.12 | 161.18 | 142.49 | 124.44 |
| hsa-miR-34c-5p | MIMAT0000686 | 33.46 | 39.89 | 80.23 | 91.66 |
| hsa-miR-3605-3p | MIMAT0017982 | 97.59 | 97.07 | 105.37 | 102.44 |
| hsa-miR-3605-5p | MIMAT0017981 | 102.06 | 100.90 | 118.63 | 102.92 |
| hsa-miR-3606-5p | MIMAT0017983 | 100.09 | 88.94 | 83.16 | 91.98 |
| hsa-miR-3607-3p | MIMAT0017985 | 67.69 | 101.66 | 95.93 | 127.87 |
| hsa-miR-3607-5p | MIMAT0017984 | 113.58 | 135.74 | 111.96 | 114.43 |
| hsa-miR-3609 | MIMAT0017986 | 106.30 | 109.48 | 109.49 | 105.28 |
| hsa-miR-3610 | MIMAT0017987 | 125.39 | 151.82 | 88.27 | 77.49 |
| hsa-miR-3611 | MIMAT0017988 | 96.95 | 90.18 | 86.00 | 83.11 |
| hsa-miR-3612 | MIMAT0017989 | 48.32 | 67.43 | 97.29 | 91.03 |
| hsa-miR-3613-3p | MIMAT0017991 | 101.42 | 92.48 | 106.73 | 98.81 |
| hsa-miR-3613-5p | MIMAT0017990 | 67.79 | 73.85 | 108.15 | 112.09 |
| hsa-miR-361-3p | MIMAT0004682 | 131.44 | 109.64 | 99.29 | 93.35 |
| hsa-miR-3614-3p | MIMAT0017993 | 127.90 | 118.96 | 113.37 | 114.52 |
| hsa-miR-3614-5p | MIMAT0017992 | 120.40 | 128.01 | 87.39 | 110.00 |
| hsa-miR-3615 | MIMAT0017994 | 166.78 | 162.80 | 134.76 | 151.73 |
| hsa-miR-361-5p | MIMAT0000703 | 91.08 | 102.49 | 97.84 | 97.84 |
| hsa-miR-3616-3p | MIMAT0017996 | 101.30 | 94.64 | 112.08 | 104.31 |
| hsa-miR-3616-5p | MIMAT0017995 | 132.55 | 145.68 | 88.55 | 80.55 |
| hsa-miR-3617-5p | MIMAT0017997 | 125.85 | 144.71 | 103.50 | 109.19 |
| hsa-miR-3618 | MIMAT0017998 | 119.16 | 137.73 | 110.67 | 127.90 |
| hsa-miR-3619-5p | MIMAT0017999 | 98.13 | 121.46 | 67.47 | 76.42 |
| hsa-miR-3620-3p | MIMAT0018001 | 113.57 | 126.62 | 100.18 | 112.83 |
| hsa-miR-3621 | MIMAT0018002 | 110.44 | 87.21 | 96.87 | 92.24 |
| hsa-miR-3622a-3p | MIMAT0018004 | 103.98 | 89.93 | 100.96 | 111.13 |
| hsa-miR-3622a-5p | MIMAT0018003 | 102.83 | 99.83 | 106.68 | 100.42 |
| hsa-miR-3622b-3p | MIMAT0018006 | 104.40 | 88.09 | 86.08 | 93.62 |
| hsa-miR-3622b-5p | MIMAT0018005 | 37.17 | 55.14 | 69.57 | 70.45 |
| hsa-miR-362-3p | MIMAT0004683 | 98.59 | 86.38 | 84.42 | 90.57 |
| hsa-miR-362-5p | MIMAT0000705 | 70.92 | 76.43 | 103.66 | 79.10 |
| hsa-miR-363-3p | MIMAT0000707 | 129.28 | 84.59 | 104.27 | 130.04 |
| hsa-miR-363-5p | MIMAT0003385 | 36.49 | 37.31 | 100.66 | 89.33 |
| hsa-miR-3646 | MIMAT0018065 | 116.58 | 67.71 | 90.67 | 87.29 |
| hsa-miR-3647-3p | MIMAT0018067 | 57.70 | 50.29 | 72.07 | 80.51 |
| hsa-miR-3647-5p | MIMAT0018066 | 137.02 | 129.37 | 120.90 | 117.62 |
| hsa-miR-3648 | MIMAT0018068 | 131.01 | 93.15 | 102.76 | 102.36 |
| hsa-miR-3649 | MIMAT0018069 | 135.51 | 134.47 | 104.27 | 105.38 |
| hsa-miR-365* | MIMAT0009199 | 83.51 | 75.96 | 108.04 | 95.95 |
| hsa-miR-3650 | MIMAT0018070 | 61.93 | 54.72 | 89.12 | 79.96 |
| hsa-miR-3651 | MIMAT0018071 | 71.53 | 68.86 | 124.78 | 117.51 |
| hsa-miR-3652 | MIMAT0018072 | 88.64 | 81.25 | 135.29 | 139.64 |
| hsa-miR-3653 | MIMAT0018073 | 147.98 | 155.08 | 95.20 | 87.14 |
| hsa-miR-3654 | MIMAT0018074 | 112.21 | 137.75 | 76.50 | 101.08 |
| hsa-miR-3655 | MIMAT0018075 | 117.39 | 96.81 | 121.69 | 93.26 |
| hsa-miR-3656 | MIMAT0018076 | 127.86 | 102.34 | 120.02 | 113.59 |
| hsa-miR-3657 | MIMAT0018077 | 51.79 | 47.88 | 98.84 | 92.24 |
| hsa-miR-3658 | MIMAT0018078 | 74.77 | 87.84 | 86.67 | 95.19 |
| hsa-miR-3659 | MIMAT0018080 | 110.05 | 114.67 | 107.07 | 92.05 |
| hsa-miR-365a-3p | MIMAT0000710 | 90.34 | 73.74 | 95.73 | 109.13 |
| hsa-miR-3660 | MIMAT0018081 | 86.57 | 92.08 | 69.76 | 81.69 |
| hsa-miR-3661 | MIMAT0018082 | 112.92 | 116.71 | 100.96 | 82.14 |
| hsa-miR-3662 | MIMAT0018083 | 87.05 | 93.24 | 92.80 | 96.16 |
| hsa-miR-3663-3p | MIMAT0018085 | 103.33 | 84.49 | 31.32 | 23.35 |
| hsa-miR-3663-5p | MIMAT0018084 | 94.66 | 112.02 | 92.89 | 89.09 |
| hsa-miR-3664-5p | MIMAT0018086 | 108.69 | 82.95 | 108.92 | 96.92 |
| hsa-miR-3665 | MIMAT0018087 | 3.89 | 19.52 | 98.40 | 85.53 |
| hsa-miR-3666 | MIMAT0018088 | 120.99 | 107.12 | 91.64 | 83.06 |
| hsa-miR-3667-3p | MIMAT0018090 | 67.88 | 62.37 | 92.46 | 71.03 |
| hsa-miR-3667-5p | MIMAT0018089 | 113.73 | 119.79 | 80.66 | 94.20 |
| hsa-miR-3668 | MIMAT0018091 | 99.82 | 107.27 | 103.68 | 103.16 |
| hsa-miR-3669 | MIMAT0018092 | 100.75 | 123.17 | 103.27 | 99.75 |
| hsa-miR-3670 | MIMAT0018093 | 107.40 | 105.03 | 89.03 | 93.02 |
| hsa-miR-3671 | MIMAT0018094 | 126.56 | 108.22 | 116.88 | 108.79 |
| hsa-miR-3672 | MIMAT0018095 | 109.40 | 132.11 | 114.06 | 124.81 |
| hsa-miR-3673 | MIMAT0018096 | 112.48 | 112.18 | 81.18 | 86.62 |
| hsa-miR-367-3p | MIMAT0000719 | 51.75 | 52.20 | 95.89 | 102.36 |
| hsa-miR-3674 | MIMAT0018097 | 57.75 | 59.08 | 88.17 | 80.59 |
| hsa-miR-3675-3p | MIMAT0018099 | 95.99 | 94.26 | 117.61 | 112.83 |
| hsa-miR-3675-5p | MIMAT0018098 | 124.45 | 136.85 | 105.37 | 109.44 |
| hsa-miR-367-5p | MIMAT0004686 | 96.08 | 93.38 | 85.57 | 63.36 |
| hsa-miR-3676-3p | MIMAT0018100 | 140.19 | 93.26 | 111.20 | 103.77 |
| hsa-miR-3677-3p | MIMAT0018101 | 102.38 | 102.94 | 98.92 | 100.02 |
| hsa-miR-3678-3p | MIMAT0018103 | 71.47 | 87.49 | 87.95 | 81.58 |
| hsa-miR-3678-5p | MIMAT0018102 | 106.21 | 104.72 | 95.52 | 84.56 |
| hsa-miR-3679-3p | MIMAT0018105 | 120.91 | 118.50 | 95.52 | 100.75 |
| hsa-miR-3679-5p | MIMAT0018104 | 69.92 | 77.92 | 94.50 | 71.10 |
| hsa-miR-3680-3p | MIMAT0018107 | 152.65 | 96.52 | 93.60 | 81.78 |
| hsa-miR-3680-5p | MIMAT0018106 | 94.07 | 104.21 | 113.19 | 100.02 |
| hsa-miR-3681-3p | MIMAT0018109 | 108.77 | 109.48 | 94.84 | 75.69 |
| hsa-miR-3681-5p | MIMAT0018108 | 68.54 | 68.32 | 114.40 | 106.98 |
| hsa-miR-3682-3p | MIMAT0018110 | 93.63 | 121.93 | 96.72 | 118.81 |
| hsa-miR-3683 | MIMAT0018111 | 88.96 | 94.26 | 93.14 | 87.46 |
| hsa-miR-3684 | MIMAT0018112 | 118.38 | 88.93 | 82.93 | 79.64 |
| hsa-miR-3685 | MIMAT0018113 | 102.06 | 101.66 | 90.42 | 114.52 |
| hsa-miR-3686 | MIMAT0018114 | 80.02 | 85.08 | 80.56 | 93.74 |
| hsa-miR-3687 | MIMAT0018115 | 112.73 | 119.13 | 118.45 | 110.54 |
| hsa-miR-3688-3p | MIMAT0018116 | 138.10 | 121.79 | 103.49 | 118.05 |
| hsa-miR-3689a-3p | MIMAT0018118 | 130.83 | 110.67 | 119.47 | 116.76 |
| hsa-miR-3689a-5p | MIMAT0018117 | 93.69 | 125.81 | 100.31 | 119.88 |
| hsa-miR-3689b-3p | MIMAT0018181 | 89.22 | 94.14 | 128.80 | 113.59 |
| hsa-miR-3689b-5p | MIMAT0018180 | 101.30 | 100.88 | 97.34 | 114.05 |
| hsa-miR-3690 | MIMAT0018119 | 91.75 | 83.49 | 104.92 | 116.48 |
| hsa-miR-3691-5p | MIMAT0018120 | 102.30 | 91.52 | 113.82 | 106.07 |
| hsa-miR-3692-3p | MIMAT0018122 | 93.57 | 131.08 | 112.22 | 114.07 |
| hsa-miR-3692-5p | MIMAT0018121 | 137.84 | 140.69 | 106.39 | 111.86 |
| hsa-miR-369-3p | MIMAT0000721 | 116.11 | 113.15 | 138.23 | 155.59 |
| hsa-miR-369-5p | MIMAT0001621 | 105.87 | 108.74 | 116.98 | 115.99 |
| hsa-miR-370 | MIMAT0000722 | 92.72 | 101.48 | 101.47 | 97.85 |
| hsa-miR-3713 | MIMAT0018164 | 81.53 | 70.60 | 106.15 | 102.48 |
| hsa-miR-3714 | MIMAT0018165 | 68.39 | 74.18 | 101.17 | 87.86 |
| hsa-miR-371a-3p | MIMAT0000723 | 135.14 | 135.97 | 129.29 | 128.84 |
| hsa-miR-371a-5p | MIMAT0004687 | 139.61 | 137.14 | 101.10 | 110.15 |
| hsa-miR-372 | MIMAT0000724 | 86.91 | 115.93 | 121.86 | 128.30 |
| hsa-miR-373-3p | MIMAT0000726 | 139.07 | 135.84 | 138.34 | 158.15 |
| hsa-miR-373-5p | MIMAT0000725 | 151.73 | 161.56 | 111.33 | 92.81 |
| hsa-miR-374a-3p | MIMAT0004688 | 80.02 | 97.14 | 82.37 | 97.39 |
| hsa-miR-374a-5p | MIMAT0000727 | 87.82 | 96.20 | 114.49 | 84.95 |
| hsa-miR-374b-3p | MIMAT0004956 | 49.17 | 42.60 | 114.46 | 90.70 |
| hsa-miR-374b-5p | MIMAT0004955 | 64.48 | 70.12 | 116.79 | 117.31 |
| hsa-miR-374c-5p | MIMAT0018443 | 60.53 | 57.53 | 96.54 | 93.74 |
| hsa-miR-375 | MIMAT0000728 | 115.48 | 101.41 | 97.56 | 93.74 |
| hsa-miR-376a-3p | MIMAT0000729 | 77.76 | 102.14 | 94.06 | 84.77 |
| hsa-miR-376a-5p | MIMAT0003386 | 104.10 | 94.86 | 84.07 | 96.59 |
| hsa-miR-376b-3p | MIMAT0002172 | 111.64 | 104.67 | 102.79 | 87.26 |
| hsa-miR-376c-3p | MIMAT0000720 | 86.18 | 69.76 | 82.27 | 67.03 |
| hsa-miR-377-3p | MIMAT0000730 | 98.23 | 106.76 | 93.14 | 109.20 |
| hsa-miR-377-5p | MIMAT0004689 | 57.65 | 56.03 | 76.09 | 69.78 |
| hsa-miR-378a-3p | MIMAT0000732 | 105.43 | 116.51 | 111.95 | 112.54 |
| hsa-miR-378a-5p | MIMAT0000731 | 76.95 | 105.18 | 98.12 | 106.33 |
| hsa-miR-378b | MIMAT0014999 | 135.51 | 142.06 | 125.60 | 133.26 |
| hsa-miR-378c | MIMAT0016847 | 106.30 | 91.63 | 101.17 | 110.82 |
| hsa-miR-379-3p | MIMAT0004690 | 122.50 | 136.81 | 102.48 | 102.16 |
| hsa-miR-379-5p | MIMAT0000733 | 98.45 | 88.91 | 69.61 | 60.31 |
| hsa-miR-380-3p | MIMAT0000735 | 82.26 | 82.76 | 97.85 | 94.58 |
| hsa-miR-380-5p | MIMAT0000734 | 65.49 | 58.77 | 134.01 | 130.53 |
| hsa-miR-381-3p | MIMAT0000736 | 104.62 | 91.26 | 126.82 | 110.46 |
| hsa-miR-382-5p | MIMAT0000737 | 97.14 | 87.75 | 102.56 | 98.51 |
| hsa-miR-383 | MIMAT0000738 | 49.96 | 61.66 | 125.15 | 132.29 |
| hsa-miR-384 | MIMAT0001075 | 92.00 | 117.16 | 74.67 | 84.91 |
| hsa-miR-3907 | MIMAT0018179 | 49.80 | 49.98 | 81.21 | 88.26 |
| hsa-miR-3908 | MIMAT0018182 | 71.96 | 89.05 | 96.39 | 73.13 |
| hsa-miR-3909 | MIMAT0018183 | 126.95 | 142.06 | 80.80 | 85.00 |
| hsa-miR-3910 | MIMAT0018184 | 81.97 | 61.29 | 81.32 | 99.55 |
| hsa-miR-3911 | MIMAT0018185 | 73.21 | 57.48 | 97.33 | 106.98 |
| hsa-miR-3912 | MIMAT0018186 | 96.86 | 118.84 | 86.55 | 116.34 |
| hsa-miR-3913-5p | MIMAT0018187 | 125.31 | 115.46 | 108.72 | 103.35 |
| hsa-miR-3914 | MIMAT0018188 | 119.95 | 112.63 | 105.04 | 120.32 |
| hsa-miR-3915 | MIMAT0018189 | 97.89 | 101.57 | 93.08 | 90.55 |
| hsa-miR-3916 | MIMAT0018190 | 74.50 | 90.71 | 106.23 | 122.56 |
| hsa-miR-3917 | MIMAT0018191 | 107.79 | 93.61 | 101.73 | 123.09 |
| hsa-miR-3918 | MIMAT0018192 | 72.78 | 87.36 | 91.69 | 85.87 |
| hsa-miR-3919 | MIMAT0018193 | 78.10 | 71.66 | 86.92 | 107.97 |
| hsa-miR-3920 | MIMAT0018195 | 123.81 | 122.45 | 103.97 | 83.26 |
| hsa-miR-3921 | MIMAT0018196 | 82.51 | 103.53 | 98.95 | 105.67 |
| hsa-miR-3922-3p | MIMAT0018197 | 75.55 | 116.04 | 105.33 | 80.17 |
| hsa-miR-3923 | MIMAT0018198 | 86.15 | 79.32 | 98.84 | 102.15 |
| hsa-miR-3924 | MIMAT0018199 | 107.57 | 88.52 | 92.16 | 84.36 |
| hsa-miR-3925-5p | MIMAT0018200 | 87.28 | 90.12 | 117.40 | 99.17 |
| hsa-miR-3926 | MIMAT0018201 | 116.71 | 119.00 | 97.29 | 112.80 |
| hsa-miR-3927-3p | MIMAT0018202 | 96.95 | 90.95 | 82.26 | 92.53 |
| hsa-miR-3928 | MIMAT0018205 | 83.26 | 101.94 | 101.73 | 94.20 |
| hsa-miR-3929 | MIMAT0018206 | 96.57 | 93.26 | 90.93 | 103.23 |
| hsa-miR-3934-5p | MIMAT0018349 | 95.89 | 99.17 | 109.49 | 105.67 |
| hsa-miR-3935 | MIMAT0018350 | 67.87 | 59.83 | 77.45 | 69.89 |
| hsa-miR-3936 | MIMAT0018351 | 83.79 | 96.86 | 84.61 | 106.26 |
| hsa-miR-3937 | MIMAT0018352 | 89.12 | 117.65 | 103.25 | 89.91 |
| hsa-miR-3938 | MIMAT0018353 | 94.07 | 116.71 | 90.08 | 90.60 |
| hsa-miR-3939 | MIMAT0018355 | 91.43 | 83.30 | 118.36 | 113.19 |
| hsa-miR-3940-3p | MIMAT0018356 | 131.99 | 111.31 | 103.03 | 123.37 |
| hsa-miR-3941 | MIMAT0018357 | 79.70 | 87.38 | 81.24 | 81.18 |
| hsa-miR-3942-5p | MIMAT0018358 | 86.68 | 94.90 | 93.44 | 87.25 |
| hsa-miR-3943 | MIMAT0018359 | 115.78 | 91.67 | 114.53 | 113.65 |
| hsa-miR-3944-3p | MIMAT0018360 | 128.50 | 147.49 | 83.47 | 72.66 |
| hsa-miR-3945 | MIMAT0018361 | 105.11 | 91.67 | 99.33 | 117.54 |
| hsa-miR-409-3p | MIMAT0001639 | 89.12 | 94.64 | 68.91 | 79.01 |
| hsa-miR-409-5p | MIMAT0001638 | 101.74 | 98.34 | 91.44 | 94.22 |
| hsa-miR-410 | MIMAT0002171 | 86.25 | 78.13 | 120.83 | 119.59 |
| hsa-miR-411-3p | MIMAT0004813 | 113.07 | 110.81 | 118.49 | 112.90 |
| hsa-miR-411-5p | MIMAT0003329 | 92.18 | 96.39 | 99.51 | 96.18 |
| hsa-miR-412 | MIMAT0002170 | 117.10 | 74.16 | 69.14 | 75.67 |
| hsa-miR-421 | MIMAT0003339 | 104.26 | 84.09 | 86.65 | 67.60 |
| hsa-miR-422a | MIMAT0001339 | 132.40 | 119.29 | 143.20 | 146.67 |
| hsa-miR-423-3p | MIMAT0001340 | 104.04 | 95.64 | 95.89 | 82.26 |
| hsa-miR-423-5p | MIMAT0004748 | 89.15 | 82.78 | 126.58 | 115.28 |
| hsa-miR-424-3p | MIMAT0004749 | 74.39 | 67.76 | 121.21 | 120.82 |
| hsa-miR-424-5p | MIMAT0001341 | 81.40 | 85.91 | 86.20 | 83.08 |
| hsa-miR-4251 | MIMAT0016883 | 121.17 | 108.29 | 82.88 | 100.53 |
| hsa-miR-4252 | MIMAT0016886 | 89.70 | 83.49 | 101.60 | 112.83 |
| hsa-miR-4253 | MIMAT0016882 | 63.86 | 50.97 | 96.80 | 80.17 |
| hsa-miR-425-3p | MIMAT0001343 | 85.25 | 77.76 | 97.34 | 77.81 |
| hsa-miR-4254 | MIMAT0016884 | 88.79 | 68.32 | 81.87 | 81.24 |
| hsa-miR-4255 | MIMAT0016885 | 81.51 | 84.88 | 110.13 | 101.49 |
| hsa-miR-425-5p | MIMAT0003393 | 50.61 | 74.16 | 92.31 | 101.25 |
| hsa-miR-4256 | MIMAT0016877 | 67.19 | 73.75 | 90.24 | 80.42 |
| hsa-miR-4257 | MIMAT0016878 | 114.60 | 102.85 | 103.96 | 87.77 |
| hsa-miR-4258 | MIMAT0016879 | 92.18 | 86.47 | 87.87 | 102.51 |
| hsa-miR-4259 | MIMAT0016880 | 88.45 | 67.99 | 122.59 | 119.47 |
| hsa-miR-4260 | MIMAT0016881 | 115.22 | 83.30 | 100.62 | 100.53 |
| hsa-miR-4261 | MIMAT0016890 | 132.67 | 140.53 | 147.55 | 152.13 |
| hsa-miR-4262 | MIMAT0016894 | 102.38 | 119.01 | 90.08 | 92.77 |
| hsa-miR-4263 | MIMAT0016898 | 100.80 | 119.86 | 94.67 | 89.41 |
| hsa-miR-4264 | MIMAT0016899 | 78.10 | 80.70 | 129.08 | 103.92 |
| hsa-miR-4265 | MIMAT0016891 | 51.79 | 67.17 | 116.49 | 117.54 |
| hsa-miR-4266 | MIMAT0016892 | 86.83 | 71.63 | 108.65 | 96.14 |
| hsa-miR-4267 | MIMAT0016893 | 93.69 | 89.44 | 69.41 | 76.29 |
| hsa-miR-4268 | MIMAT0016896 | 109.22 | 95.01 | 82.76 | 85.15 |
| hsa-miR-4269 | MIMAT0016897 | 99.78 | 102.06 | 84.61 | 105.09 |
| hsa-miR-4270 | MIMAT0016900 | 89.70 | 92.53 | 115.34 | 103.92 |
| hsa-miR-4271 | MIMAT0016901 | 84.61 | 63.63 | 70.70 | 65.87 |
| hsa-miR-4272 | MIMAT0016902 | 108.92 | 68.53 | 94.99 | 75.29 |
| hsa-miR-4273 | MIMAT0016903 | 100.35 | 95.60 | 113.92 | 106.07 |
| hsa-miR-4274 | MIMAT0016906 | 103.59 | 104.29 | 80.69 | 70.84 |
| hsa-miR-4275 | MIMAT0016905 | 94.31 | 100.27 | 97.76 | 108.31 |
| hsa-miR-4276 | MIMAT0016904 | 63.86 | 66.15 | 79.73 | 75.88 |
| hsa-miR-4277 | MIMAT0016908 | 87.95 | 84.97 | 88.38 | 98.17 |
| hsa-miR-4278 | MIMAT0016910 | 80.76 | 68.86 | 89.34 | 94.96 |
| hsa-miR-4279 | MIMAT0016909 | 145.42 | 133.13 | 129.58 | 146.11 |
| hsa-miR-4280 | MIMAT0016911 | 111.97 | 119.13 | 102.27 | 114.43 |
| hsa-miR-4281 | MIMAT0016907 | 109.68 | 108.00 | 115.02 | 107.81 |
| hsa-miR-4282 | MIMAT0016912 | 100.64 | 100.32 | 78.00 | 96.12 |
| hsa-miR-4283 | MIMAT0016914 | 109.27 | 113.05 | 90.08 | 106.86 |
| hsa-miR-4284 | MIMAT0016915 | 116.91 | 115.06 | 111.99 | 132.55 |
| hsa-miR-4285 | MIMAT0016913 | 104.07 | 100.75 | 117.25 | 113.19 |
| hsa-miR-4286 | MIMAT0016916 | 145.18 | 148.60 | 124.41 | 100.02 |
| hsa-miR-4287 | MIMAT0016917 | 93.46 | 104.11 | 99.47 | 101.62 |
| hsa-miR-4288 | MIMAT0016918 | 72.19 | 80.86 | 73.85 | 82.87 |
| hsa-miR-4289 | MIMAT0016920 | 116.75 | 111.36 | 95.52 | 109.93 |
| hsa-miR-429 | MIMAT0001536 | 55.60 | 50.10 | 77.25 | 54.10 |
| hsa-miR-4290 | MIMAT0016921 | 66.51 | 91.83 | 103.97 | 117.29 |
| hsa-miR-4291 | MIMAT0016922 | 78.45 | 92.41 | 84.12 | 106.26 |
| hsa-miR-4292 | MIMAT0016919 | 130.76 | 155.15 | 110.90 | 126.01 |
| hsa-miR-4293 | MIMAT0016848 | 67.87 | 68.18 | 69.39 | 75.56 |
| hsa-miR-4294 | MIMAT0016849 | 125.63 | 113.84 | 118.91 | 87.86 |
| hsa-miR-4295 | MIMAT0016844 | 137.45 | 114.79 | 123.87 | 97.44 |
| hsa-miR-4296 | MIMAT0016845 | 75.55 | 66.15 | 105.33 | 109.67 |
| hsa-miR-4297 | MIMAT0016846 | 121.08 | 86.27 | 108.24 | 137.95 |
| hsa-miR-4298 | MIMAT0016852 | 126.37 | 110.67 | 131.11 | 123.09 |
| hsa-miR-4299 | MIMAT0016851 | 121.17 | 129.71 | 93.41 | 112.01 |
| hsa-miR-4300 | MIMAT0016853 | 110.02 | 102.74 | 102.83 | 89.05 |
| hsa-miR-4301 | MIMAT0016850 | 104.07 | 115.83 | 112.81 | 98.95 |
| hsa-miR-4302 | MIMAT0016855 | 105.21 | 123.41 | 66.19 | 84.93 |
| hsa-miR-4303 | MIMAT0016856 | 80.28 | 89.25 | 31.88 | 36.02 |
| hsa-miR-4304 | MIMAT0016854 | 104.02 | 94.62 | 105.87 | 115.26 |
| hsa-miR-4305 | MIMAT0016857 | 69.24 | 86.27 | 99.71 | 82.85 |
| hsa-miR-4306 | MIMAT0016858 | 81.00 | 62.90 | 92.53 | 100.55 |
| hsa-miR-4307 | MIMAT0016860 | 99.34 | 79.64 | 99.25 | 90.75 |
| hsa-miR-4308 | MIMAT0016861 | 88.96 | 94.77 | 111.49 | 90.36 |
| hsa-miR-4309 | MIMAT0016859 | 86.23 | 83.70 | 101.73 | 94.59 |
| hsa-miR-4310 | MIMAT0016862 | 107.48 | 82.42 | 111.73 | 99.48 |
| hsa-miR-4311 | MIMAT0016863 | 105.13 | 105.57 | 111.09 | 111.17 |
| hsa-miR-4312 | MIMAT0016864 | 66.28 | 62.63 | 104.02 | 92.29 |
| hsa-miR-4313 | MIMAT0016865 | 122.86 | 124.31 | 115.82 | 109.18 |
| hsa-miR-431-3p | MIMAT0004757 | 79.00 | 65.89 | 112.33 | 117.31 |
| hsa-miR-4314 | MIMAT0016868 | 102.07 | 105.03 | 74.31 | 75.51 |
| hsa-miR-4315 | MIMAT0016866 | 93.75 | 95.28 | 117.61 | 85.53 |
| hsa-miR-431-5p | MIMAT0001625 | 74.01 | 53.57 | 98.76 | 84.88 |
| hsa-miR-4316 | MIMAT0016867 | 29.73 | 76.16 | 93.96 | 81.14 |
| hsa-miR-4317 | MIMAT0016872 | 95.23 | 112.38 | 100.09 | 95.03 |
| hsa-miR-4318 | MIMAT0016869 | 114.25 | 120.62 | 84.61 | 93.80 |
| hsa-miR-4319 | MIMAT0016870 | 89.02 | 84.88 | 96.87 | 97.03 |
| hsa-miR-4320 | MIMAT0016871 | 60.75 | 79.16 | 82.40 | 84.46 |
| hsa-miR-4321 | MIMAT0016874 | 116.14 | 112.44 | 103.96 | 132.55 |
| hsa-miR-4322 | MIMAT0016873 | 103.84 | 95.01 | 109.07 | 99.86 |
| hsa-miR-4323 | MIMAT0016875 | 93.46 | 80.25 | 120.27 | 118.78 |
| hsa-miR-432-3p | MIMAT0002815 | 119.74 | 129.95 | 127.80 | 98.41 |
| hsa-miR-4324 | MIMAT0016876 | 119.99 | 121.16 | 102.49 | 100.51 |
| hsa-miR-4325 | MIMAT0016887 | 81.50 | 93.15 | 96.38 | 112.87 |
| hsa-miR-432-5p | MIMAT0002814 | 137.46 | 138.43 | 101.47 | 88.82 |
| hsa-miR-4326 | MIMAT0016888 | 42.06 | 47.72 | 92.00 | 84.46 |
| hsa-miR-4327 | MIMAT0016889 | 130.76 | 110.70 | 94.46 | 79.92 |
| hsa-miR-4328 | MIMAT0016926 | 95.66 | 98.78 | 72.21 | 87.93 |
| hsa-miR-4329 | MIMAT0016923 | 127.11 | 119.40 | 78.44 | 87.47 |
| hsa-miR-433 | MIMAT0001627 | 113.56 | 98.09 | 89.06 | 98.09 |
| hsa-miR-4330 | MIMAT0016924 | 81.77 | 60.69 | 91.75 | 103.70 |
| hsa-miR-448 | MIMAT0001532 | 99.73 | 111.45 | 114.07 | 110.13 |
| hsa-miR-449a | MIMAT0001541 | 21.82 | 27.24 | 45.59 | 62.93 |
| hsa-miR-449b-3p | MIMAT0009203 | 103.77 | 94.79 | 131.84 | 107.89 |
| hsa-miR-449b-5p | MIMAT0003327 | 36.17 | 26.71 | 65.57 | 60.88 |
| hsa-miR-449c-3p | MIMAT0013771 | 145.70 | 142.40 | 101.73 | 105.67 |
| hsa-miR-449c-5p | MIMAT0010251 | 91.29 | 66.05 | 94.37 | 84.42 |
| hsa-miR-450a-5p | MIMAT0001545 | 169.53 | 177.24 | 156.37 | 178.96 |
| hsa-miR-450b-3p | MIMAT0004910 | 95.41 | 92.40 | 98.49 | 107.28 |
| hsa-miR-450b-5p | MIMAT0004909 | 36.30 | 34.18 | 86.13 | 94.62 |
| hsa-miR-451a | MIMAT0001631 | 80.71 | 89.15 | 96.72 | 133.15 |
| hsa-miR-452-3p | MIMAT0001636 | 86.91 | 92.39 | 101.40 | 84.50 |
| hsa-miR-452-5p | MIMAT0001635 | 136.07 | 110.20 | 85.24 | 87.48 |
| hsa-miR-453 | MIMAT0001630 | 102.07 | 99.83 | 116.00 | 128.83 |
| hsa-miR-454-3p | MIMAT0003885 | 133.80 | 134.07 | 102.83 | 106.86 |
| hsa-miR-454-5p | MIMAT0003884 | 129.91 | 129.36 | 143.18 | 158.56 |
| hsa-miR-455-3p | MIMAT0004784 | 129.22 | 126.39 | 86.78 | 115.88 |
| hsa-miR-455-5p | MIMAT0003150 | 94.45 | 75.34 | 126.30 | 112.87 |
| hsa-miR-466 | MIMAT0015002 | 102.26 | 111.18 | 87.04 | 88.59 |
| hsa-miR-483-3p | MIMAT0002173 | 128.29 | 126.11 | 96.63 | 95.38 |
| hsa-miR-483-5p | MIMAT0004761 | 87.08 | 90.20 | 119.87 | 107.38 |
| hsa-miR-484 | MIMAT0002174 | 150.67 | 155.24 | 100.29 | 108.68 |
| hsa-miR-485-3p | MIMAT0002176 | 117.43 | 134.25 | 94.49 | 131.44 |
| hsa-miR-485-5p | MIMAT0002175 | 68.03 | 76.05 | 111.06 | 125.66 |
| hsa-miR-486-3p | MIMAT0004762 | 96.58 | 87.82 | 111.05 | 115.17 |
| hsa-miR-486-5p | MIMAT0002177 | 157.32 | 116.04 | 109.60 | 104.30 |
| hsa-miR-487a | MIMAT0002178 | 101.88 | 117.04 | 95.51 | 92.10 |
| hsa-miR-487b | MIMAT0003180 | 137.84 | 139.42 | 114.89 | 120.07 |
| hsa-miR-488-3p | MIMAT0004763 | 109.93 | 95.32 | 102.79 | 110.63 |
| hsa-miR-488-5p | MIMAT0002804 | 86.25 | 100.83 | 108.17 | 88.82 |
| hsa-miR-489 | MIMAT0002805 | 115.96 | 87.66 | 107.82 | 93.01 |
| hsa-miR-490-3p | MIMAT0002806 | 120.76 | 107.21 | 106.88 | 117.51 |
| hsa-miR-490-5p | MIMAT0004764 | 57.96 | 61.77 | 101.37 | 97.88 |
| hsa-miR-491-3p | MIMAT0004765 | 159.71 | 132.00 | 88.08 | 101.90 |
| hsa-miR-491-5p | MIMAT0002807 | 117.07 | 88.40 | 102.32 | 99.78 |
| hsa-miR-492 | MIMAT0002812 | 92.16 | 105.77 | 95.89 | 109.37 |
| hsa-miR-493-3p | MIMAT0003161 | 153.63 | 201.98 | 117.11 | 123.28 |
| hsa-miR-493-5p | MIMAT0002813 | 101.74 | 92.99 | 109.45 | 100.99 |
| hsa-miR-494 | MIMAT0002816 | 82.37 | 70.77 | 137.80 | 136.08 |
| hsa-miR-495-3p | MIMAT0002817 | 92.70 | 86.00 | 102.44 | 81.54 |
| hsa-miR-496 | MIMAT0002818 | 105.48 | 110.99 | 120.69 | 107.03 |
| hsa-miR-497-3p | MIMAT0004768 | 91.66 | 85.42 | 112.75 | 72.08 |
| hsa-miR-497-5p | MIMAT0002820 | 63.98 | 64.95 | 70.39 | 75.90 |
| hsa-miR-498 | MIMAT0002824 | 62.30 | 70.60 | 96.28 | 109.01 |
| hsa-miR-499a-3p | MIMAT0004772 | 89.25 | 69.32 | 117.05 | 97.13 |
| hsa-miR-499a-5p | MIMAT0002870 | 119.13 | 111.85 | 85.46 | 88.41 |
| hsa-miR-500a-3p | MIMAT0002871 | 95.38 | 101.98 | 98.84 | 119.47 |
| hsa-miR-500a-5p | MIMAT0004773 | 177.47 | 179.25 | 120.91 | 117.15 |
| hsa-miR-500b | MIMAT0016925 | 90.69 | 104.32 | 97.29 | 99.74 |
| hsa-miR-501-3p | MIMAT0004774 | 160.99 | 168.08 | 89.47 | 97.61 |
| hsa-miR-501-5p | MIMAT0002872 | 113.18 | 101.18 | 86.50 | 92.84 |
| hsa-miR-502-3p | MIMAT0004775 | 85.83 | 87.74 | 120.13 | 121.16 |
| hsa-miR-502-5p | MIMAT0002873 | 82.88 | 82.25 | 77.36 | 80.68 |
| hsa-miR-503-5p | MIMAT0002874 | 57.78 | 71.44 | 67.78 | 77.43 |
| hsa-miR-504 | MIMAT0002875 | 83.53 | 86.10 | 105.04 | 95.19 |
| hsa-miR-505-3p | MIMAT0002876 | 108.66 | 99.59 | 115.06 | 121.02 |
| hsa-miR-505-5p | MIMAT0004776 | 89.48 | 77.48 | 111.52 | 103.18 |
| hsa-miR-506-3p | MIMAT0002878 | 133.97 | 129.18 | 143.52 | 148.45 |
| hsa-miR-507 | MIMAT0002879 | 58.04 | 68.11 | 78.87 | 76.11 |
| hsa-miR-508-3p | MIMAT0002880 | 102.10 | 144.70 | 109.86 | 74.53 |
| hsa-miR-508-5p | MIMAT0004778 | 32.03 | 46.16 | 75.53 | 59.94 |
| hsa-miR-509-3-5p | MIMAT0004975 | 71.69 | 63.10 | 134.57 | 107.57 |
| hsa-miR-509-3p | MIMAT0002881 | 111.32 | 105.74 | 114.21 | 114.76 |
| hsa-miR-509-5p | MIMAT0004779 | 35.04 | 32.29 | 129.14 | 130.42 |
| hsa-miR-510 | MIMAT0002882 | 36.15 | 43.58 | 87.51 | 115.88 |
| hsa-miR-511 | MIMAT0002808 | 139.11 | 125.92 | 116.77 | 97.31 |
| hsa-miR-512-3p | MIMAT0002823 | 156.83 | 167.92 | 95.63 | 99.30 |
| hsa-miR-512-5p | MIMAT0002822 | 116.50 | 85.27 | 76.32 | 59.02 |
| hsa-miR-513a-3p | MIMAT0004777 | 121.07 | 100.43 | 107.66 | 69.81 |
| hsa-miR-513a-5p | MIMAT0002877 | 73.99 | 79.16 | 85.07 | 83.39 |
| hsa-miR-513b | MIMAT0005788 | 82.38 | 104.36 | 75.16 | 98.36 |
| hsa-miR-513c-5p | MIMAT0005789 | 118.20 | 108.17 | 129.61 | 129.02 |
| hsa-miR-514a-3p | MIMAT0002883 | 71.42 | 66.15 | 87.06 | 76.14 |
| hsa-miR-514b-3p | MIMAT0015088 | 89.12 | 99.83 | 82.65 | 87.18 |
| hsa-miR-514b-5p | MIMAT0015087 | 136.03 | 112.65 | 109.49 | 110.03 |
| hsa-miR-515-3p | MIMAT0002827 | 174.45 | 173.51 | 119.73 | 118.78 |
| hsa-miR-515-5p | MIMAT0002826 | 120.57 | 88.57 | 70.82 | 104.45 |
| hsa-miR-516a-3p | MIMAT0002860 | 98.63 | 99.52 | 94.91 | 113.53 |
| hsa-miR-516a-5p | MIMAT0004770 | 121.17 | 102.34 | 112.26 | 97.36 |
| hsa-miR-516b-5p | MIMAT0002859 | 118.46 | 94.27 | 75.95 | 67.73 |
| hsa-miR-517-5p | MIMAT0002851 | 122.36 | 98.88 | 103.33 | 107.69 |
| hsa-miR-517a-3p | MIMAT0002852 | 76.44 | 99.01 | 61.60 | 42.38 |
| hsa-miR-517b | MIMAT0002857 | 150.89 | 144.26 | 107.34 | 122.39 |
| hsa-miR-517c-3p | MIMAT0002866 | 87.25 | 85.64 | 128.09 | 105.51 |
| hsa-miR-518a-3p | MIMAT0002863 | 99.79 | 85.50 | 104.04 | 90.63 |
| hsa-miR-518a-5p | MIMAT0005457 | 22.37 | 17.67 | 59.23 | 41.25 |
| hsa-miR-518b | MIMAT0002844 | 109.27 | 89.65 | 85.65 | 92.22 |
| hsa-miR-518c-3p | MIMAT0002848 | 101.10 | 133.28 | 102.83 | 108.05 |
| hsa-miR-518c-5p | MIMAT0002847 | 107.03 | 91.11 | 95.78 | 97.19 |
| hsa-miR-518d-3p | MIMAT0002864 | 84.89 | 113.87 | 98.93 | 67.30 |
| hsa-miR-518d-5p | MIMAT0005456 | 144.45 | 145.19 | 120.18 | 127.57 |
| hsa-miR-518e-3p | MIMAT0002861 | 116.49 | 98.12 | 118.83 | 117.92 |
| hsa-miR-518e-5p | MIMAT0005450 | 92.67 | 76.46 | 82.22 | 112.60 |
| hsa-miR-518f-3p | MIMAT0002842 | 100.65 | 89.88 | 80.88 | 113.61 |
| hsa-miR-518f-5p | MIMAT0002841 | 106.81 | 113.45 | 85.45 | 87.29 |
| hsa-miR-519a-3p | MIMAT0002869 | 125.97 | 131.43 | 137.97 | 161.83 |
| hsa-miR-519b-3p | MIMAT0002837 | 134.15 | 120.33 | 115.46 | 124.00 |
| hsa-miR-519c-3p | MIMAT0002832 | 113.20 | 121.58 | 114.13 | 116.31 |
| hsa-miR-519d | MIMAT0002853 | 80.76 | 86.29 | 124.42 | 114.57 |
| hsa-miR-519e-3p | MIMAT0002829 | 99.19 | 122.33 | 82.26 | 76.10 |
| hsa-miR-519e-5p | MIMAT0002828 | 108.53 | 106.16 | 68.25 | 73.52 |
| hsa-miR-520a-3p | MIMAT0002834 | 137.73 | 136.43 | 138.20 | 151.71 |
| hsa-miR-520a-5p | MIMAT0002833 | 37.98 | 45.06 | 83.22 | 78.56 |
| hsa-miR-520b | MIMAT0002843 | 97.08 | 118.93 | 112.27 | 134.03 |
| hsa-miR-520c-3p | MIMAT0002846 | 106.85 | 119.01 | 122.03 | 103.16 |
| hsa-miR-520d-3p | MIMAT0002856 | 166.82 | 203.57 | 132.14 | 136.45 |
| hsa-miR-520d-5p | MIMAT0002855 | 162.63 | 147.31 | 104.33 | 87.81 |
| hsa-miR-520e | MIMAT0002825 | 100.08 | 101.57 | 94.08 | 123.82 |
| hsa-miR-520f | MIMAT0002830 | 165.34 | 152.49 | 164.35 | 140.85 |
| hsa-miR-520g | MIMAT0002858 | 125.60 | 107.31 | 114.13 | 118.77 |
| hsa-miR-520h | MIMAT0002867 | 78.13 | 84.50 | 89.97 | 88.43 |
| hsa-miR-521 | MIMAT0002854 | 101.53 | 90.65 | 78.01 | 102.80 |
| hsa-miR-522-3p | MIMAT0002868 | 46.90 | 31.45 | 117.11 | 110.56 |
| hsa-miR-523-3p | MIMAT0002840 | 101.58 | 100.63 | 115.05 | 107.89 |
| hsa-miR-524-3p | MIMAT0002850 | 62.30 | 63.63 | 81.67 | 92.34 |
| hsa-miR-524-5p | MIMAT0002849 | 127.02 | 106.37 | 119.06 | 109.29 |
| hsa-miR-525-3p | MIMAT0002839 | 93.83 | 117.46 | 116.10 | 141.40 |
| hsa-miR-525-5p | MIMAT0002838 | 111.66 | 129.42 | 107.17 | 95.06 |
| hsa-miR-526b-3p | MIMAT0002836 | 176.36 | 184.28 | 86.13 | 110.03 |
| hsa-miR-526b-5p | MIMAT0002835 | 63.86 | 92.18 | 109.07 | 100.01 |
| hsa-miR-532-3p | MIMAT0004780 | 112.93 | 92.18 | 77.60 | 75.35 |
| hsa-miR-532-5p | MIMAT0002888 | 137.87 | 163.67 | 120.42 | 117.54 |
| hsa-miR-539-5p | MIMAT0003163 | 140.97 | 167.01 | 109.60 | 117.17 |
| hsa-miR-541-3p | MIMAT0004920 | 64.74 | 57.94 | 120.81 | 124.53 |
| hsa-miR-541-5p | MIMAT0004919 | 116.50 | 97.56 | 111.06 | 108.53 |
| hsa-miR-542-3p | MIMAT0003389 | 58.49 | 67.39 | 81.94 | 95.48 |
| hsa-miR-542-5p | MIMAT0003340 | 94.13 | 105.24 | 103.09 | 106.62 |
| hsa-miR-543 | MIMAT0004954 | 88.46 | 128.52 | 103.39 | 101.72 |
| hsa-miR-544a | MIMAT0003164 | 51.10 | 50.43 | 107.64 | 87.16 |
| hsa-miR-544b | MIMAT0015004 | 92.43 | 62.61 | 92.60 | 80.02 |
| hsa-miR-545-3p | MIMAT0003165 | 141.67 | 113.87 | 97.44 | 91.55 |
| hsa-miR-545-5p | MIMAT0004785 | 87.58 | 97.14 | 68.67 | 75.43 |
| hsa-miR-548a-3p | MIMAT0003251 | 118.09 | 97.04 | 134.19 | 110.36 |
| hsa-miR-548a-5p | MIMAT0004803 | 101.58 | 103.71 | 108.79 | 104.72 |
| hsa-miR-548aa | MIMAT0018447 | 63.78 | 75.14 | 102.08 | 104.73 |
| hsa-miR-548b-3p | MIMAT0003254 | 105.89 | 84.06 | 92.80 | 107.03 |
| hsa-miR-548b-5p | MIMAT0004798 | 45.67 | 53.01 | 115.05 | 102.82 |
| hsa-miR-548c-3p | MIMAT0003285 | 97.03 | 74.89 | 99.24 | 83.90 |
| hsa-miR-548c-5p | MIMAT0004806 | 68.64 | 69.55 | 113.10 | 123.73 |
| hsa-miR-548d-3p | MIMAT0003323 | 116.12 | 111.10 | 96.20 | 115.00 |
| hsa-miR-548d-5p | MIMAT0004812 | 113.16 | 113.11 | 77.21 | 86.91 |
| hsa-miR-548e | MIMAT0005874 | 145.78 | 153.49 | 110.18 | 119.82 |
| hsa-miR-548f | MIMAT0005895 | 124.25 | 108.41 | 106.76 | 94.04 |
| hsa-miR-548g-3p | MIMAT0005912 | 105.04 | 105.26 | 74.24 | 91.18 |
| hsa-miR-548h-5p | MIMAT0005928 | 94.87 | 111.85 | 97.00 | 96.21 |
| hsa-miR-548i | MIMAT0005935 | 124.92 | 105.03 | 110.61 | 107.81 |
| hsa-miR-548j | MIMAT0005875 | 86.87 | 84.89 | 83.43 | 76.72 |
| hsa-miR-548k | MIMAT0005882 | 87.44 | 83.31 | 121.86 | 127.96 |
| hsa-miR-548l | MIMAT0005889 | 89.33 | 97.14 | 100.34 | 116.48 |
| hsa-miR-548m | MIMAT0005917 | 138.79 | 137.26 | 117.45 | 105.50 |
| hsa-miR-548n | MIMAT0005916 | 69.21 | 64.79 | 114.78 | 124.00 |
| hsa-miR-548o-3p | MIMAT0005919 | 115.90 | 131.30 | 108.92 | 119.18 |
| hsa-miR-548p | MIMAT0005934 | 53.83 | 46.66 | 111.03 | 92.61 |
| hsa-miR-548q | MIMAT0011163 | 107.40 | 119.13 | 116.49 | 121.05 |
| hsa-miR-548s | MIMAT0014987 | 88.97 | 92.06 | 92.33 | 103.68 |
| hsa-miR-548t-5p | MIMAT0015009 | 129.26 | 134.97 | 74.13 | 100.27 |
| hsa-miR-548u | MIMAT0015013 | 37.60 | 43.57 | 92.51 | 111.74 |
| hsa-miR-548v | MIMAT0015020 | 114.94 | 119.66 | 105.87 | 112.02 |
| hsa-miR-548w | MIMAT0015060 | 126.02 | 110.08 | 104.02 | 116.45 |
| hsa-miR-548x | MIMAT0015081 | 91.77 | 90.27 | 85.77 | 104.51 |
| hsa-miR-548y | MIMAT0018354 | 126.17 | 116.38 | 136.58 | 121.78 |
| hsa-miR-548z | MIMAT0018446 | 88.46 | 93.25 | 109.52 | 99.01 |
| hsa-miR-549a | MIMAT0003333 | 93.20 | 119.07 | 75.75 | 82.51 |
| hsa-miR-550a-3p | MIMAT0003257 | 85.49 | 106.31 | 115.03 | 104.09 |
| hsa-miR-550a-5p | MIMAT0004800 | 93.78 | 106.37 | 81.72 | 85.83 |
| hsa-miR-550b-3p | MIMAT0018445 | 116.06 | 77.66 | 98.69 | 71.76 |
| hsa-miR-551a | MIMAT0003214 | 134.06 | 119.13 | 126.79 | 127.27 |
| hsa-miR-551b-3p | MIMAT0003233 | 120.80 | 124.02 | 125.47 | 120.36 |
| hsa-miR-551b-5p | MIMAT0004794 | 110.44 | 119.87 | 89.52 | 80.18 |
| hsa-miR-552 | MIMAT0003215 | 54.23 | 63.07 | 68.29 | 80.92 |
| hsa-miR-553 | MIMAT0003216 | 145.63 | 121.75 | 117.24 | 96.63 |
| hsa-miR-554 | MIMAT0003217 | 71.42 | 99.95 | 88.43 | 97.82 |
| hsa-miR-555 | MIMAT0003219 | 98.26 | 117.65 | 114.53 | 119.88 |
| hsa-miR-556-3p | MIMAT0004793 | 166.97 | 214.59 | 139.64 | 151.56 |
| hsa-miR-556-5p | MIMAT0003220 | 71.76 | 83.73 | 105.94 | 112.97 |
| hsa-miR-557 | MIMAT0003221 | 81.77 | 80.92 | 107.82 | 92.22 |
| hsa-miR-558 | MIMAT0003222 | 94.20 | 83.14 | 94.27 | 91.42 |
| hsa-miR-559 | MIMAT0003223 | 105.62 | 90.38 | 132.43 | 114.57 |
| hsa-miR-561-3p | MIMAT0003225 | 122.98 | 103.15 | 117.76 | 116.60 |
| hsa-miR-562 | MIMAT0003226 | 102.21 | 78.40 | 115.49 | 129.94 |
| hsa-miR-563 | MIMAT0003227 | 106.18 | 95.35 | 143.68 | 126.54 |
| hsa-miR-564 | MIMAT0003228 | 100.47 | 118.21 | 103.73 | 101.09 |
| hsa-miR-566 | MIMAT0003230 | 113.13 | 108.91 | 104.51 | 92.30 |
| hsa-miR-567 | MIMAT0003231 | 117.48 | 89.11 | 121.89 | 106.66 |
| hsa-miR-568 | MIMAT0003232 | 94.24 | 94.35 | 99.47 | 114.49 |
| hsa-miR-569 | MIMAT0003234 | 81.25 | 81.01 | 67.65 | 99.76 |
| hsa-miR-570-3p | MIMAT0003235 | 49.76 | 66.94 | 71.24 | 77.34 |
| hsa-miR-571 | MIMAT0003236 | 107.67 | 78.40 | 118.41 | 120.47 |
| hsa-miR-572 | MIMAT0003237 | 88.36 | 77.57 | 103.25 | 103.53 |
| hsa-miR-573 | MIMAT0003238 | 136.29 | 125.80 | 125.60 | 133.26 |
| hsa-miR-574-3p | MIMAT0003239 | 113.10 | 125.65 | 94.22 | 100.65 |
| hsa-miR-574-5p | MIMAT0004795 | 103.66 | 110.34 | 119.31 | 99.30 |
| hsa-miR-575 | MIMAT0003240 | 101.05 | 119.30 | 104.48 | 125.53 |
| hsa-miR-576-3p | MIMAT0004796 | 99.52 | 77.80 | 108.49 | 113.36 |
| hsa-miR-576-5p | MIMAT0003241 | 121.62 | 139.37 | 98.94 | 119.51 |
| hsa-miR-577 | MIMAT0003242 | 129.01 | 118.23 | 118.17 | 106.30 |
| hsa-miR-578 | MIMAT0003243 | 36.24 | 25.42 | 92.55 | 96.92 |
| hsa-miR-579 | MIMAT0003244 | 119.91 | 101.82 | 88.83 | 78.31 |
| hsa-miR-580 | MIMAT0003245 | 84.13 | 110.29 | 83.34 | 77.92 |
| hsa-miR-581 | MIMAT0003246 | 119.23 | 95.48 | 112.77 | 117.65 |
| hsa-miR-582-3p | MIMAT0004797 | 99.16 | 114.48 | 91.38 | 110.64 |
| hsa-miR-582-5p | MIMAT0003247 | 189.17 | 216.00 | 73.30 | 89.05 |
| hsa-miR-583 | MIMAT0003248 | 114.94 | 109.23 | 91.18 | 92.58 |
| hsa-miR-584-5p | MIMAT0003249 | 69.99 | 68.86 | 79.47 | 76.33 |
| hsa-miR-585 | MIMAT0003250 | 98.12 | 86.08 | 97.84 | 127.84 |
| hsa-miR-586 | MIMAT0003252 | 134.76 | 154.64 | 101.47 | 113.44 |
| hsa-miR-587 | MIMAT0003253 | 106.78 | 108.51 | 89.13 | 67.39 |
| hsa-miR-588 | MIMAT0003255 | 110.85 | 123.14 | 93.08 | 84.88 |
| hsa-miR-589-3p | MIMAT0003256 | 112.30 | 105.47 | 103.22 | 109.01 |
| hsa-miR-589-5p | MIMAT0004799 | 96.49 | 83.97 | 91.42 | 97.85 |
| hsa-miR-590-3p | MIMAT0004801 | 98.12 | 98.37 | 103.94 | 107.26 |
| hsa-miR-590-5p | MIMAT0003258 | 132.32 | 132.09 | 110.04 | 96.57 |
| hsa-miR-591 | MIMAT0003259 | 81.30 | 78.96 | 86.34 | 92.05 |
| hsa-miR-592 | MIMAT0003260 | 119.44 | 112.16 | 111.90 | 120.11 |
| hsa-miR-593-3p | MIMAT0004802 | 87.86 | 115.09 | 109.29 | 96.21 |
| hsa-miR-593-5p | MIMAT0003261 | 49.59 | 52.20 | 123.81 | 118.36 |
| hsa-miR-595 | MIMAT0003263 | 131.07 | 136.90 | 100.66 | 100.55 |
| hsa-miR-596 | MIMAT0003264 | 130.00 | 95.35 | 58.61 | 61.93 |
| hsa-miR-597 | MIMAT0003265 | 178.63 | 153.83 | 66.09 | 85.66 |
| hsa-miR-598 | MIMAT0003266 | 117.10 | 111.45 | 99.30 | 80.76 |
| hsa-miR-599 | MIMAT0003267 | 112.18 | 124.25 | 89.86 | 88.60 |
| hsa-miR-600 | MIMAT0003268 | 134.72 | 149.58 | 103.97 | 120.53 |
| hsa-miR-601 | MIMAT0003269 | 92.80 | 94.52 | 86.00 | 106.79 |
| hsa-miR-602 | MIMAT0003270 | 120.85 | 119.45 | 134.62 | 145.56 |
| hsa-miR-603 | MIMAT0003271 | 120.21 | 87.86 | 99.24 | 99.90 |
| hsa-miR-604 | MIMAT0003272 | 133.97 | 119.08 | 137.67 | 124.68 |
| hsa-miR-605 | MIMAT0003273 | 137.68 | 135.98 | 80.57 | 86.13 |
| hsa-miR-606 | MIMAT0003274 | 111.50 | 105.12 | 91.75 | 95.78 |
| hsa-miR-607 | MIMAT0003275 | 90.25 | 97.94 | 94.76 | 111.40 |
| hsa-miR-608 | MIMAT0003276 | 41.01 | 42.25 | 125.30 | 122.49 |
| hsa-miR-609 | MIMAT0003277 | 81.87 | 88.57 | 100.80 | 124.03 |
| hsa-miR-610 | MIMAT0003278 | 167.45 | 190.86 | 124.00 | 131.65 |
| hsa-miR-611 | MIMAT0003279 | 116.54 | 120.62 | 115.02 | 95.75 |
| hsa-miR-612 | MIMAT0003280 | 70.35 | 70.08 | 88.83 | 94.42 |
| hsa-miR-613 | MIMAT0003281 | 134.37 | 109.55 | 91.69 | 78.40 |
| hsa-miR-614 | MIMAT0003282 | 89.95 | 112.65 | 119.47 | 113.59 |
| hsa-miR-615-3p | MIMAT0003283 | 54.98 | 47.66 | 115.99 | 129.44 |
| hsa-miR-615-5p | MIMAT0004804 | 120.93 | 121.01 | 99.79 | 99.48 |
| hsa-miR-616-3p | MIMAT0004805 | 132.32 | 115.43 | 123.90 | 115.96 |
| hsa-miR-616-5p | MIMAT0003284 | 118.77 | 112.40 | 117.82 | 94.15 |
| hsa-miR-617 | MIMAT0003286 | 45.95 | 78.08 | 83.47 | 80.71 |
| hsa-miR-618 | MIMAT0003287 | 97.03 | 108.61 | 85.83 | 105.64 |
| hsa-miR-619 | MIMAT0003288 | 118.19 | 125.35 | 83.43 | 123.09 |
| hsa-miR-620 | MIMAT0003289 | 121.70 | 89.85 | 96.16 | 92.90 |
| hsa-miR-621 | MIMAT0003290 | 79.47 | 112.01 | 113.45 | 127.01 |
| hsa-miR-622 | MIMAT0003291 | 94.45 | 90.18 | 90.50 | 101.59 |
| hsa-miR-623 | MIMAT0003292 | 73.99 | 108.45 | 101.60 | 81.24 |
| hsa-miR-624-3p | MIMAT0004807 | 84.73 | 97.65 | 104.54 | 118.42 |
| hsa-miR-624-5p | MIMAT0003293 | 92.82 | 83.62 | 134.99 | 126.51 |
| hsa-miR-625-3p | MIMAT0004808 | 110.23 | 109.75 | 102.98 | 112.64 |
| hsa-miR-625-5p | MIMAT0003294 | 113.73 | 87.27 | 90.08 | 77.57 |
| hsa-miR-626 | MIMAT0003295 | 87.14 | 87.23 | 95.28 | 90.54 |
| hsa-miR-627 | MIMAT0003296 | 117.60 | 124.17 | 99.75 | 101.53 |
| hsa-miR-628-3p | MIMAT0003297 | 94.97 | 91.77 | 100.55 | 96.31 |
| hsa-miR-628-5p | MIMAT0004809 | 36.17 | 37.29 | 87.81 | 76.46 |
| hsa-miR-629-3p | MIMAT0003298 | 85.17 | 98.23 | 75.40 | 72.00 |
| hsa-miR-629-5p | MIMAT0004810 | 69.99 | 70.60 | 87.15 | 89.40 |
| hsa-miR-630 | MIMAT0003299 | 168.45 | 149.05 | 107.61 | 75.35 |
| hsa-miR-631 | MIMAT0003300 | 113.81 | 123.69 | 87.93 | 94.95 |
| hsa-miR-632 | MIMAT0003302 | 107.81 | 106.51 | 76.14 | 107.27 |
| hsa-miR-633 | MIMAT0003303 | 86.25 | 85.91 | 92.53 | 82.26 |
| hsa-miR-634 | MIMAT0003304 | 40.19 | 67.38 | 149.97 | 150.99 |
| hsa-miR-635 | MIMAT0003305 | 77.40 | 91.11 | 83.02 | 94.48 |
| hsa-miR-636 | MIMAT0003306 | 123.78 | 106.25 | 103.68 | 92.53 |
| hsa-miR-637 | MIMAT0003307 | 85.38 | 84.55 | 95.55 | 114.24 |
| hsa-miR-638 | MIMAT0003308 | 81.94 | 90.45 | 108.17 | 99.90 |
| hsa-miR-639 | MIMAT0003309 | 83.07 | 69.73 | 100.30 | 85.15 |
| hsa-miR-640 | MIMAT0003310 | 91.75 | 77.92 | 98.29 | 80.02 |
| hsa-miR-641 | MIMAT0003311 | 87.68 | 114.19 | 87.51 | 99.53 |
| hsa-miR-642a-5p | MIMAT0003312 | 101.22 | 92.81 | 121.46 | 126.07 |
| hsa-miR-642b-3p | MIMAT0018444 | 69.80 | 85.48 | 93.46 | 86.51 |
| hsa-miR-643 | MIMAT0003313 | 137.30 | 103.77 | 89.86 | 103.74 |
| hsa-miR-644a | MIMAT0003314 | 63.07 | 61.89 | 79.84 | 77.96 |
| hsa-miR-645 | MIMAT0003315 | 121.16 | 103.71 | 113.91 | 110.43 |
| hsa-miR-646 | MIMAT0003316 | 65.35 | 84.36 | 69.58 | 90.23 |
| hsa-miR-647 | MIMAT0003317 | 97.61 | 97.30 | 82.19 | 98.82 |
| hsa-miR-648 | MIMAT0003318 | 94.90 | 112.36 | 105.58 | 116.13 |
| hsa-miR-649 | MIMAT0003319 | 71.95 | 116.54 | 85.70 | 107.17 |
| hsa-miR-650 | MIMAT0003320 | 94.28 | 81.19 | 119.84 | 123.73 |
| hsa-miR-651 | MIMAT0003321 | 72.23 | 77.48 | 85.09 | 81.03 |
| hsa-miR-652-3p | MIMAT0003322 | 73.10 | 105.15 | 117.80 | 112.21 |
| hsa-miR-653 | MIMAT0003328 | 35.41 | 45.32 | 84.29 | 95.20 |
| hsa-miR-654-3p | MIMAT0004814 | 165.29 | 171.83 | 87.06 | 82.51 |
| hsa-miR-654-5p | MIMAT0003330 | 96.31 | 70.55 | 102.90 | 116.48 |
| hsa-miR-655 | MIMAT0003331 | 37.88 | 35.70 | 76.69 | 62.99 |
| hsa-miR-656 | MIMAT0003332 | 109.51 | 101.46 | 88.58 | 81.86 |
| hsa-miR-657 | MIMAT0003335 | 103.21 | 113.15 | 103.41 | 131.52 |
| hsa-miR-658 | MIMAT0003336 | 98.26 | 102.80 | 115.02 | 104.70 |
| hsa-miR-659-3p | MIMAT0003337 | 91.72 | 89.94 | 93.27 | 91.50 |
| hsa-miR-660-5p | MIMAT0003338 | 99.82 | 110.59 | 88.72 | 109.69 |
| hsa-miR-661 | MIMAT0003324 | 85.17 | 81.37 | 77.64 | 77.33 |
| hsa-miR-662 | MIMAT0003325 | 142.02 | 148.84 | 104.51 | 120.61 |
| hsa-miR-663a | MIMAT0003326 | 94.28 | 108.69 | 93.54 | 115.28 |
| hsa-miR-663b | MIMAT0005867 | 109.04 | 117.53 | 120.75 | 105.99 |
| hsa-miR-664a-3p | MIMAT0005949 | 126.57 | 128.25 | 137.13 | 116.48 |
| hsa-miR-664a-5p | MIMAT0005948 | 94.37 | 92.67 | 90.59 | 92.90 |
| hsa-miR-665 | MIMAT0004952 | 78.74 | 78.19 | 97.22 | 108.24 |
| hsa-miR-668 | MIMAT0003881 | 165.38 | 150.52 | 102.71 | 103.56 |
| hsa-miR-670 | MIMAT0010357 | 68.55 | 62.72 | 76.27 | 93.80 |
| hsa-miR-671-3p | MIMAT0004819 | 105.48 | 90.56 | 93.37 | 70.21 |
| hsa-miR-671-5p | MIMAT0003880 | 109.95 | 112.56 | 103.46 | 84.10 |
| hsa-miR-675-3p | MIMAT0006790 | 88.75 | 121.94 | 77.66 | 69.22 |
| hsa-miR-675-5p | MIMAT0004284 | 90.28 | 89.87 | 94.29 | 98.64 |
| hsa-miR-676-3p | MIMAT0018204 | 101.25 | 92.18 | 118.13 | 121.46 |
| hsa-miR-676-5p | MIMAT0018203 | 112.15 | 134.47 | 74.40 | 74.27 |
| hsa-miR-708-3p | MIMAT0004927 | 93.41 | 103.93 | 114.80 | 98.90 |
| hsa-miR-708-5p | MIMAT0004926 | 61.24 | 74.31 | 49.38 | 50.05 |
| hsa-miR-711 | MIMAT0012734 | 83.84 | 97.62 | 122.59 | 105.74 |
| hsa-miR-7-1-3p | MIMAT0004553 | 94.57 | 117.88 | 99.91 | 111.71 |
| hsa-miR-718 | MIMAT0012735 | 63.19 | 74.97 | 86.20 | 102.11 |
| hsa-miR-720 | MIMAT0005954 | 141.27 | 151.30 | 92.18 | 113.06 |
| hsa-miR-7-2-3p | MIMAT0004554 | 54.23 | 63.78 | 86.13 | 58.65 |
| hsa-miR-744-3p | MIMAT0004946 | 57.82 | 61.09 | 84.11 | 75.93 |
| hsa-miR-744-5p | MIMAT0004945 | 107.81 | 122.87 | 105.94 | 90.05 |
| hsa-miR-758-3p | MIMAT0003879 | 105.83 | 99.86 | 80.04 | 80.50 |
| hsa-miR-759 | MIMAT0010497 | 82.42 | 86.01 | 81.71 | 81.63 |
| hsa-miR-7-5p | MIMAT0000252 | 110.83 | 135.51 | 134.86 | 155.41 |
| hsa-miR-760 | MIMAT0004957 | 93.26 | 88.51 | 94.77 | 90.46 |
| hsa-miR-761 | MIMAT0010364 | 94.41 | 114.64 | 106.16 | 95.78 |
| hsa-miR-762 | MIMAT0010313 | 83.84 | 88.91 | 109.07 | 101.17 |
| hsa-miR-764 | MIMAT0010367 | 39.51 | 32.29 | 89.52 | 98.47 |
| hsa-miR-765 | MIMAT0003945 | 96.26 | 110.60 | 70.10 | 86.99 |
| hsa-miR-766-3p | MIMAT0003888 | 80.97 | 81.03 | 57.97 | 61.65 |
| hsa-miR-767-3p | MIMAT0003883 | 101.25 | 103.97 | 97.73 | 107.04 |
| hsa-miR-767-5p | MIMAT0003882 | 68.52 | 44.52 | 101.30 | 94.95 |
| hsa-miR-768-3p | MIMAT0003947 | 71.40 | 69.62 | 106.92 | 122.69 |
| hsa-miR-768-5p | MIMAT0003946 | 98.34 | 87.37 | 102.16 | 91.09 |
| hsa-miR-769-3p | MIMAT0003887 | 105.62 | 91.73 | 97.34 | 95.00 |
| hsa-miR-769-5p | MIMAT0003886 | 75.82 | 55.14 | 106.71 | 96.57 |
| hsa-miR-770-5p | MIMAT0003948 | 87.59 | 97.61 | 99.33 | 100.81 |
| hsa-miR-801 | MIMAT0004209 | 88.20 | 95.78 | 94.28 | 81.16 |
| hsa-miR-802 | MIMAT0004185 | 94.61 | 76.71 | 89.70 | 90.71 |
| hsa-miR-873-5p | MIMAT0004953 | 80.07 | 95.50 | 70.08 | 81.67 |
| hsa-miR-874 | MIMAT0004911 | 137.68 | 91.52 | 79.84 | 102.48 |
| hsa-miR-875-3p | MIMAT0004923 | 122.38 | 125.22 | 115.55 | 95.51 |
| hsa-miR-875-5p | MIMAT0004922 | 37.73 | 43.79 | 101.95 | 83.14 |
| hsa-miR-876-3p | MIMAT0004925 | 73.85 | 78.13 | 87.32 | 116.72 |
| hsa-miR-876-5p | MIMAT0004924 | 97.12 | 113.45 | 60.51 | 65.37 |
| hsa-miR-877-3p | MIMAT0004950 | 117.51 | 100.18 | 102.96 | 99.90 |
| hsa-miR-877-5p | MIMAT0004949 | 73.02 | 73.64 | 108.42 | 67.22 |
| hsa-miR-885-3p | MIMAT0004948 | 57.89 | 61.98 | 86.57 | 84.46 |
| hsa-miR-885-5p | MIMAT0004947 | 106.79 | 86.32 | 89.21 | 109.80 |
| hsa-miR-886-3p | MIMAT0004906 | 100.35 | 81.40 | 109.54 | 122.87 |
| hsa-miR-887 | MIMAT0004951 | 62.30 | 76.71 | 106.15 | 98.88 |
| hsa-miR-888-3p | MIMAT0004917 | 108.17 | 115.69 | 92.25 | 78.37 |
| hsa-miR-888-5p | MIMAT0004916 | 102.82 | 112.93 | 92.87 | 101.02 |
| hsa-miR-889 | MIMAT0004921 | 72.63 | 67.97 | 93.21 | 87.02 |
| hsa-miR-890 | MIMAT0004912 | 65.22 | 88.07 | 81.41 | 98.38 |
| hsa-miR-891a | MIMAT0004902 | 92.67 | 101.13 | 89.47 | 94.62 |
| hsa-miR-891b | MIMAT0004913 | 50.33 | 53.01 | 62.65 | 55.22 |
| hsa-miR-892a | MIMAT0004907 | 104.75 | 95.86 | 82.90 | 82.43 |
| hsa-miR-892b | MIMAT0004918 | 83.79 | 71.63 | 110.61 | 114.82 |
| hsa-miR-920 | MIMAT0004970 | 93.07 | 123.77 | 88.24 | 77.31 |
| hsa-miR-921 | MIMAT0004971 | 91.53 | 101.11 | 101.03 | 115.22 |
| hsa-miR-922 | MIMAT0004972 | 93.52 | 97.60 | 129.61 | 105.75 |
| hsa-miR-923 | MIMAT0004973 | 118.77 | 89.12 | 128.60 | 112.90 |
| hsa-miR-924 | MIMAT0004974 | 93.20 | 96.02 | 92.84 | 106.62 |
| hsa-miR-92a-1-5p | MIMAT0004507 | 108.45 | 114.19 | 97.01 | 109.34 |
| hsa-miR-92a-2-5p | MIMAT0004508 | 67.38 | 85.27 | 66.47 | 75.69 |
| hsa-miR-92a-3p | MIMAT0000092 | 48.52 | 38.78 | 93.53 | 93.68 |
| hsa-miR-92b-3p | MIMAT0003218 | 57.25 | 59.50 | 103.66 | 84.91 |
| hsa-miR-92b-5p | MIMAT0004792 | 111.46 | 105.36 | 99.51 | 116.88 |
| hsa-miR-933 | MIMAT0004976 | 65.24 | 77.59 | 101.95 | 123.13 |
| hsa-miR-93-3p | MIMAT0004509 | 124.65 | 82.27 | 142.11 | 122.47 |
| hsa-miR-934 | MIMAT0004977 | 132.53 | 142.14 | 108.65 | 109.76 |
| hsa-miR-935 | MIMAT0004978 | 187.10 | 169.72 | 144.41 | 140.35 |
| hsa-miR-93-5p | MIMAT0000093 | 86.70 | 83.41 | 116.88 | 130.70 |
| hsa-miR-936 | MIMAT0004979 | 116.68 | 118.78 | 103.33 | 111.71 |
| hsa-miR-937-3p | MIMAT0004980 | 119.13 | 105.37 | 112.64 | 111.38 |
| hsa-miR-938 | MIMAT0004981 | 123.02 | 111.39 | 121.89 | 126.93 |
| hsa-miR-939-5p | MIMAT0004982 | 110.59 | 108.45 | 112.27 | 130.58 |
| hsa-miR-9-3p | MIMAT0000442 | 107.51 | 105.23 | 100.13 | 105.42 |
| hsa-miR-940 | MIMAT0004983 | 77.40 | 57.21 | 96.75 | 119.53 |
| hsa-miR-941 | MIMAT0004984 | 136.56 | 146.05 | 119.31 | 108.48 |
| hsa-miR-942 | MIMAT0004985 | 111.97 | 110.97 | 90.50 | 78.62 |
| hsa-miR-943 | MIMAT0004986 | 78.15 | 118.04 | 92.26 | 112.17 |
| hsa-miR-944 | MIMAT0004987 | 128.29 | 122.80 | 110.05 | 94.11 |
| hsa-miR-95 | MIMAT0000094 | 136.14 | 127.26 | 109.80 | 86.46 |
| hsa-miR-9-5p | MIMAT0000441 | 55.86 | 71.68 | 75.75 | 83.11 |
| hsa-miR-96-3p | MIMAT0004510 | 100.66 | 77.09 | 116.55 | 104.68 |
| hsa-miR-96-5p | MIMAT0000095 | 114.35 | 112.02 | 120.88 | 109.32 |
| hsa-miR-98-5p | MIMAT0000096 | 22.99 | 38.54 | 68.25 | 57.76 |
| hsa-miR-99a-3p | MIMAT0004511 | 122.63 | 101.32 | 88.53 | 92.63 |
| hsa-miR-99a-5p | MIMAT0000097 | 102.71 | 82.64 | 104.46 | 91.93 |
| hsa-miR-99b-3p | MIMAT0004678 | 140.73 | 160.55 | 117.33 | 98.37 |
| hsa-miR-99b-5p | MIMAT0000689 | 116.91 | 134.23 | 93.36 | 101.82 |

SUPPLEMENTARY TABLE 2
Physical parameters in different mice transduced
with different shRNAs measured using dual-energy
x-ray absorptiometry (DEXA). Mice were transduced
with different shRNAs as described in FIG. 4. DEXA
was performed 3 months after the injection of viruses.
All KD mice showed significantly less fat weight.
Data are average + SD, Student t-test compared to controls.

| DEXA | shCtrl | shCasz1 | shZfp961 | sh(C + Z) |
|---|---|---|---|---|
| parameters | (n = 4) | (n = 4) | (n = 4) | (n = 5) |
| Total Weight | 23.11 ± 0.55 | 24.60 ± 1.32 | 23.59 ± 0.18 | 21.31 ± 1.50, |
| (gms) | | P = 1.72 | P = 0.86 | P = 0.06 |
| Soft Weight | 21.91 ± 0.49 | 23.61 ± 1.42 | 22.63 ± 0.13 | 20.34 ± 1.60 |
| (gms) | | P = 0.13 | P = 0.70 | P = 0.14 |
| Lean Weight | 12.82 ± 2.92 | 16.20 ± 2.92 | 16.38 ± 0.95 | 13.99 ± 2.75 |
| (gms) | | P = 0.19 | P = 0.17 | P = 0.88 |
| Fat Weight | 10.59 ± 0.75 | 7.41 ± 1.69 | 6.25 ± 0.86 | 6.35 ±1.75 |
| (gms) | | P = 0.001 | P = 0.0002 | p < 0.0001 |
| Fat | 46.46 ± 4.05 | 31.72 ± 8.83 | 27.64 ± 3.92 | 31.56 ± 0.94 |
| (% of body | | P = 0.003 | P = 0.0005 | P = 0.0003 |
| weight) | | | | |
| Bone | 1.25 ± 0.17 | 0.98 ± 0.11 | 0.96 ± 0.11 | 0.97 ± 0.18 |
| Mineral | | P = 0.08 | P = 0.052 | P = 0.05 |
| Content | | | | |
| (gms) | | | | |
| Bone | 88.66 ± 3.82 | 87.76 ± 3.72 | 90.52 ± 7.65 | 88.42 ± 6.85 |
| Mineral | | P = 0.99 | P = 0.94 | P = 0.99 |
| Density | | | | |
| (mg/cm²) | | | | |

SUPPLEMENTARY TABLE 3
Total weight gain was less in different mice transduced
with shRNAs to KD Casz1 and Zfp961. Mice were transduced
with different shRNAs as described in FIG. 5. DEXA was
performed 3 months after the injection of viruses. All
KD mice showed significantly less total; soft, lean and fat weights.
Data are average + SD, Student t-test compared to controls.

| | mPcsk9 + | mPcsk9 + | mPcsk9 + | mPcsk9 + |
|---|---|---|---|---|
| DEXA | shCtrl | shCasz1 | shZfp961 | sh(C + Z) |
| Parameters | (n = 4) | (n = 4) | (n = 4) | (n = 5) |
| Total Weight | 40.97 ± 0.82 | 34.18 ± 2.58 | 36.17 ± 1.47 | 30.58 ± 3.16 |
| (gms) | | P = 0.024 | P = 0.001 | P = 0.0004 |
| Soft Weight | 40.00 ± 0.81 | 33.28 ± 2.59 | 35.21 ± 1.38 | 29.61 ± 3.17 |
| (gms) | | P = 0.002 | P = 0.001 | P = 0.002 |
| Lean Weight | 27.05 ± 0.49 | 22.83 ± 1.35 | 23.47 ± 2.61 | 20.07 ± 2.54 |
| (gms) | | P = 0.001 | P = 0.041 | P = 0.0004 |
| Fat Weight | 13.17 ± 0.58 | 10.45 ± 1.37 | 10.24 ± 1.09 | 9.54 ± 1.01 |
| (gms) | | P = 0.013 | P = 0.005 | P = 0.0006 |
| Fat | 32.93 ± 1.12 | 31.31 ± 1.92 | 33.25 ± 8.01 | 32.27 ± 2.46 |
| (% of body | | P = 0.19 | P = 0.94 | P = 0.64 |
| weight) | | | | |
| Bone Mineral | 0.98 ± 0.01 | 0.90 ± 0.02 | 0.96 ± 0.10 | 0.96 ± 0.08 |
| Content (gms) | | P = 0.003 | P = 0.82 | P = 0.76 |
| Bone Mineral | 86.32 ± 1.50 | 85.38 ± 2.26 | 86.12 ± 0.77 | 89.55 ± 2.30 |
| Density | | P = 0.51 | P = 0.81 | P = 0.04 |
| (mg/cm²) | | | | |

Data are average + SD. Student t-test.

What is claimed is:

1. A method comprising administering to an individual in need thereof a composition comprising:
i) miRNA-541-3P or a modified version thereof; or
ii) an agent other than the miRNA-541-3P or the modified version thereof that inhibits the function or expression of ZNF101; or
iii) an agent other than the miRNA-541-3P or the modified version thereof that inhibits the function or expression of CASZ1;
iv) or a combination comprising at least two of i), ii) and iii).

2. The method of claim 1, comprising administering the miRNA-541-3P.

3. The method of claim 1, comprising administering the agent of i), and wherein the modified version of the miRNA-541-3P comprises a modified ribosugar modification, a modified ribonucleotide, a modified inter-nucleoside linkage, or a combination thereof.

4. The method of claim 1, comprising administering the agent of ii).

5. The method of claim 4, wherein the agent comprises an RNAi agent that inhibits expression of the ZNF101.

6. The method of claim 4, wherein the agent comprises a CRISPR system that modifies a genomic ZNF101 coding region such that expression of ZNF101 protein is reduced.

7. The method of claim 4, comprising administering the agent of iii) wherein the agent comprises an RNAi agent that inhibits expression of the CASZ1.

8. The method of claim 4, comprising administering the agent of iii) wherein the agent comprises a CRISPR system that modifies a CASZ1 genomic coding region such expression of CASZ1 protein is reduced.

9. The method of claim 4, wherein the miRNA-541-3P or the modified version thereof or the agent is administered to liver cells within the individual.

10. The method of claim 1, wherein the individual is in need of treatment or prophylaxis of atherosclerosis, or is in need of a reduction of plasma low-density lipoprotein (LDL), or is in need of an increase of plasma high-density lipoprotein (HDL), or a combination thereof.

11. The method of claim 1, wherein both the reduction of the plasma LDL and the increase of the HDL occur.

12. The method of claim 1, wherein the individual is in need of treatment for the atherosclerosis.

13. The method of claim 9, wherein the individual is in need of treatment or prophylaxis of atherosclerosis, or is in need of a reduction of plasma LDL, or is in need of an increase of plasma HDL, or a combination thereof.

14. The method of claim 9, wherein both the reduction of the plasma LDL and the increase of the plasma HDL occur.

15. The method of claim 9, wherein the individual is in need of treatment for the atherosclerosis.

16. The method of claim 1, wherein expression of apoB is decreased and expression of apoA1 is increased.

17. The method of claim 9, wherein expression of apoB is decreased and expression of apoA1 is increased.
